(19) United States
(12) Patent Application Publication
(10) Pub. No.: US 2006/0160178 A1
(43) Pub. Date: Jul. 20, 2006

(54) Methods of generating antibody diversity in vitro

(76) Inventors: Jonathan Rothberg, Guilford, CT (US); Michael Weiner, Guilford, CT (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/991,309

(22) Filed: Nov. 17, 2004

Related U.S. Application Data
(60) Provisional application No. 60/520,881, filed on Nov. 17, 2003.
(60) Provisional application No. 60/581,072, filed on Jun. 17, 2004.

(51) Int. Cl. C12P 21/06; C07K 16/18; C12N 1/21; C07H 21/04; C12N 5/06; C12N 1/18; C12N 15/74

(52) U.S. Cl. 435/69.1; 435/320.1; 435/326; 435/252.3; 435/254.2; 530/388.1; 536/23.53

(57) ABSTRACT

The present invention provides a high throughput method for generating fully human monoclonal antibodies.

### RELATED APPLICATIONS

This application claims priority to U.S. Ser. No. 60/520,881 filed Nov. 17, 2003 and U.S. Ser. No. 60,581,072 filed Jun. 17, 2004, the contents of which are incorporated herein by reference in their entireties.

### FIELD OF THE INVENTION

The invention relates to generally to production of human monoclonal antibodies.

### BACKGROUND OF THE INVENTION

Structurally, the simplest antibody comprises four polypeptide chains, two heavy (H) chains and two light (L) chains inter-connected by disulphide bonds. The light chains exist in two distinct forms called kappa (κ) and lambda (λ). Each chain has a constant region (C) and a variable region (V). Each chain is organized into a series of domains. The light chains have two domains, corresponding to the C region and the other to the V region. The heavy chains have four domains, one corresponding to the V region and three domains (1, 2 and 3) in the C region. The antibody has two arms (each arm being a Fab region), each of which has a V_{L} and a V_{H} region associated with each other. It is this pair of V regions (V_{L} and V_{H}) that differ from one antibody to another, and which together are responsible for recognizing the antigen and providing an antigen binding site. Each V region is made up from three CDRs separated by four framework regions. The CDRs are the most variable part of the variable regions, and they perform the critical antigen binding function. The CDR regions are derived from many potential germ line sequences via a complex process involving recombination, mutation and selection. The function of binding antigens can be performed by fragments of a whole antibody. An example of a binding fragment is the Fv fragment consisting of the V_{L} and V_{H} domains of a single arm of an antibody.

### SUMMARY OF THE INVENTION

The invention allows the production and identification an antibody or fragement thereof specific for any antigen in an extremely high throughput and cost effective manner. Preferably, the antibody is a single chain antibody (scFV). The antibody is fully human.

A nucleic acid encoding an antibody is produced by providing a plurality of oligonucleotides including oligonucleotides encoding the framework regions and the complementarity determining regions (CDRs) of an antibody. The nucleic acid is single stranded. Alternatively, the nucleic acid is double stranded. The olignucleotides acid are constructed from any source of nucleic acid, e.g., any cell, tissue, or organism, database of known sequences and can be generated by any art-recognized method. The CDRs or hypervariable regions are regions that show the most variability within the heavy and light chains. The framework regions are the regions between CDRs. The amino acids and nucleotides defining these regions are know to those skilled in the art.

The oligonucleotides are annealed to yield a plurality of primed oligonucleotide complexes and then amplified by combining the primed oligonucleotide complexes with a polymerase, one or more nucleotides, and optionally a ligase to yield a nucleic acid sequence encoding a single-chain. Amplification reactions are carried out using manual or automatic methods by methods known in the art such as the polymerase chain reaction. Suitable polymerases for amplification include, e.g., the DNA polymerases from Bacillus stearothermophilus, Thermus aquaticus, Pyrococcus furiosis, Thermococcus litoralis, and Thermus thermophilus, bacteriophage T4 and T7, and the E. coli DNA polymerase I Klenow fragment. Suitable RNA-directed DNA polymerases include, e.g., the reverse transcriptase from the Avian Myeloblastosis Virus, the reverse transcriptase from the Moloney Murine Leukemia Virus, and the reverse transcriptase from the Human Immunodeficiency Virus-I.

The reaction products are isolated and analyzed using any of several methods that are well-known in the art such size exclusion. Preferably, gel electrophoresis is used to rapidly resolve and identify each of the amplified sequences. Alternatively, the amplification reaction mixture may be treated with one or more enzymes prior to electrophoresis, for example, restriction endonucleases. Alternative methods of product analysis include without limitation dot-blot hybridization with allele-specific oligonucleotides, SSCP, sequencing, i.e., by hybridization or incorporation of fluorophores or dideoxynucleotides, or by extension reactions.

The plurality of oligonucleotides include at least one oligonucleotide encoding a 5′ Framework I sequence, a first germline-encoding CDR1 amino acid sequence, and a 3′ Framework 2 sequence; at least one oligonucleotide encoding a 5′ Framework 2 sequence, a first germline-encoding CDR2 amino acid sequence, and a 3′ Framework 3 sequence, where a region of the 3′ Framework 2 sequence is complementary to a region of the 5′ Framework 2 sequence; at least one oligonucleotide encoding a 5′ Framework 3 sequence, a first germline-encoding CDR3 amino acid sequence, and a 3′ Framework 4 sequence, where a region of the 3′ Framework 3 sequence is complementary to a region of the 5′ Framework 3 sequence; at least one oligonucleotide encoding a 5′ Framework 4 sequence, a linker sequence, and a 3′ Framework 5 sequence, where a region of the 3′ Framework 4 sequence is complementary to a region of the 5′ Framework 4 sequence; at least one oligonucleotide encoding a 5′ Framework 5 sequence, a first germline-encoding CDR4 amino acid sequence, and a 3′ Framework 6 sequence, where a region of the 3′ Framework 5 sequence is complementary to a region of the 5′ Framework 5 sequence; at least one oligonucleotide encoding a 5′ Framework 6 sequence, a first germline-encoding CDR5 amino acid sequence, and a 3′ Framework 7 sequence, where a region of the 3′ Framework 6 sequence is complementary to a region of the 5′ Framework 6 sequence; and at least one oligonucleotide encoding a 5′ Framework 7 sequence, a first germline-encoding CDR6 amino acid sequence, and a 3′ Framework 8 sequence, where a region of the 3′ Framework 8 sequence is complementary to a region of the 5′ Framework 7 sequence. In some aspects, the plurality further contains olignucleotide complementary to the aforementioned olignucleotides.

Optionally, plurality contains oligonucleotides having a second, third, forth, fifth, tenth, twentieth, fiftieth or a hundredth CDR amino acid sequence. Each CDR encodes for a different amino acid sequence. For example the plurality contains an oligonucleotide encoding a 5′ Framework 1 sequence, a second germline-encoding CDR1 amino acid sequence, and a 3′ Framework 2 sequence, where said second germline-encoding CDR1 amino acid sequence is different than the first germline-encoding CDR1 amino acid sequence.

By CDR1, CDR2, CDR3, CDR4, CDR5, and CDR6 is meant to represent one of each of the six CDR regions contained in a native antibody. For example, CDR1, CDR2 and CDR3 correspond to the three light chain CDRs, whereas, CDR4, CDR5, and CDR6 correspond to the three heavy chain CDRs or visa versa. Likewise, Framework 1, Framework 2, Framework 3, Framework 4, Framework 5, Framework 6, Framework 7, and Framework 8 is meant to represent one of each of regions interspersed between the CDRs in a native antibody. Preferably, the framework regions are derived from the same chain as the CDRs. For example if CDR1, CDR2 and CDR3 are light chain CDRs then Framework 1, Framework 2, Framework 3, Framework 4 are light chain frameworks.

Optionally, the plurality contains at least one oligonucleotide encoding a 5′ Framework 1 sequence, a germline-encoding CDR1 amino acid sequence, and a 3′ Framework 2 sequence comprises 5′ to the 5′ Framework I sequence a first priming sequence and/or at least one oligonucleotide encoding a 5′ Framework 8 sequence, a germline-encoding CDR6 amino acid sequence, and a 3′ Framework 8 sequence comprises 3′ to the 3′ Framework 8 sequence a second priming sequence. The first and second priming sequence are sequences that contain the furthermost 5′ and 3′ sequence of the nucleic acid construct, allowing the nucleic acid sequence encoding the antibody to be amplified.

In various aspects of the methods of the invention include expressing the protein encoded by the nucleic acid and screening the protein for a desired property, e.g., specific bind to an antigen. Screening is done by methods known in the art such as a yeast two-hybrid system, phage display, biopanning or an ELISA assay. The nucleic acids encoding the protein are partitioned. By partitioned is meant that the nucleic acid sequence of nucleotide encoding the protein of interest is identified or “decoded”. The partitioned nucleic acids are amplified to produce an enriched mixture of the nucleic acid encoding the proteins of the desired property. By amplifying is meant the nucleic acid sequences utilized to encode the scFV are reshuffled to produce an “affinity matured” antibody. An enriched mixture encodes for a protein that binds an antigen with high affinity. High affinity is at a Kd of at least 10⁻⁵. For example the Kd is 10⁻⁶, 10⁻⁷, 10⁻⁸, 10⁻⁹, 10⁻¹⁰, 10⁻¹¹, or 10⁻¹².

Also included in the invention are the nucleic acid encoding a single-chain antibody vector containing the nucleic acid, a host cell, e.g. bacterial cell, yeast cell or mammalian cell contain the vector, a nucleic acid single chain antibody library, kits containing the nucleic acids and the single chain antibody polypeptide obtained by the methods of the invention.

The present invention further provides a multi-functional vector, e.g., a plasmid vector, a BAC vector, a cosmid vector, or a YAC vector, useful in a yeast two-hybrid (Y2H) system and in a phage display system, and is, optionally, useful for polypeptide expression in E. coli. The expression vector of the invention contains a polynucleotide sequence that includes an activation domain, e.g. a B42 activation domain, a VP16 activation domain, a GAL4 activation domain, or an NF-κB activation domain, a yeast promoter sequence, e.g., GAL4, a polynucleotide sequence encoding a bacteriophage coat protein or fragment thereof, e.g. gpIII, gpVII, and gpVII, a bacterial promoter sequence, e.g., lacl, lacZ, T3, T7, gpt, lambda P_{R}, P_{L} and TRP, a protein fusion site, and a suppressible stop codon such as the amber stop codon. Optionally, the expression vector includes one or more of the following polynucleotide sequences: (i) a nuclear localization sequence; (ii) an epitope tag; (iii) a yeast transcription termination signal; (iv) an origin of replication; (v) a selectable marker, e.g. a yeast selectable marker of a bacterial selectable marker; and (vi) an additional promoter. a polynucleotide sequence encoding a bacteriophage coat protein or fragment thereof in functional combination with a bacterial promoter sequence. The vector is capable of expressing a polypeptide sequence in two or more species, e.g., a yeast, a bacterium, or a mammal. The components of the vector are in functional combination. For example, the yeast activation domain is located downstream of the yeast promoter sequence, the bacterial promoter sequence is in-frame with and downstream of the activation domain, the protein fusion site is downstream of the bacterial promoter sequence, the suppressible stop codon is downstream of the protein fusion site, and the polynucleotide sequence encoding the bacteriophage coat protein is downstream of the suppressible stop codon.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic representation of antibody gene synthesis.

FIG. 2 is a schematic representation of non-palindrome recognition sequence gene shuffling.

FIG. 3 is a schematic representation of the automated yeast two-hybrid screen for random DNAs

FIG. 4 is a schematic representation of identification of CDR sequence of the randomly generated synthetic antibody by “zip coding”.

FIG. 5 is a schematic representation of identification of CDR sequence of the randomly generated synthetic antibody by “zip coding”.

FIG. 6 is a schematic representation of the automated yeast two-hybrid screen of random cDNA libraries.

FIG. 7A is a schematic illustration of CDR shuffling. FIG. 7B is a photographic representation of an ELISA on scFv D2E7 fused to M13 SAM3.

FIG. 8A demonstrates the identification of CDRs in antibody genes.

FIG. 8B is a schematic illustration showing that the binding of CDRs occurs independently of each other, so the use of this schema creates a library of maximum diversity. The 10-100 CDRs for each of the six CDRs can be shuffled using non-palindrome restriction endonuclease enzymes to create a 10⁶-10¹² CDR library.

FIG. 8C is a schematic illustration demonstrating restriction enzyme cleavage downstream from the recognition site. CDRs are chosen with at least one internal amino-acid sequence which can recode a non-palindrome site, here, the FokI (GGATG 9/13) site.

FIG. 9 provides the nucleotide and amino acid sequences of a synthetic D2E7. The blue-highlighted sequences represent alternating sets of CDR oligonucleotide primers that overlap in the D2E7 gene.

FIG. 10 is a schematic illustration of the pCH103 cloning vector, that provides expression of target proteins in yeast and E. coli. As shown, the vector contains i) GAL1 promoter for expression of genes cloned into pYESTrp2, expression is constitutive in L40 and inducible in EGY48/pSH18-34, ii) V5 epitope, that allows detection of fusion protein(s) using the Anti-V5 antibody, iii) SV40 large T antigen nuclear localization sequence (NLS), which localizes fusions to the nucleus for potential interaction with LexA fusions, iv) B42 activation domain (AD) that allows expression of reporter genes when brought into proximity with the LexA DNA binding domain (DBD) by two interacting proteins, v) CYC1 transcription termination signal, which permits efficient termination and stabilization of mRNA, vi) TRP1 gene for auxotrophic selection of the plasmid in Trp-yeast hosts (e.g., L40 or EGY48/pSH18-34), vii) 2μ origin for maintenance and high-copy replication in yeast, viii) fl origin for rescue of single-strand DNA, ix) encoded E. coli lac promoter (P_{E.c.}) for controlled expression of gpIII fusion construct, x) M13 gpIII gene for phage display fusions to the gpIII protein of M13, xi) Protein fusion site, which functions as a site where proteins are cloned into the multiple cloning site of the pCH103 vector.

FIG. 11A. is a schematic illustration of the two hybrid system, wherein two physically distinct functional domains are necessary: a DNA binding domain (DBD) and an activation domain (AD). In the Two-Hybrid System a known gene, X, is cloned into the “bait” vector. In the system developed by Brent, it is expressed as a fusion with the bacterial-derived LexA DBD. A second gene, Y, or a library of cDNAs encoding potential interactors is cloned in frame with the B42 AD. The “bait” and “prey” fusion constructs are transformed into one of several available yeast strains. In these yeast strains, the LexA operator is engineered into the 5′ flanking region of reporter genes. The LexA DBD-“bait” fusion will bind specifically to this engineered site. If the X and Y proteins interact, the B42 AD is brought into proximity with the LexA DBD and transcription of the reporter genes is activated. Positive interactors are identified by plating on nutrient deficient medium and screening for β galactosidase activity. These positive clones can be analyzed by restriction analysis, PCR, or sequencing.

FIG. 11B is a schematic illustration of the pYESTrp2 vector (the ‘bait’ vector).

FIG. 12 discloses the nucleotide and amino acid sequences of the B42 activation domain and multiple cloning site.

FIG. 13A is a schematic illustration of an all-liquid format for high-throughput Y2H. The Y2H assay, which permits analysis of proteins in an intracellular setting, requires the expression of two fusion proteins. Individually, neither the DBD nor AD can activate transcription in yeast. (left) Creation of the arrayed cDNA library pooled subsets for the ‘Master Library Plates.’ For automated identification of interactors in the arrayed library, each pooled subset of the master library was aliquot to daughter plates. The mating mixture was then diluted and the outgrown yeast library was aliquot to separate 96-well plates, into which were seeded a bait-expressing strain of the opposite mating type. After mating, diploids were subjected to prototrophic selection and assayed for β galactosidase activity. FIG. 13B shows the results of Y2H analysis of the human nuclear receptor RXRα. Each well of the three 96-well microtiter plates represents β galactosidase assay on prototrophic-selected diploids arising from a pEG-RXRα bait plasmid-expressing yeast strain mated to ca. 1000 yeast clones of a pJD4.5 AD library. The right-most columns in each plate represent both positive and negative controls. CPRG was used as the chromogenic substrate. Dark-red wells are indicative of the presence of an interaction occurring within the well.

FIG. 14 is a schematic illustration showing the generation of the pCH103 vector.

FIG. 15 discloses the nucleotide and amino acid sequences of the region between the yeast B42 and M13gpIII genes, which encodes an open reading frame of the lac promoter. In yeast this sequence will be recognized as the first reading frame peptide sequence shown above. In E. coli, this region will be recognized as a lac promoter, with the start of translation indicated as fMET.

FIG. 16 is a schematic illustration showing the structure of the T7 phage particle. The capsid shell, head-tail connector, tail, and tail fibers are shown schematically. The diffraction pattern from polyheads (4) showing a hexamer capsid unit has been fit onto the surface of the icosahedral particle (diameter approx. 55 nm). The monomer units are in gray.

FIG. 17 is a schematic illustration of the bacterial two-hybrid system, in which a protein of interest (the bait) is fused to the full-length bacteriophage lambda cI repressor protein (lambda cI, 237 amino acids), which binds to the I operator. The target protein is fused to the N-terminal domain of the a-subunit of RNA polymerase (248 amino acids). When the bait and target interact, they recruit and stabilize the binding of RNA polymerase close to the promoter and activate the transcription of the His3 reporter gene. A second reporter gene, aadA, is expressed from the same promoter and confers resistance to streptomycin, providing an additional mechanism to validate the bait and target interaction.

FIG. 18A is a schematic illustration of the bacteriophage M13 particle, showing the gpIII protein (several thousand copies per phage particle) and to the gpIII protein (ca 5 copies per phage). FIG. 18B shows the phage display cycle. DNA encoding for millions of variants of certain ligands (e.g. peptides, proteins or fragments thereof) is batch-cloned into the phage genome as part of one of the phage coat proteins (pIII, pVI or pVIII). Large libraries containing millions of different ligands can be obtained by force cloning in E. coli. From these repertoires, phage carrying specific binding ligands can be isolated by a series of recursive cycles of selection on antigen, each of which involves binding, washing, elution and amplification.

FIG. 19A shows the cloning of the D2E7 gene into the pCH103 vector between the EcoRI and XhoI sites. FIG. 19B shows the analysis of the expressed D2E7 gene. 3+3 type phage expressing D2E7 were generated by infecting an overnight culture of XL-1 Blue E. coli carrying the pCH105 plasmid with M13K07 helper phage (NEB). Parallel cultures were incubated in the presence of varying concentrations of IPTG to increase D2E7-pIII fusion protein production. The resultant 3+3 phage show 11-17 fold increase in binding to TNFá (the D2E7 antigen) vs. BSA (as measured by A₄₀₅ in an ELISA). This TNFα specific signal is comparable to 33 type phage expressing D2E7.

FIG. 20 shows the cloning of the Jun gene (from pYESTrp-Jun) into the pCH103 vector between the EcoRI and XhoI sites. The fragment was sequence confirmed prior to restriction cloning. Testing pBNJ111.1 in Y2H. Jun/Fos have been shown to interact strongly during co-expression in S. cerevisiae (as measured by the ability to produce His prototrophy in a Y2H assay). A positive interaction has been observed during co-expression of pBNJ111.1 and pHybLex/Fos2. Colonies displaying His prototrophy during co-expression of pBNJ 111.1 and pHybLex/Fos2 are slower to manifest than those expressing pYESTrp-Jun and pHybLex/Fos2.

FIG. 21 is an illustration showing the synthesis of pCH103. Briefly the pCH103 vector was created by a XhoI restriction enzyme cleavage of pCH102 and an intra-molecular ligation. The final vector includes: (i) The Gall Yeast Promoter (ii) V5 epitope (iii) SV40 large T antigaen NLS sequence (iv) B42 activation domain (v) CYCl transcription termination signal (vi) TRP1 gene (vii) 2μ origin (viii) fl origin (ix) E. coli lac promoter (x) M13 gpII gene (xi) Xhol/EcoRI protein fusion site.

FIG. 22 is an illustration showing the DNA sequence of pCH103. Shown in bold is the truncated M13gpIII gene, italic is the lacP

FIG. 23 is an illustration showing the restriction digest map of pCH103. Enzymes without a site in pCH103; Acc65I (GˆGTAC_C) AflII (CATTAA_G) AleI (CACNN|NNGTG) ApaI (G_GGCCˆC) AscI (GGˆCGCG_CC) AsiSI (GCG_ATACGC) AvrII (CˆCTAG_G) BaeI (_NNNNNˆNNNNNNNNNNACNNNNGTAYCNNNNNNN_NNNNNˆ) BamHI (GˆGATC_C) BbvCI (CCˆTCA_GC) BclI (TˆGATC_A) BglII (AˆGATC_T) BlpI (GCˆTNA_GC) BmtI (G_CTAGAC) Bpu10I (CCˆTNA_GC) BsiWI (CˆGTAC_G) BsmI (GAATG_CNˆ) BspEI (TˆCCGG_A) BssHII (GˆCGCG_C) BstEIl (GˆGTNAC_C) BstXI (CCAN_NNNNˆNTGG) EcoNI (CCTNNˆN_NNAGG) FseI (GG_CCGGˆCC) KasI (GˆGCGC_C) KpnI (G_GTACˆC) MscI (TGG″CCA) NarI (GGˆCG_CC) NheI (GˆCTAG_C) NruI (TCG|CGA) PacI (TTA_ATˆTAA) PflFI (GACNˆN_NGTC) PflMI (CCAN_NNNˆNTGG) PmeI (GTTT|AAAC) PshAI (GACNN|NNGTC) PspOMI (GˆGGCC_C) RsrII (CGˆGWC_CG) SacI(G_AGCTˆC) SacII (CC_GCˆGG) SalI (GˆTCGA_C) SbfI (CC_TGCAˆGG) SexAI (AˆCCWGG_T) SfiI (GGCCN_NNNˆNGGCC) SfoI (GGC|GCC) SgrAI (CRˆCCGG_YG) SmaI (CCC|GGG) SphI (G_CATGˆC) Tth111I (GACNˆN_NGTC) XcmI (CCANNNN_NˆNNNNTGG) XmaI (CˆCCGG_G).

FIG. 24 is an illustration showing the B42/gpIII gene sequence of pCH103.

FIG. 25 is an illustration or the restriction digest map of MCS in pCH103.

FIG. 26 is an illustration of the multiple cloning site (i.e. protein fusion site) of pCH 103.

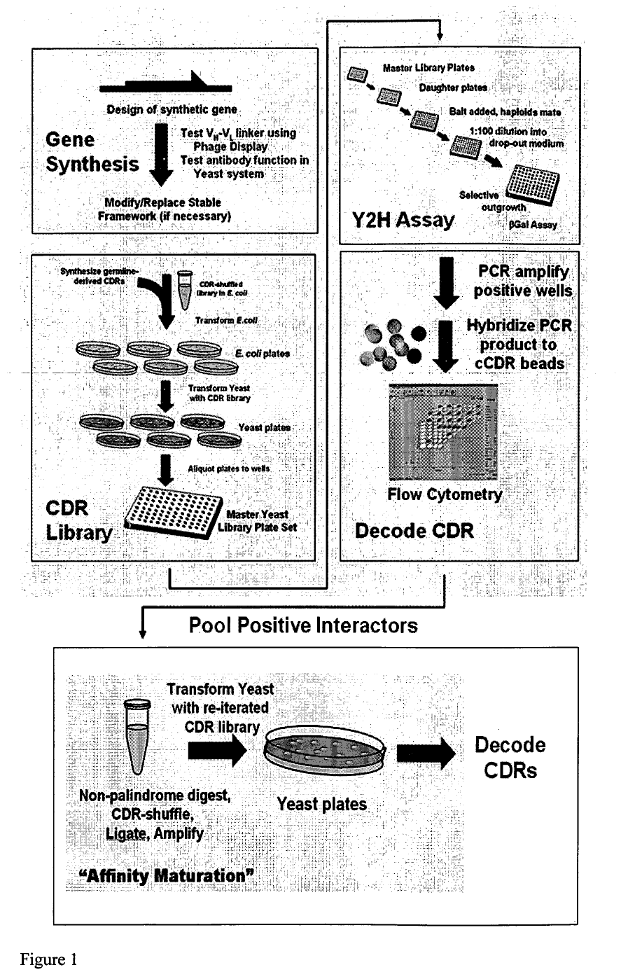

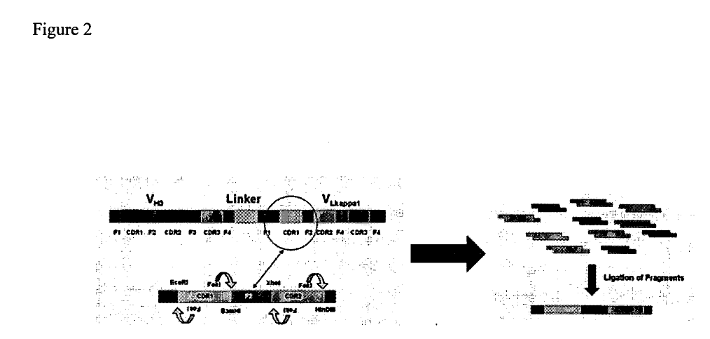

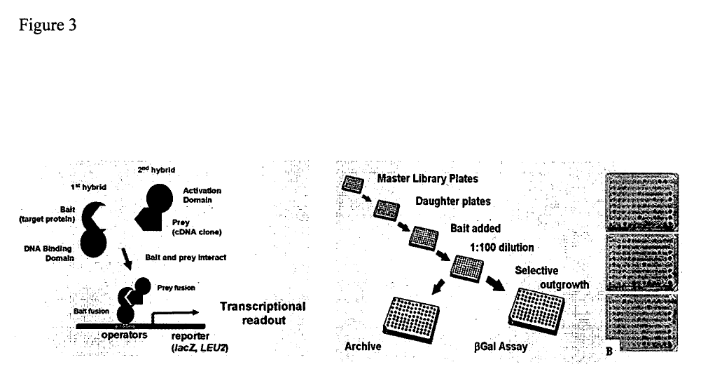

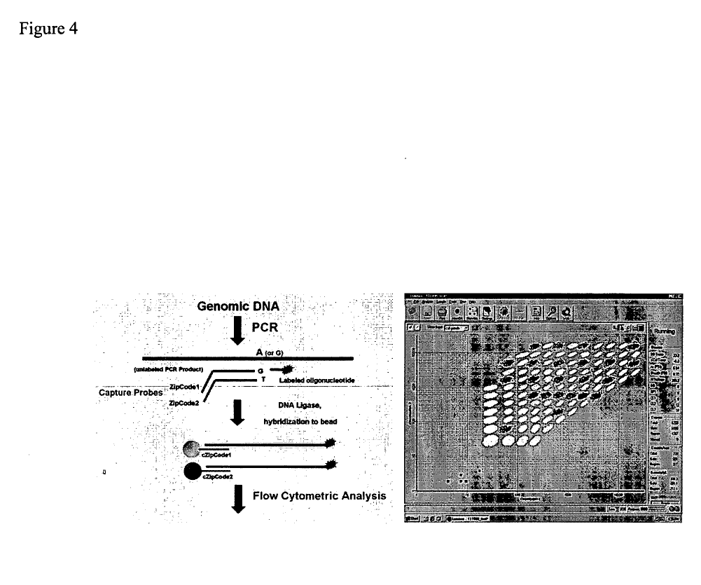

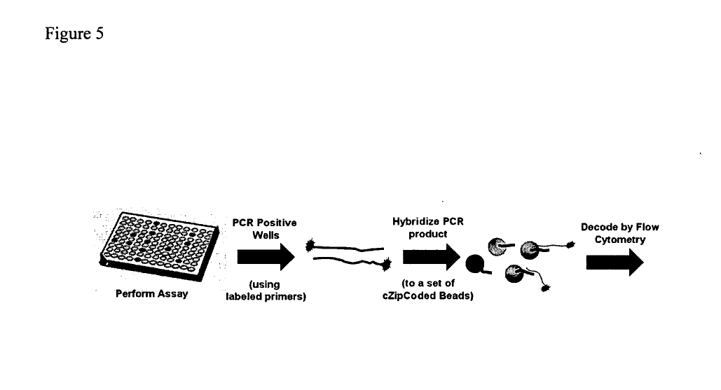

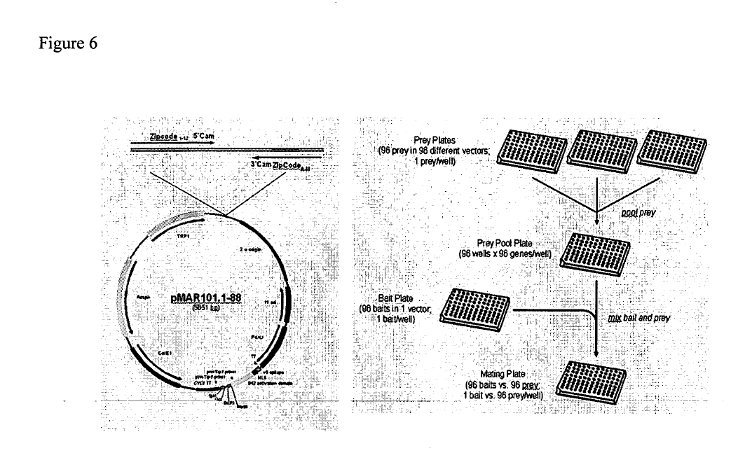

### DETAILED DESCRIPTION OF THE INVENTION

The invention is based in part on the discovery that an in vitro antibody library with a complexity approaching the in vivo antibody repertoire diversity can be created by recombining 10-100 known germline CDRs into each of the six CDR regions. Specifically, a priori-designed germline derived CDRs are gene shuffled to create an initial antibody library complexity of 10⁶-10^{12.} Moreover, using an sutomated system of identification of each of the six CDRs, subsequent rounds of gene shuffling are used to affinity mature the first- round of antibodes resulting in a antibody library with greater than 10¹² complexity. Thus, the invention provides a method to produce and identify an antibody specific for any antigen in an extremely high throughput and cost effective manner.

The basic structure of all antibody or immunoglobulin (Ig) molecules consists of 4 protein chains linked by disulphide bonds. There are two pairs of chains in the molecule: heavy and light. There are two classes (isotypes) of the light chain called kappa and lambda. Heavy chains have five different isotypes which divide the Igs into five different classes, each with different effector functions (in humans IgG1-4, IgA1-2, IgD, IgM, IgE). Each class of heavy chain can combine with either of the light chains. Each chain is divided into regions or domains consisting of around 110 amino acid residues. The light chain has two domains and the heavy has four. The N-terminal domain on both the heavy and light chain are known to be variable in amino acid sequence composition and are thus called variable domains (VL and VH). The other domains are called constant for a similar reason (CL, CH1, CH2, CH3). The variable domains show three regions of hypervariability in sequence called the complementarity determining regions (CDRs). They differ in length and sequence between different antibodies and are mainly responsible for the specificity (recognition) and affinity (binding) of the antibodies to their target markers. Proteolytic digestion of antibodies releases different fragments termed Fv (Fragment variable), Fab (Fragment antigen binding) and Fc (Fragment crystallisation). Although the two domains of the Fv fragment are coded for by separate genes, it has been proven possible to make a synthetic linker that enables the domains to be made as a single protein chain (known as a single chain Fv (scFv) by recombinant methods.

The immune system has evolved to possess the capability of responding to any conceivable antigen. The immune system can do this because both B-lymphocytes and T-lymphocytes have evolved a unique system of gene-splicing called gene translocation a type of gene-shuffling process where various different genes along a chromosome move and join with other genes from the chromosome. The variable heavy chain portion of the Fab is coded for by a combination of 3 genes, called VH, DH, and JH. The variable light chain portion of the Fab consists of either a kappa chain or a lambda chain coded for by a combination of 2 genes, VL and JL. In the DNA of each B-lymphocyte there are multiple forms of each one of these variable determinant genes. Through random gene-splicing, any combination of the multiple forms of each gene can join together resulting in billions of possible gene combinations. Humans produce between 10⁷ and 10⁹ different shaped Fabs. This is known as combinatorial diversity. Additionally, specialized enzymes in the B-lymphocyte cause splicing inaccuracies or add additional nucleotides at the gene junctions to generate further diversity. This is called junctional diversity. Furthermore, as B-lymphocytes proliferate, they undergo affinity maturation a process that “fine tunes” the shape of the epitope binding site. This is because the immunoglobulin V genes of B-lymphocytes have a mutation rate around 100,000 times greater than other cells in the body. This somatic hypermutation creates a great opportunity for selection of variant B-lymphocytes with even better fitting antigen-binding sites that fit the epitope more precisely. The longer and more tightly the antigen binds to the B-cell receptor, the greater the chance that B-lymphocyte has of surviving and replicating. In other words, the “fit” of the antibody can be improved over time.

Several methods have been used for creating antibody diversity in vitro using phage display libraries. These include; i) cloning cDNAs of the immune regions (a native approach), of either immunized or non-immunized cells (to create a naïve library), ii) total synthesis of antibody CDR gene fragments with mixed-nucleotide synthesis, and iii) a semi-synthetic approach whereby a framework gene is synthesized, and the diversity is generated by cloning in a multitude of CDRs, (semi-synthetic approach).

The use of in vivo-formed and proofread gene segments (i.e., naturally-occurring framework genes used as starting point for gene library diversity) has several advantages compared to an in vitro-designed and constructed synthetic oligonucleotides employed in the creation of diverse gene libraries. Proofread segments are optimized with regard to functionality and will most likely not encode antigenic T-cell epitopes, since they have been adapted and processed by the immune system. However, the current techniques are limited by reliance on cDNA. cDNA is produced from immunoglobulin-secreting human B cells and individual CDR libraries are created by PCR. These libraries are mixed with oligonucleotides encoding framework regions (FR1-4) and intact genes encoding variable light and heavy domains are synthesized, using overlap-extension PCR. The synthesized variable light and heavy domains are joined together to form a complete gene encoding scFv antibody fragments. Moreover, the diversity of the current technigues are limited since only six CDRs are shuffled withinin the gene. In contrast, the methods of the invention not only provide for a limitless number of CDRs that can be shuffled but the positions of the individual CDRs are maintained. For example, CDR1 is always at the position of CDR1 in a naturally occuring gene.

Accordingly, the invention provides methods for the production of a fully synthetic single chain antibody gene that can achieve 10¹² complexity with only 100 different CDR's at each of 6 positions. In addition, by using CDR sequences that occur naturally in the human repertoire increases the chance of deriving non-immunogenic antibodies. The synthetic CDRs were initially designed to encode germ-line protein sequences, but they can also be based on artificial CDRs known to bind to specific proteins, for example those CDRs derived by phage display and subsequently analyzed for immunogenicity in humans. The synthetic gene are produced by gene-shuffling using a ‘nonpalindromic recognition sequence’ shuffling scheme. Gene shuffling is done in a way to preserve the location of each CDR, and allows the identification of the strongest binding CDRs to be achieved without the need to have all of the combinations present in the original screen. This solves one of the most important bottlenecks in the generation of antibodies, and mimics natures own solution to the antibody diversity problem. CDRs are chosen with at least one internal amino-acid sequence which can recode a non-palindrome site. Binding of CDRs occur somewhat independent of each other, so the use of this schema creates a library of maximum diversity. The 10-100 CDRs for each of the six CDRs can be shuffled using these non-palindrome enzymes to create a 10⁶-10¹² CDR library. Alternatively, pairs of CDR-bracketing palindrome restriction enzymes (for example, HinDIII and XhoI) are used to shuffle one particular CDR.

Briefly, a synthetic scFv gene comprised of a V_{H}-(linker)-V_{L} gene encoding six CDRs is synthesized, and the gene products are tested for both in vitro (i.e., phage display) and in vivo (i.e., Y2H assay) binding activity to an appropriate antigen. For example, the primary CDR library is transformed into E. coli [note that the clones are amplified on solid medium in E. coli and yeast to reduce clonal expansion (i.e., bias) of any one particular clone in the library]. Plasmid preps from the transformed E. coli are transformed into haploid yeast. The transformed yeast are pooled into wells to create a ‘Master Library Plate Set.’ Wells exhibiting high background (for example, when mated to empty bait-vector) will be eliminated from the Y2H assay at this step. The master library plates are aliquoted to create daughter plates, which are used in an all liquid-format Y2H assay. The antibody gene from wells exhibiting high levels of β galactosidase activity are PCR-amplified with dye-labeled primers, and the resulting fragment hybridized to a set of beads containing oligonucleotides complementary to the CDRs (cCDRs) that were used to create the library. The bead set is analyzed using flow cytometry to decode the amplified CDRs. For affinity maturing the antibodies, a pool of positive clones is digested with the non-palindrome enzyme and the digested fragments re-ligated with T4 ligase. The ligation mixture will be amplified using 5′ and 3′ primers (encoding additionally regions of homology to the prey vector) and recombined into the yeast prey vector by homologous recombination. By titrating the number of LexA DNA binding sites upstream of the reporter genes, the Y2H system is set-up to allow colonies on a plate to give a rough indication of the strength of the binding interaction. Resulting colonies will be assayed first by β gal activity and then decoded as described for the first round. Alternatively pooled positive reshuffled genes are cloned into an E. coli system.

Definitions

DIGESTION of DNA refers to catalytic cleavage of the DNA with a restriction enzyme that acts only at certain sequences in the DNA. The various restriction enzymes referred to herein are commercially available and their reaction conditions, cofactors and other requirements for use are known and routine to the skilled artisan. For analytical purposes, typically, 1 mg of plasmid or DNA fragment is digested with about 2 units of enzyme in about 20 ml of reaction buffer. For the purpose of isolating DNA fragments for plasmid construction, typically 5 to 50 mg of DNA are digested with 20 to 250 units of enzyme in proportionately larger volumes. Appropriate buffers and substrate amounts for particular restriction enzymes are described in standard laboratory manuals, such as those referenced below, and they are specified by commercial suppliers. Incubation times of about 1 hour at 37° C. are ordinarily used, but conditions may vary in accordance with standard procedures, the supplier's instructions and the particulars of the reaction. After digestion, reactions may be analyzed, and fragments may be purified by electrophoresis through an agarose or polyacrylamide gel, using well known methods that are routine for those skilled in the art.

GENETIC ELEMENT generally means a polynucleotide comprising a region that encodes a polypeptide or a region that regulates transcription or translation or other processes important to expression of the polypeptide in a host cell, or a polynucleotide comprising both a region that encodes a polypeptide and a region operably linked thereto that regulates expression. Genetic elements may be comprised within a vector that replicates as an episomal element; that is, as a molecule physically independent of the host cell genome. They may be comprised within mini-chromosomes, such as those that arise during amplification of transfected DNA by methotrexate selection in eukaryotic cells. Genetic elements also may be comprised within a host cell genome;-not in their natural state but, rather, following manipulation such as isolation, cloning and introduction into a host cell in the form of purified DNA or in a vector, among others.

ISOLATED means altered “by the hand of man” from its natural state; i.e., that, if it occurs in nature, it has been changed or removed from its original environment, or both. For example, a naturally occurring polynucleotide or a polypeptide naturally present in a living animal in its natural state is not “isolated,” but the same polynucleotide or polypeptide separated from the coexisting materials of its natural state is “isolated”, as the term is employed herein. For example, with respect to polynucleotides, the term isolated means that it is separated from the chromosome and cell in which it naturally occurs. As part of or following isolation, such polynucleotides can be joined to other polynucleotides, such as DNAs, for mutagenesis, to form fusion proteins, and for propagation or expression in a host, for instance. The isolated polynucleotides, alone or joined to other polynucleotides such as vectors, can be introduced into host cells, in culture or in whole organisms. Introduced into host cells in culture or in whole organisms, such DNAs still would be isolated, as the term is used herein, because they would not be in their naturally occurring form or environment. Similarly, the polynucleotides and polypeptides may occur in a composition, such as a media formulations, solutions for introduction of polynucleotides or polypeptides, for example, into cells, compositions or solutions for chemical or enzymatic reactions, for instance, which are not naturally occurring compositions, and, therein remain isolated polynucleotides or polypeptides within the meaning of that term as it is employed herein.

LIGATION refers to the process of forming phosphodiester bonds between two or more polynucleotides, which most often are double stranded DNAs. Techniques for ligation are well known to the art and protocols for ligation are described in standard laboratory manuals and references, such as, for instance, Sambrook et al., MOLECULAR CLONING, A LABORATORY MANUAL, 2nd Ed.; Cold Spring Harbor Laboratory Press, Cold Spring Harbor, N.Y. (1989) and Maniatis et al., pg. 146, as cited below.

OLIGONUCLEOTIDE(S) refers to relatively short polynucleotides. Often the term refers to single-stranded deoxyribonucleotides, but it can refer as well to single-or double-stranded ribonucleotides, RNA:DNA hybrids and double-stranded DNAs, among others. Oligonucleotides, such as single-stranded DNA probe oligonucleotides, often are synthesized by chemical methods, such as those implemented on automated oligonucleotide synthesizers. However, oligonucleotides can be made by a variety of other methods, including in vitro recombinant DNA-mediated techniques and by expression of DNAs in cells and organisms. Initially, chemically synthesized DNAs typically are obtained without a 5′ phosphate. The 5′ ends of such oligonucleotides are not substrates for phosphodiester bond formation by ligation reactions that employ DNA ligases typically used to form recombinant DNA molecules. Where ligation of such oligonucleotides is desired, a phosphate can be added by standard techniques, such as those that employ a kinase and ATP. The 3′ end of a chemically synthesized oligonucleotide generally has a free hydroxyl group and, in the presence of a ligase, such as T4 DNA ligase, readily will form a phosphodiester bond with a 5′ phosphate of another polynucleotide, such as another oligonucleotide. As is well known, this reaction can be prevented selectively, where desired, by removing the 5′ phosphates of the other polynucleotide(s) prior to ligation.

PLASMIDS generally are designated herein by a lower case p preceded and/or followed by capital letters and/or numbers, in accordance with standard naming conventions that are familiar to those of skill in the art. Starting plasmids disclosed herein are either commercially available, publicly available on an unrestricted basis, or can be constructed from available plasmids by routine application of well known, published procedures. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

POLYNUCLEOTIDE(S) generally refers to any polyribonucleotide or polydeoxribonucleotide, which may be unmodified RNA or DNA or modified RNA or DNA. Thus, for instance, polynucleotides as used herein refers to, among others, single-and double-stranded DNA, DNA that is a mixture of single-and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

POLYPEPTIDES, as used herein, includes all polypeptides as described below. The basic structure of polypeptides is well known and has been described in innumerable textbooks and other publications in the art. In this context, the term is used herein to refer to any peptide or protein comprising two or more amino acids joined to each other in a linear chain by peptide bonds. As used herein, the term refers to both short chains, which also commonly are referred to in the art as peptides, oligopeptides and oligomers, for example, and to longer chains, which generally are referred to in the art as proteins, of which there are many types. It will be appreciated that polypeptides often contain amino acids other than the 20 amino acids commonly referred to as the 20 naturally occurring amino acids, and that many amino acids, including the terminal amino acids, may be modified in a given polypeptide, either by natural processes, such as processing and other post-translational modifications, but also by chemical modification techniques which are well known to the art. Even the common modifications that occur naturally in polypeptides are too numerous to list exhaustively here, but they are well described in basic texts and in more detailed monographs, as well as in a voluminous research literature, and they are well known to those of skill in the art. Among the known modifications which may be present in polypeptides of the present are, to name an illustrative few, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Such modifications are well known to those of skill and have been described in great detail in the scientific literature. Several particularly common modifications, glycosylation, lipid attachment, sulfation, gamma-arboxylation of glutamic acid residues, hydroxylation and ADP-ribosylation, for instance, are described in most basic texts, such as, for instance PROTEINS—STRUCTURE AND MOLECULAR PROPERTIES, 2nd Ed., T. E. Creighton, W. H. Freeman and Company, New York (1993). Many detailed reviews are available on this subject, such as, for example, those provided by Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Analysis for protein modifications and nonprotein cofactors, Meth. Enzymol. 182: 626-646 (1990) and Rattan et al., Protein Synthesis: Posttranslational Modifications and Aging, Ann. N.Y. Acad. Sci. 663: 48-62 (1992). It will be appreciated, as is well known and as noted above, that polypeptides are not always entirely linear. For instance, polypeptides may be branched as a result of ubiquitination, and they may be circular, with or without branching, generally as a result of posttranslation events, including natural processing event and events brought about by human manipulation which do not occur naturally. Circular, branched and branched circular polypeptides may be synthesized by non-translation natural process and by entirely synthetic methods, as well. Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well. For instance, the amino terminal residue of polypeptides made in E. coli, prior to proteolytic processing, almost invariably will be N-formylmethionine. The modifications that occur in a polypeptide often will be a function of how it is made. For polypeptides made by expressing a cloned gene in a host, for instance, the nature and extent of the modifications in large part will be determined by the host cell posttranslational modification capacity and the modification signals present in the polypeptide amino acid sequence. For instance, as is well known, glycosylation often does not occur in bacterial hosts such as E. coli. Accordingly, when glycosylation is desired, a polypeptide should be expressed in a glycosylating host, generally a eukaryotic cell. Insect cell often carry out the same posttranslational glycosylations as mammalian cells and, for this reason, insect cell expression systems have been developed to express efficiently mammalian proteins having native patterns of glycosylation, inter alia. Similar considerations apply to other modifications. It will be appreciated that the same type of modification may be present in the same or varying degree at several sites in a given polypeptide. Also, a given polypeptide may contain many types of modifications. In general, as used herein, the term polypeptide encompasses all such modifications, particularly those that are present in polypeptides synthesized by expressing a polynucleotide in a host cell.

VARIANT(S) of polynucleotides or polypeptides, as the term is used herein, are polynucleotides or polypeptides that differ from a reference polynucleotide or polypeptide, respectively. Variants in this sense are described below and elsewhere in the present disclosure in greater detail. (1) A polynucleotide that differs in nucleotide sequence from another, reference polynucleotide. Generally, differences are limited so that the nucleotide sequences of the reference and the variant are closely similar overall and, in many regions, identical. As noted below, changes in the nucleotide sequence of the variant may be silent. That is, they may not alter the amino acids encoded by the polynucleotide. Where alterations are limited to silent changes of this type a variant will encode a polypeptide with the same amino acid sequence as the reference. Also as noted below, changes in the nucleotide sequence of the variant may alter the amino acid sequence of a polypeptide encoded by the reference polynucleotide. Such nucleotide changes may result in amino acid substitutions, additions, deletions, fusions and truncations in the polypeptide encoded by the reference sequence, as discussed below. (2) A polypeptide that differs in amino acid sequence from another, reference polypeptide. Generally, differences are limited so that the sequences of the reference and the variant are closely similar overall and, in many region, identical. A variant and reference polypeptide may differ in amino acid sequence by one or more substitutions, additions, deletions, fusions and truncations, which may be present in any combination.

Production of Single Chain Antibodies

The preparation of single polypeptide chain binding molecules of the Fv region, single-chain Fv molecules, is described in U.S. Pat. No. 4,946,778, which is incorporated herein by reference. In the present invention, single-chain Fv-like molecules are synthesized by encoding a first variable region of the heavy or light chain, followed by one or more linkers to the variable region of the corresponding light or heavy chain, respectively. The selection of appropriate linker(s) between the two variable regions is described in U.S. Pat. No. 4,946,778. Linkers include for example (Gly₄-Ser)₆ or (Gly-Gly-Gly-Gly-Ser)₂. The linker is used to convert the naturally aggregated but chemically separate heavy and light chains into the amino terminal antigen binding portion of a single polypeptide chain, wherein this antigen binding portion will fold into a structure similar to the original structure made of two polypeptide chains and thus retain the ability to bind to the antigen of interest.

Optionally, the nucleotide sequences encoding the variable regions of the heavy and light chains, joined by a sequence encoding a linker, are joined to a nucleotide sequence encoding antibody constant regions. The constant regions are those which permit the resulting polypeptide to form interchain disulfide bonds to form a dimer, and which contain desired effector functions, such as the ability to mediate antibody-dependent cellular cytotoxicity (ADCC). For an immunoglobulin-like molecule of the invention which is intended for use in humans, the constant regions will typically be substantially human to minimize a potential anti-human immune response and to provide approbate effector functions. Manipulation of sequences encoding antibody constant regions is described in the PCT publication of Morrison and Oi, WO 89/07142, which is incorporated herein by reference. In preferred embodiments, the CH1 domain is deleted and the carboxyl end of the second variable region is joined to the amino terminus of CH2 through the hinge region. The Cys residue of the hinge which makes a disulfide bond with a corresponding Cys of the light chain, to hold the heavy and light chains of the native antibody molecule, can be deleted or, preferably, is substituted with, e.g., a Pro residue or the like. Thus, the Cys residues which remain in the hinge region are those which provide disulfide linkage between two heavy chains.

The isolated polynucleotide molecule codes for a single chain immunoglobulin-like polypeptide having binding affinity for a selected antigen. The immunoglobulin-like polypeptide comprises a first polypeptide comprising the binding portion of the light chain variable region of an antibody or substantially all of the light chain variable region; a second polypeptide comprising the binding portion of the heavy chain variable region of an antibody or substantially all of the variable region; at least one peptide linker linking said first and second polypeptides; and optionally a third polypeptide comprising the constant region domains CH2 and CH3. The peptide linker is not necessarily from an antibody, and links the first and second polypeptides into the single chain polypeptide.

The single chain polypeptide encoded by the polynucleotide molecule may comprise, in sequence: (i) an N-terminal polypeptide from the light chain variable region of an antibody; (ii) a peptide linker; (iii) a C-terminal polypeptide from the heavy chain variable region of an antibody. Alternatively, the elements may be arranged in the sequence: (i) an N-terminal polypeptide from the heavy chain variable region of an antibody; (ii) a peptide linker; (iii) a C-terminal polypeptide from the heavy chain variable region of an antibody.

To prepare the polynucleotide sequence of the single-gene encoded immunoglobulin-like molecule, it is possible to utilize synthetic DNA by synthesizing the entire sequence de novo. Alternatively, it is possible to obtain cDNA sequences coding for certain preserved portions of the variable light and heavy chain regions of the desired antibody, and splice them together, by means of the necessary sequence coding for the peptide linker, which sequences are further spliced to sequences encoding the desired heavy chain constant region domains.

The resulting sequences are amplified by utilizing well known cloning vectors and well known hosts. Furthermore, the amplified sequence, after checking for correctness, can be linked to promoter and terminator signals, inserted into appropriate expression vectors, and transformed into hosts such as eukaryotic hosts, preferably mammalian cells which are capable of correcting processing the immunoglobulin-like chains, e.g., the SP2/0-Ag14 murine myeloma cell line. Bacteria, yeasts (or other fungi) or other mammalian cells may also be utilized. Upon expression the single-chain binding protein is allowed to refold in physiological solution, at appropriate conditions of pH, ionic strength, temperature, and redox potential, and assemble as dimers to form the dimeric immunoglobulin-like molecules. These molecules can then be purified by standard separation procedures. These include chromatography in its various different types, e.g., affinity chromatography, known to those of skill in the art.

The thus obtained purified single-chain immunoglobulin-like binding protein is utilized by itself, in detectably labelled form, in immobilized form, or conjugated to drugs or other appropriate therapeutic agents, in diagnostic, imaging, biosensors, purifications, and therapeutic uses and compositions. Essentially all uses envisioned for antibodies or for variable region fragments thereof can be considered for the molecules of the present invention.

Generally, it is possible to utilize the cDNA sequences obtained from the light and heavy chains of the variable region of the original antibody as a starting point. These sequences can then be joined by means of genetic linkers coding for the peptide linker. As noted above, the genetic sequence can be entirely synthesized de novo or fragments of cDNA can be linked together with the synthetic linkers.

A large source of hybridomas and their corresponding monoclonal antibodies are available for the preparation of sequences coding for the H and L chains of the variable region. Most variable regions of antibodies of a given class are in fact quite constant in their three dimensional folding pattern, except for certain specific hypervariable loops. Thus, to choose and determine the specific binding specificity of the single-gene encoded immunoglobulin-like binding protein of the invention it becomes necessary only to define the protein sequence (and thus the underlying genetic sequence) of the hypervariable region. The hypervariable region will vary from binding molecule to molecule, but the remaining domains of the variable region will remain constant for a given class of antibody.

Source mRNA can be obtained from a wide range of hybridomas. See for example the ATCC Catalogue of Cell Lines and Hybridomas, 7th ed., 1992, American Type Culture Collection, 12301 Parklawn Drive, Rockville, Md. 20852. Hybridomas secreting monoclonal antibodies reactive with a wide variety of antigens are listed therein, are available from the collection, and usable in the invention. Of particular interest are hybridomas secreting antibodies which are reactive with tumor associated antigens, viral antigens, bacterial and fungal antigens, lymphocyte and cell adhesion antigens, and the like. These cell lines and others of similar nature can be utilized to copy mRNA coding for the variable region or hypervariable region or one may determine amino acid sequence from the monoclonal antibody itself. The specificity of the antibody to be engineered will be determined by the original selection process. The class of antibody can be determined by criteria known to those skilled in the art, and one need only replace the sequences of the hypervariable regions (or complementary determining regions). The replacement sequences will be derived from either the amino acid sequence or the nucleotide sequence of DNA copies of the mRNA.

A genetic construct comprising the isolated polynucleotide molecule of the single-gene-encoded immunoglobulin-like molecule is typically placed under the control of a single promoter. A variety of promoters and transcriptional enhances suitable for controlling and/or enhancing immunoglobulin expression are available, e.g., the human cytomegalovirus promoter, etc. DNA constructs for expressing human immunoglobulins are described in EP patent publication EP 0 314 161, incorporated herein by reference. The expression of the immunoglobulin-like molecule can also be placed under control of other regulatory sequences which are known to those skilled in the art.

The expressed and refolded single-gene-encoded immunoglobulin-like binding proteins of the invention can be labelled with detectable labels such as radioactive atoms, enzymes, biotin/avidin labels, chromophores, chemiluminescent labels, and the like for carrying out standard immunodiagnostic procedures. These procedures include competitive and immunometric (or sandwich) assays. See., e.g., U.S. Pat. No. 4,376,110, incorporated herein by reference. These assays can be utilized for the detection of antigens in diagnostic samples. In competitive and/or sandwich assays, the binding proteins of the invention can also be immobilized on such insoluble solid phases as beads, test tubes, or other polymeric materials. For imaging procedures, the binding molecules of the invention can be labelled with opacifying agents, such as NMR contrasting agents or X-ray contrasting agents. Methods of binding labelling or imaging agents or proteins as well as binding the proteins to insoluble solid phases are well known in the art. The dimeric immunoglobulin-like proteins can also be used for therapy when labeled or coupled to enzymes or toxins, and for purification of products, especially those produced by the biotechnology industry, or can be used unlabeled. Thus, uses for the single-gene-encoded immunoglobulin-like binding proteins of the invention include both in vitro diagnostic assays and in vivo diagnostic assays (diagnostic imaging). Such uses are discussed in detail in co-pending U.S. application Ser. No. 07/547,336, which is incorporated herein by reference.

Vectors, Host Cells, and Expression

The present invention also relates to vectors which include polynucleotides of the present invention, host cells which are genetically engineered with vectors of the invention and the production of polypeptides of the invention by recombinant techniques. Host cells are genetically engineered to incorporate polynucleotides and express polypeptides of the present invention. For instance, polynucleotides are introduced into host cells using well known techniques of infection, transduction, transfection, transvection and transformation. The polynucleotides are\introduced alone or with other polynucleotides. Such other polynucleotides are introduced independently, co-introduced or introduced joined to the polynucleotides of the invention. Thus, for instance, polynucleotides of the invention may be transfected into host cells with another, separate, polynucleotide encoding a selectable marker, using standard techniques for co-transfection and selection in, for instance, mammalian cells. In this case the polynucleotides generally will be stably incorporated into the host cell genome. Alternatively, the polynucleotides are joined to a vector containing a selectable marker for propagation in a host. The vector construct are introduced into host cells by the aforementioned techniques. Generally, a plasmid vector is introduced as DNA in a precipitate, such as a calcium phosphate precipitate, or in a complex with a charged lipid. Electroporation also may be used to introduce polynucleotides into a host. If the vector is a virus, it is packaged in vitro or introduced into a packaging cell and the packaged virus may be transduced into cells.

A wide variety of techniques suitable for making polynucleotides and for introducing polynucleotides into cells in accordance with this aspect of the invention are well known and routine to those of skill in the art. Such techniques are reviewed at length in Sambrook et al. cited above, which is illustrative of the many laboratory manuals that detail these techniques.

In accordance with this aspect of the invention the vector is, for example, a plasmid vector, a single or double-stranded phage vector, a single or double-stranded RNA or DNA viral vector. Such vectors may be introduced into cells as polynucleotides, preferably DNA, by well known techniques for introducing DNA and RNA into cells. The vectors, in the case of phage and viral vectors also may be and preferably are introduced into cells as packaged or encapsidated virus by well known techniques for infection and transduction. Viral vectors may be replication competent or replication defective. In the latter case viral propagation generally will occur only in complementing host cells. Preferred among vectors, in certain respects, are those for expression of polynucleotides and polypeptides of the present invention. Generally, such vectors comprise cis-acting control regions effective for expression in a host operatively linked to the polynucleotide to be expressed. Appropriate trans-acting factors either are supplied by the host, supplied by a complementing vector or supplied by the vector itself upon introduction into the host. In certain preferred embodiments in this regard, the vectors provide for specific expression. Such specific expression may be inducible expression or expression only in certain types of cells or both inducible and cell-specific. Particularly preferred among inducible vectors are vectors that can be induced for expression by environmental factors that are easy to manipulate, such as temperature and nutrient additives. A variety of vectors suitable to this aspect of the invention, including constitutive and inducible expression vectors for use in prokaryotic and eukaryotic hosts, are well known and employed routinely by those of skill in the art.

The engineered host cells are cultured in conventional nutrient media, which may be modified as appropriate for, inter alia, activating promoters, selecting transformants or amplifying genes. Culture conditions, such as temperature, pH and the like, previously used with the host cell selected for expression generally will be suitable for expression of polypeptides of the present invention as will be apparent to those of skill in the art.

A great variety of expression vectors can be used to express a polypeptide of the invention. Such vectors include chromosomal, episomal and virus-derived vectors e.g., vectors derived from bacterial plasmids, from bacteriophage, from yeast episomes, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids, all may be used for expression in accordance with this aspect of the present invention. Generally, any vector suitable to maintain, propagate or express polynucleotides to express a polypeptide in a host may be used for expression in this regard. Preferably, the vector is a multifunctional vector that allows expression of the polypeptide in multiple cell types. Particularly suitable multifunction vectors are described below.

The appropriate DNA sequence is inserted into the vector by any of a variety of well-known and routine techniques. In general, a DNA sequence for expression is joined to an expression vector by cleaving the DNA sequence and the expression vector with one or more restriction endonucleases and then joining the restriction fragments together using T4 DNA ligase. Procedures for restriction and ligation that can be used to this end are well known and routine to those of skill. Suitable procedures in this regard, and for constructing expression vectors using alternative techniques, which also are well known and routine to those skill, are set forth in great detail in Sambrook et al. cited elsewhere herein.

The DNA sequence in the expression vector is operatively linked to appropriate expression control sequence(s), including, for instance, a promoter to direct mRNA transcription. Representatives of such promoters include the phage lambda PL promoter, the E. coli lac, trp and tac promoters, the SV40 early and late promoters and promoters of retroviral LTRs, to name just a few of the well-known promoters. It will be understood that numerous promoters not mentioned are suitable for use in this aspect of the invention are well known and readily may be employed by those of skill in the manner illustrated by the discussion and the examples herein. In general, expression constructs will contain sites for transcription initiation and termination, and, in the transcribed region, a ribosome binding site for translation. The coding portion of the mature transcripts expressed by the constructs will include a translation initiating AUG at the beginning and a termination codon appropriately positioned at the end of the polypeptide to be translated. In addition, the constructs contain control regions that regulate as well as engender expression. Generally, in accordance with many commonly practiced procedures, such regions will operate by controlling transcription, such as repressor binding sites and enhancers, among others. Vectors for propagation and expression generally will include selectable markers. Such markers also may be suitable for amplification or the vectors may contain additional markers for this purpose. In this regard, the expression vectors preferably contain one or more selectable marker genes to provide a phenotypic trait for selection of transformed host cells. Preferred markers include dihydrofolate reductase or neomycin resistance for eukaryotic cell culture, and tetracycline, theomycin, kanamycin or ampicillin resistance genes for culturing E. coli and other bacteria.

The vector containing the appropriate DNA sequence as described elsewhere herein, as well as an appropriate promoter, and other appropriate control sequences, is introduced into an appropriate host using a variety of well known techniques suitable to expression therein of a desired polypeptide. Representative examples of appropriate hosts include bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Hosts for of a great variety of expression constructs are well known, and those of skill will be enabled by the present disclosure readily to select a host for expressing a polypeptides in accordance with this aspect of the present invention. Various mammalian cell culture systems can be employed for expression, as well. Examples of mammalian expression systems include the COS-7 lines of monkey kidney fibroblast, described in Gluzman et al., Cell 23: 175 (1981). Other cell lines capable of expressing a compatible vector include for example, the C127, 3T3, CHO, HeLa, human kidney 293 and BHK cell lines. More particularly, the present invention also includes recombinant constructs, such as expression constructs, comprising one or more of the sequences described above. The constructs comprise a vector, such as a plasmid or viral vector, into which such a sequence of the invention has been inserted. The sequence may be inserted in a forward or reverse orientation. In certain preferred embodiments in this regard, the construct further comprises regulatory sequences, including, for example, a promoter, operably linked to the sequence. Large numbers of suitable vectors and promoters are known to those of skill in the art, and there are many commercially available vectors suitable for use in the present invention. The following vectors, which are commercially available, are provided by way of example. Among vectors preferred for use in bacteria are pHE4-5 (ATCC Accession No. 209311; and variations thereof), pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia Among preferred eukaryotic vectors are pWLNEO, pSV2CAT, pOG44, pXT1 and pSG available from Stratagene; and pSVK3, pBPV, pMSG and pSVL available from Pharmacia. These vectors are listed solely by way of illustration of the many commercially available and well known vectors that are available to those of skill in the art for use in accordance with this aspect of the present invention.

It will be appreciated that any other plasmid or vector suitable for, for example, introduction, maintenance, propagation or expression of a polynucleotide or polypeptide of the invention in a host may be used in this aspect of the invention. Promoter regions are selected from any desired gene using vectors that contain a reporter transcription unit lacking a promoter region, such as a chloramphenicol acetyl transferase (“cat”) transcription unit, downstream of restriction site or sites for introducing a candidate promoter fragment; i.e., a fragment that may contain a promoter. As is well known, introduction into the vector of a promoter-containing fragment at the restriction site upstream of the cat gene engenders production of CAT activity, which can be detected by standard CAT assays. Vectors suitable to this end are well known and readily available. Two such vectors are pKK232-8 and pCM7. Thus, promoters for expression of polynucleotides of the present invention include not only well known and readily available promoters, but also promoters that readily may be obtained by the foregoing technique, using a reporter gene. Among known bacterial promoters suitable for expression of polynucleotides and polypeptides in accordance with the present invention are the E. coli lacI and lacZ promoters, the T3 and T7 promoters, the T5 tac promoter, the lambda PR, PL promoters and the trp promoter. Among known eukaryotic promoters suitable in this regard are the CMV immediate early promoter, the HSV thymidine kinase promoter, the early and late SV40 promoters, the promoters of retroviral LTRs, such as those of the Rous sarcoma virus (“RSV”), and metallothionein promoters, such as the mouse metallothionein-I promoter. Selection of appropriate vectors and promoters for expression in a host cell is a well known procedure and the requisite techniques for expression vector construction, introduction of the vector into the host and expression in the host are routine skills in the art. Generally, recombinant expression vectors will include origins of replication, a promoter derived from a highly-expressed gene to direct transcription of a downstream structural sequence, and a selectable marker to permit isolation of vector containing cells after exposure to the vector.

The present invention also relates to host cells containing the above-described constructs discussed above. The host cell can be a higher eukaryotic cell, such as a mammalian cell, or a lower eukaryotic cell, such as a yeast cell, or the host cell can be a prokaryotic cell, such as a bacterial cell. Constructs in host cells can be used in a conventional manner to produce the gene product encoded by the recombinant sequence. Alternatively, the polypeptides of the invention can be synthetically produced by conventional peptide synthesizers. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook et al., MOLECULAR CLONING: A LABORATORY MANUAL, 2nd Ed., Cold Spring Harbor Laboratory Press, Cold Spring Harbor, N.Y. (1989). Transcription of the DNA encoding the polypeptides of the present invention by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at bp 100 to 270, the cytomegalovirus early promoter enhancer, the polyoma enhancer on the late side of the replication origin, and adenovirus enhancers. Polynucleotides of the invention, encoding the heterologous structural sequence of a polypeptide of the invention generally will be inserted into the vector using standard techniques so that it is operably linked to the promoter for expression. The polynucleotide will be positioned so that the transcription start site is located appropriately 5′ to a ribosome binding site. The ribosome binding site will be 5′ to the AUG that initiates translation of the polypeptide to be expressed. Generally, there will be no other open reading frames that begin with an initiation codon, usually AUG, and lie between the ribosome binding site and the initiating AUG. Also, generally, there will be a translation stop codon at the end of the polypeptide and there will be a polyadenylation signal and a transcription termination signal appropriately disposed at the 3′ end of the transcribed region. For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide may be expressed in a modified form, such as a fusion protein, and may include not only secretion signals but also additional heterologous functional regions. Thus, for instance, a region of additional amino acids, particularly charged amino acids, may be added to the N-terminus of the polypeptide to improve stability and persistence in the host cell, during purification or during subsequent handling and storage. Also, region also may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. Following transformation of a suitable host strain and growth of the host strain to an appropriate cell density, where the selected promoter is inducible it is induced by appropriate means (e.g., temperature shift or exposure to chemical inducer) and cells are cultured for an additional period. Cells typically then are harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents, such methods are well know to those skilled in the art.

The a scFV polypeptide can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Well known techniques for refolding protein may be employed to regenerate active conformation when the polypeptide is denatured during isolation and or purification. Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

Multifunctional Vector

The present invention provides a multi-functional vector useful in a yeast two-hybrid (Y2H) system and in a phage display system, and is, optionally, useful for polypeptide expression in E. coli. The present invention removes the limitations of traditional methods for expression of heterologous proteins involving the subcloning a gene of interest into DNA vectors specific for the desired expression host, which generally require either restriction digestion or PCR amplification of the gene, subcloning into an appropriate expression vector, and DNA sequence verification. By multifunctional vector, is meant a vector that allows expression of a polypeptide in two or more species or higher classes of organisms, such as bacteria, yeast, and mammals. The multifunctional vector is a bi-functional vector. Alternatively, the multifunctional vector is a tri-functional vector. The vector is a plasmid vector. Alternatively, the vector is a BAC vector, a cosmid vector, or a YAC vector.

The expression vector of the invention contains a polynucleotide sequence that includes an activation domain, a yeast promoter sequence, a polynucleotide sequence encoding a bacteriophage coat protein or fragment thereof, a bacterial promoter sequence, a protein fusion site, and a suppressible stop codon such as the amber stop codon. Optionally, the expression vector includes one or more of the following polynucleotide sequences: (i) a nuclear localization sequence; (ii) an epitope tag; (iii) a yeast transcription termination signal; (iv) an origin of replication; (v) a selectable marker, e.g. a yeast selectable marker of a bacterial selectable marker; and (vi) an additional promoter. The polynucleotide sequences of the vector are in functional combination. By functional combination is meant that the nucleotide sequence of interest is linked in a manner that allows for expression of the nucleotide sequence.

Suitable promoters for expression in yeast include for example, GAL1, GAL7, GAL 10, ADH1, ADH1 PGK1, ENO, PYK1, PHO5, MET 25, CUP 1, CaMV, GRE, and ARE. Promoters for expression in bacterial include for example, lacI, lacZ, T3, T7, gpt, lambda P_{R}, P_{L} and TRP. Optionally, the vector contains a third promoter, such as a mammalian promoter such as CMV immediate early, HSV thymidine kinase, early and late SV40, a retroviral LTR, elongation factor-1a (EF-1a), and mouse metallothionein-I, an insect promoter, a plant promoter or a viral promoter. This additional promoter may be inserted proximal or distal to the yeast and/or bacterial promoters. The promoters are constitutive, regulated or heterlogous.

Activation domains allow the manipulation of the binding properties of the prey fusion proteins. The activation domain is for example, a B42 activation domain, a VP 16 activation domain, a GAL4 activation domain, and an NF-κB activation domain.

The bacteriophage coat protein is a full length polypeptide, or any fragment thereof sufficient to direct expression of the fusion protein to the surface of the capsid. A The bacteriophage coat protein is a Ml 3 coat protein such as gpIII, gpVII, or gpVIII proteins or fragments are suitable or a 10A capsid protein from the T7 phage.

A nuclear localization sequence (NLS) is for example an SV40 large T antigen nuclear localization sequence, a Matcc2 nuclear localization sequence, a nucleoplasmin nuclear localization sequence, a c-myc nuclear localization sequence or other NLS sequences known in the art.

A yeast selectable marker is optionally provided in order to select for yeast containing the multifunctional vector. A yeast selectable marker includes for example, a TRP1, URA3, HIS3, LEU2, and LYS2. Likewise, a bacterial selection marker is optionally provided in order to select for bacteria containing the multifunctional vector; these selection markers are generally antibiotic resistance genes, such as markers conferring resistance to ampicillin, streptomycin, gentamicin, ofloxacin, tetracycline, kanamycin, spectinomycin, or chloramphenicol.

Further, the vector contains an epitope tag sequence for the detection of the fusion protein. For example the vector contains the a V5 epitope, which allows detection of the fusion protein(s) using an Anti-V5 antibody. Alternatively the epitope tag is a 6-His epitope tag, a c-myc tag, a Flag tag, a GFP tag, a GST tag, a HA tag, a luciferase tag, a Protein C tag, an S-tag, a T7 tag, a thioredoxin tag, and a VSV-g tag.

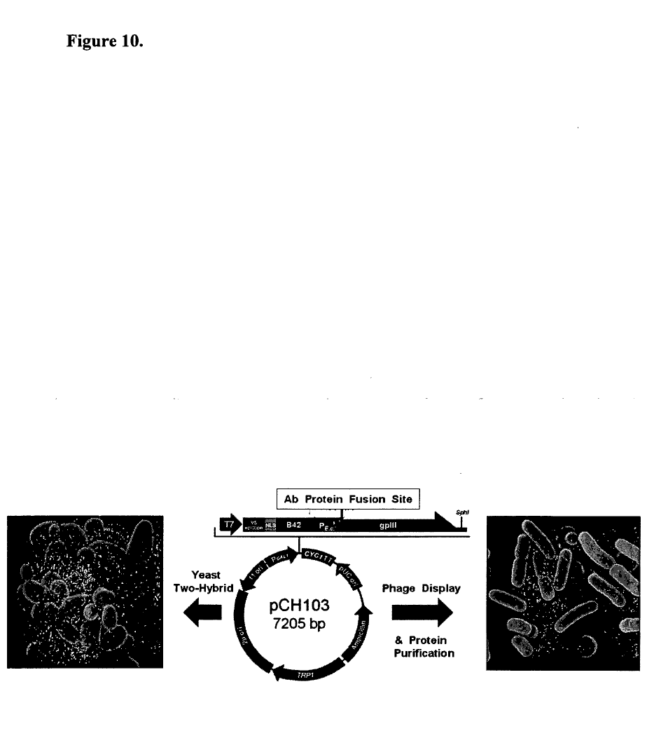

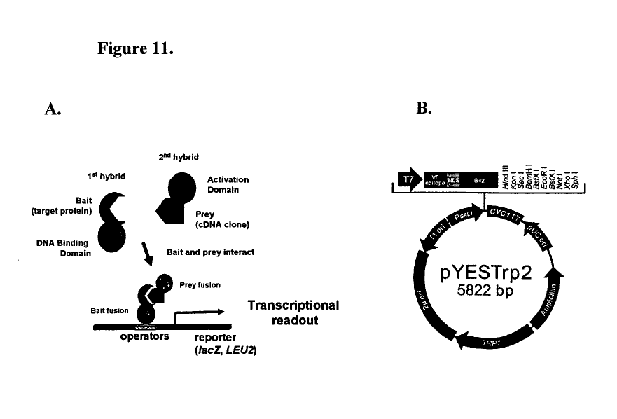

An exemplary expression vector, includes the plasmid vector pCH103, as illustrated schematically in FIG. 10. A nucleotide sequence encoding a polypeptide of interest, such as a single chain antibody or fragment thereof, is inserted into the protein fusion site of the vector, such that a fusion protein is generated when the nucleic acid of the expression vector is expressed in a cell, e.g., yeast or E. coli.

In yeast, the pCH103 vector, containing the nucleotide sequence encoding a polypeptide of interest, expresses in yeast a fusion protein distinct from the fusion protein expressed in E. coli. As shown in FIG. 10, the pCH103 vector contains a GAL1 yeast promoter, a SV40 large T antigen nuclear localization sequence (NLS), a B42 activation domain (AD), a CYC1 transcription termination signal, a TRP1 gene, a 2μ origin, an fl origin, an E. coli lac promoter (P_{E.c.}), an M13 gpIII gene and a protein fusion site. The GAL1 promoter provides for constitutive expression of a fusion protein containing the NLS, the B42 AD, and the polypeptide of interest in, e.g., L40 yeast, and inducible expression in, e.g., EGY48/pSH18-34. The E. coli lac promoter is provided in-frame with the yeast promoter but does not affect the fusion protein. Moreover, the suppressible amber stop codon is not suppressed in yeast, so transcription is terminated upstream of the bacteriophage M113 gpIII sequence. The SV40 NLS directs fusions to the nucleus for potential interaction of the AD-containing fusion protein with LexA fusions (which are encoded by the bait vector). Thus, yeast-expressed fusion proteins provided by the pCH103 vector are useful in the yeast two-hybrid assay.

In E. coli or other bacteria, the nucleotide sequence of the B42 activation domain is not recognized by the bacterial RNA polymerase; thus, the translated fusion protein contains the protein of interest and the M13 phage coat protein, but lacks the B42 activation domain. This fusion protein is useful in phage display assays. Optionally, the PCH103 vector is introduced into a bacterial strain that cannot suppress the amber stop codon, thereby producing recombinant protein of interest lacking the M13 coat protein.

The vector described above contains the B42 activation domain attached at the N-terminus of the protein of interest when expressed in yeast or mammalian cells, while the M13 coat protein is located C-terminal to the protein of interest when expressed in bacteria. In an alternative aspect of the invention, the multifunctional expression vector includes a polynucleotide sequence comprising a yeast promoter sequence, a bacterial promoter sequence in-frame with and downstream of the yeast promoter sequence, a GTG initiation codon downstream of the bacterial promoter sequence, a polynucleotide sequence encoding a bacteriophage coat protein downstream of the initiation codon, wherein the polynucleotide sequence does not encode for an in-frame methionine, an fmet initiation codon downstream of the polynucleotide sequence encoding the bacteriophage coat protein, a protein fusion site downstream of the fmet initiation codon, a suppressible stop codon downstream of the protein fusion site, an activation domain downstream of the suppressible stop codon, and a yeast transcription stop codon downstream of the activation domain. The result of this colocation of elements is that when expressed in yeast, the GTG initiation codon is not recognized by the yeast polymerase and the bacteriophage coat protein lacks any in-frame methionines; thus, the translated fusion protein begins at the fmet codon upstream of the protein of interest and contains the B42 activation domain at the C-terminus. When the pCH103 vector is introduced into a bacteria, the M13 coat protein is at the N-terminus of the fusion protein, and the suppressible stop codon terminates translation prior to the B42 activation domain.

Pharmaceutical Compositions

The scFVs, or nucleic acid molecules encoding these scFVs, (also referred to herein as “Therapeutics” or “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The active agents disclosed herein can also be formulated as liposomes. Liposomes are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No.5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a scFV) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Sustained-release preparations can be prepared, if desired. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The pharmaceutical compositions can be included in a container, pack, or dispenser together with instructions for administration.

The invention will be further illustrated in the following non-limiting examples.

### EXAMPLE 1

### General Methods

Yeast Two-Hybrid Assay

The Y2H method is a technique for identifying the protein-protein interactions that occur within a cell. Y2H is useful to study intracellular proteins and cytoplasm domains of membrane-bound proteins. As shown in Figure X, two-hybrid technology exploits the fact that disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

The active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

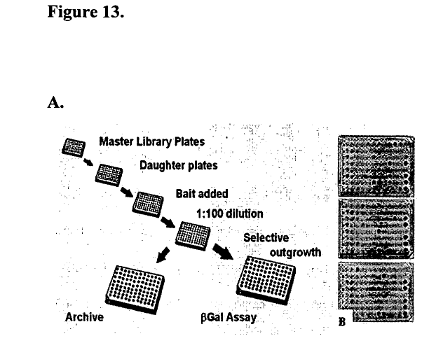

Oral or parenteral compositions are formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit library is aliquoted to daughter plates. The mating mixture is then diluted and the outgrown yeast library is aliquotted to separate 96-well plates, into which is seeded a bait-expressing strain of the opposite mating type. After mating, diploids are subjected to prototrophic selection and assayed for β galactosidase activity. FIG. 13B shows an Y2H analysis of the human nuclear receptor RXRα. Each well of the three 96-well microtiter plates represents β galactosidase assay on prototrophic-selected diploids arising from a pEG-RXRα bait plasmid-expressing yeast strain mated to about 1000 yeast clones of a pJD4.5 AD library. The right-most columns in each plate represent both positive and negative controls. CPRG was used as the chromogenic substrate. Dark-red wells are indicative of the presence of an interaction occurring within the well (See Buckholz et al.).

Liquid mating of yeast will be performed essentially as described in Buckholz [Buckholz, 1999]. To validate this method, 5 μl from each well of the prey yeast antibody library will be transferred into a fresh 96-well V-bottom plate. Bait cultures will be spun down and the cells resuspended in 45 μL YEP galactose+raffinose broth with antibiotics. A 25 μl aliquot of bait culture will be added to the 5 μl of prey culture in each well. Following a 48 hour incubation at 30° C., 200 μl of minimal selective dropout media minus uracil, histidine, tryptophan and leucine, plus 2% galactose and 1% raffinose (SGR-UHWL) will be added to dilute the rich YPD media 1:10. After incubating an additional 48 hours, 5 μl of samples will be transferred to a new microtiter plate and diluted 1:40 using SGR-UHWL to a final volume of 205 μl. The diluted matings will be further incubated for an additional 3-5 days at 30° C. The mating mixture (25 μl) will be transferred to 96-well assay plates and β galactosidase assay will perform as previously described [Buckholz, 1999]. Positive clones will be DNA sequenced.

For high-throughput analysis 175 μl of each prey culture will be transferred to a 50 ml conical centrifuge tube and spun down, the supernatant removed, and the cells resuspended in 45 mL YNB-Trp+glucose with antibiotics. A 5 μl aliquot of each bait culture will be transferred into a fresh 96-well V-bottom plate and 25 μl of the pooled prey culture will be added to each well. Positive clones will be DNA sequenced.

Bacterial Two-Hybrid (B2H) System

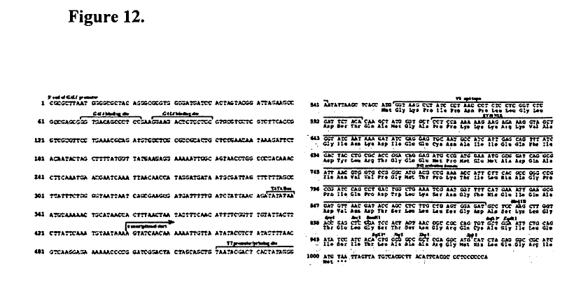

The B2H system is based on a methodology developed by Dove, Joung, and Hochschild of Harvard Medical School and further refined by Joung and Pabo of the Massachusetts Institute of Technology. The B2H system detects protein-protein interactions based on transcriptional activation. As shown in figure x11, the bacterial two-hybrid system uses the transcriptional activators are modular in nature. Two physically distinct functional domains are necessary: a DNA binding domain (DBD) and an activation domain (AD). In the Two-Hybrid System a known gene, X, is cloned into the “bait” vector. In the system developed by Brent, it is expressed as a fusion with the bacterial-derived LexA DBD. A second gene, Y, or a library of cDNAs encoding potential interactors is cloned in frame with the B42 AD. The “bait” and “prey” fusion constructs are transformed into one of sveral available yeast strains. In these yeast strains, the LexA operator is engineered into the 5′ flanking region of reporter genes. The LexA DBD-“bait” fusion will bind specifically to this engineered site. If the X and Y proteins interact, the B42 AD is brought into proximity with the LexA DBD and transcription of the reporter genes is activated. Positive interactors are identified by plating on nutrient deficient medium and screening for β galactosidase activity. These positive clones can be analyzed by restriction analysis, PCR, or sequencing. An exemplary Y2H vector is the pYESTrp2 vector (‘bait’ vector, Invitrogen, Carlsbad, Calif.). An exemplary nucleic acid sequence containing the B42 activation domain and multiple cloning site is shown in FIG. 12 A bait gene is cloned in frame with the sequence encoding the V5 epitope-NLS-B42 fusion protein to create a “prey” fusion protein with a nuclear localization signal, activation domain, and an epitope for detection.

Y2H has been incorporated into high-throughput methodologies useful in the dissect the molecular networks operating in cells, which include high-throughput cloning, genotyping, and yeast two-hybrid analysis [(HT-Y2H), Buckholz, 1999; Nelsen, 2002; Taylor, 2001; Uetz, 2000; Walhout, 2001; Slentz-Kessler, 2000; Uetz P, 2000].

The present invention anticipates the use of automated formats for screening Y2H libraries for protein-protein interaction [Buckholz, 1999]. One such automated format format contains a liquid array in which pooled library subsets of yeast, each expressing up to, e.g., 250, 500, 1000, 1500, 2000, 5000, 10000 or more different cDNAs, are mated to a yeast strain of the opposite mating type. Interactors are detected by a liquid assay for β-galactosidase activity following prototrophic selection. The formatting of the cDNA library lends itself to functional subtraction of the promiscuous interactors. Also, the liquid arrayed format enables electronic handling of the data derived from interaction screening, which, together with the automated handling of the samples, which is advantageous in the use of methods for large proteome analysis and non-random screening approaches. As shown in FIG. 13A, the arrayed cDNA library pooled subsets are created for the ‘Master Library Plates.’ For automated identification of interactors in the arrayed library, each pooled subset of the master transcriptional activation of the 1 operator to show protein-protein interactions. A protein of interest (the bait) is fused to the full-length bacteriophage lambda cI repressor protein (lambda cI, 237 amino acids), which binds to the 1 operator. The target protein is fused to the N-terminal domain of the a-subunit of RNA polymerase (248 amino acids). When the bait and target interact, they recruit and stabilize the binding of RNA polymerase close to the promoter and activate the transcription of the His3 reporter gene. A second reporter gene, aadA, is expressed from the same promoter and confers resistance to streptomycin, providing an additional mechanism to validate the bait and target interaction.

While the Y2H system has been widely and successfully exploited, a method that utilizes E. coli is valuable for many reasons: E. coli grows much faster than yeast, it is transformed with higher efficiency so larger numbers of interactions can be more rapidly and easily screened, and isolating plasmid DNA from E. coli is easier than DNA isolation from yeast. Furthermore, using E. coli for two-hybrid screening reduces the chance that the host harbors a eukaryotic homologue of one of the interacting protein partners. Some eukaryotic regulatory proteins, such as cell cycle checkpoint proteins and signal transduction pathway proteins may be toxic in yeast because they interfere with the function of yeast homologues; presumably they would not be as harmful in E. coli. For the same reason, use of a bacterial system could also reduce the number of false positives observed. Although some heterologous proteins could be toxic to an E. coli host, and bacteria lack the ability to perform some posttranslational modifications, an E. coli two-hybrid system makes for an important experimental alternative.

Phage Display

M13 Phage Display

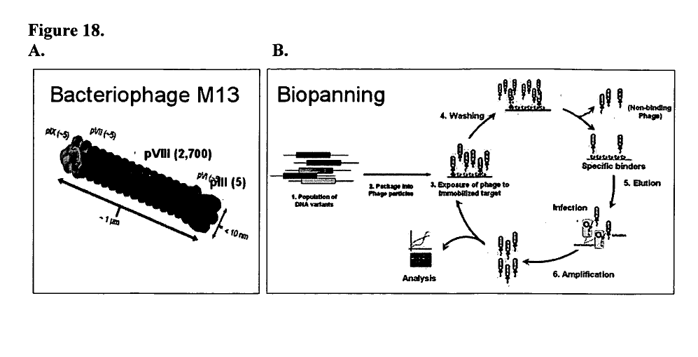

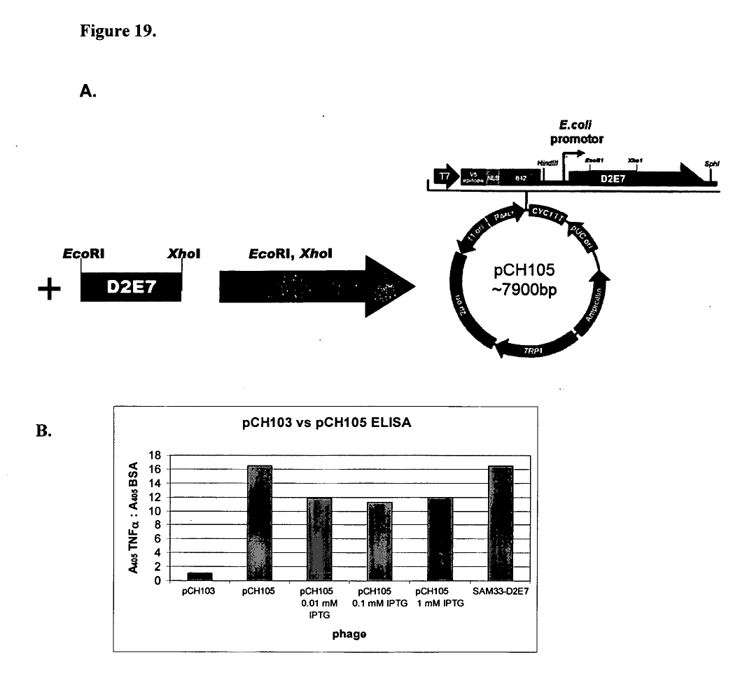

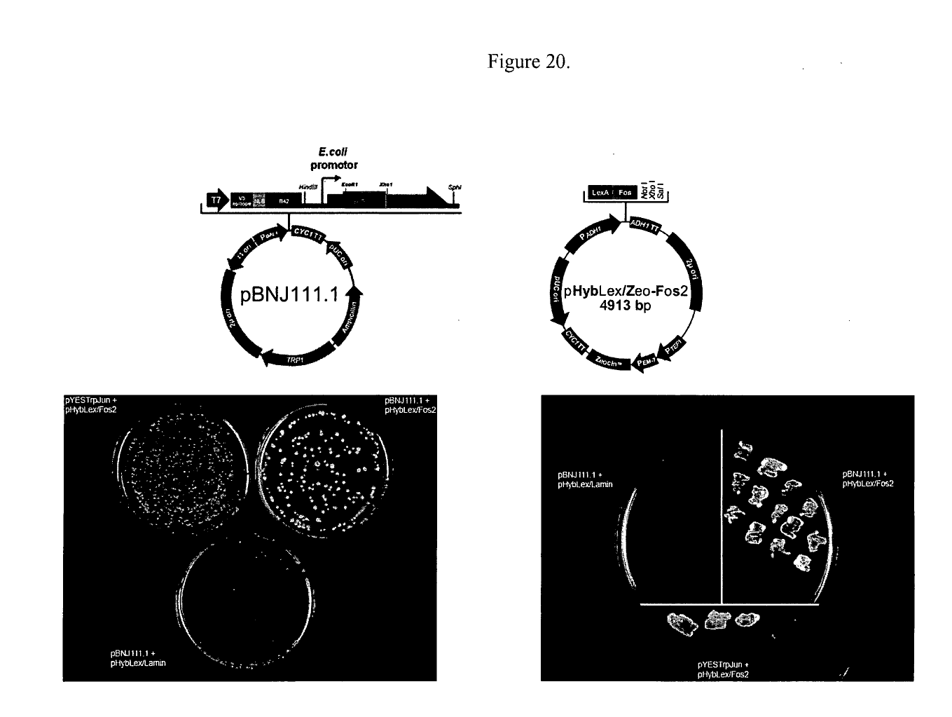

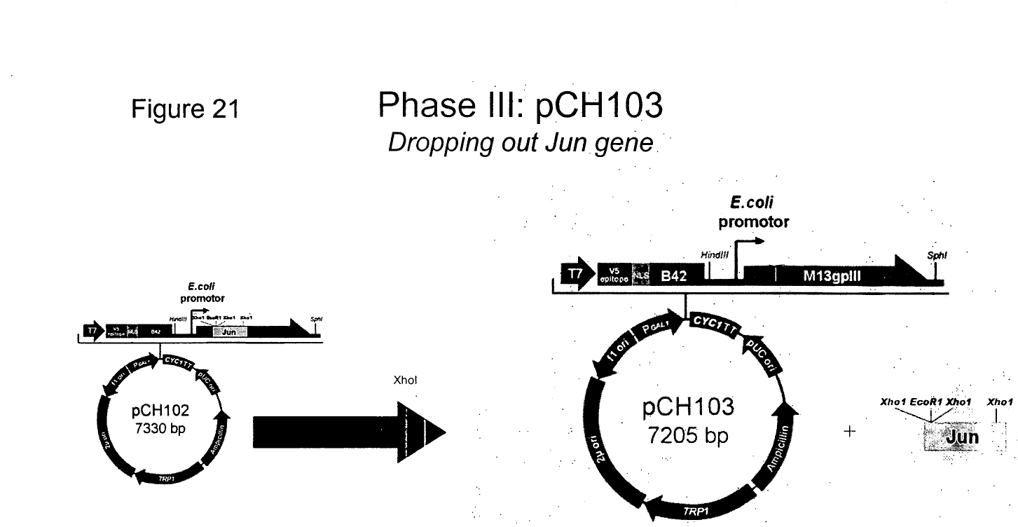

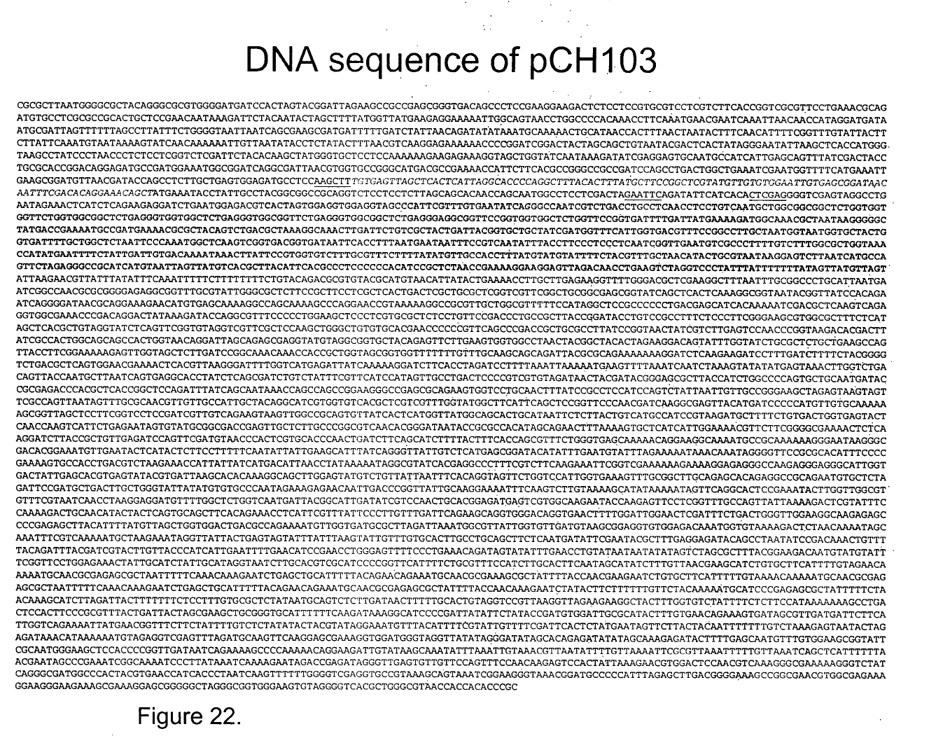

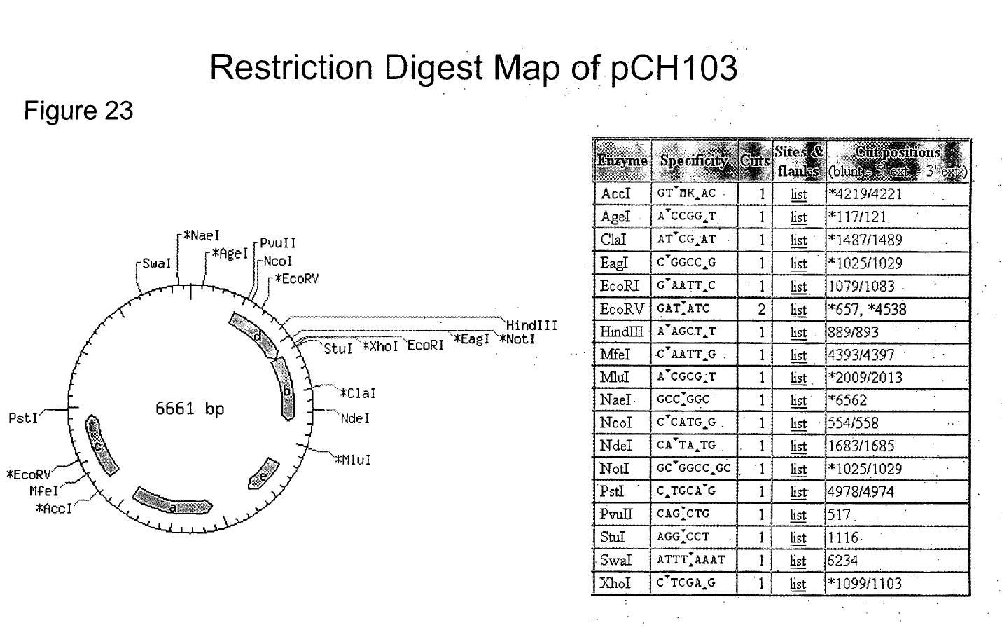

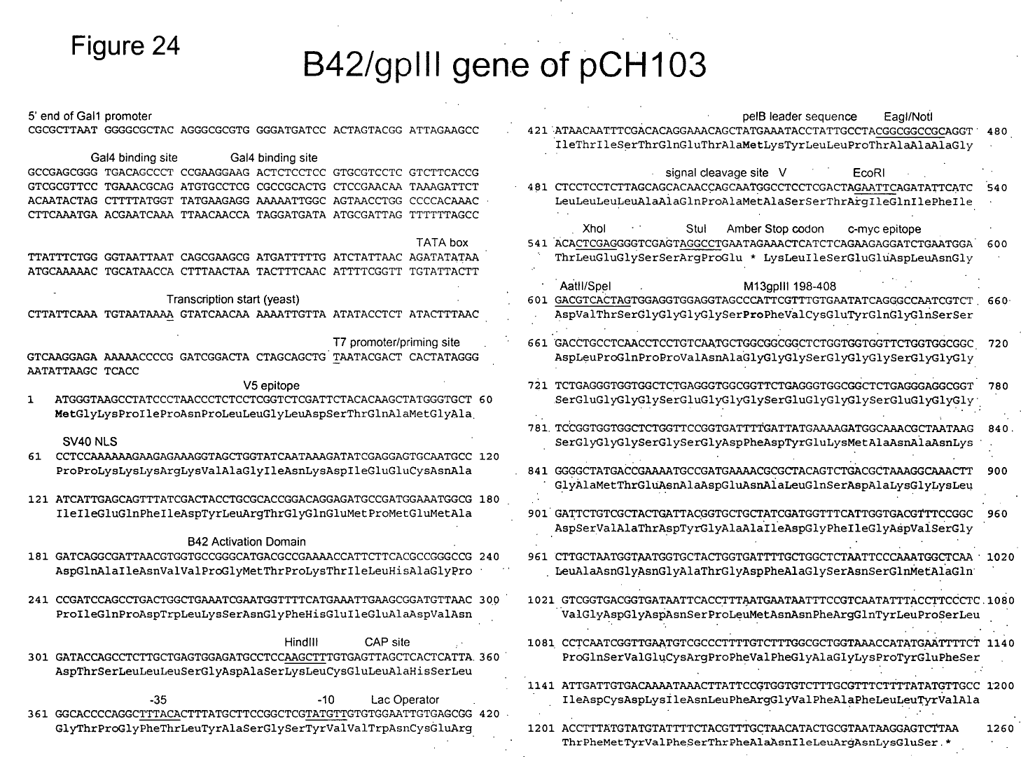

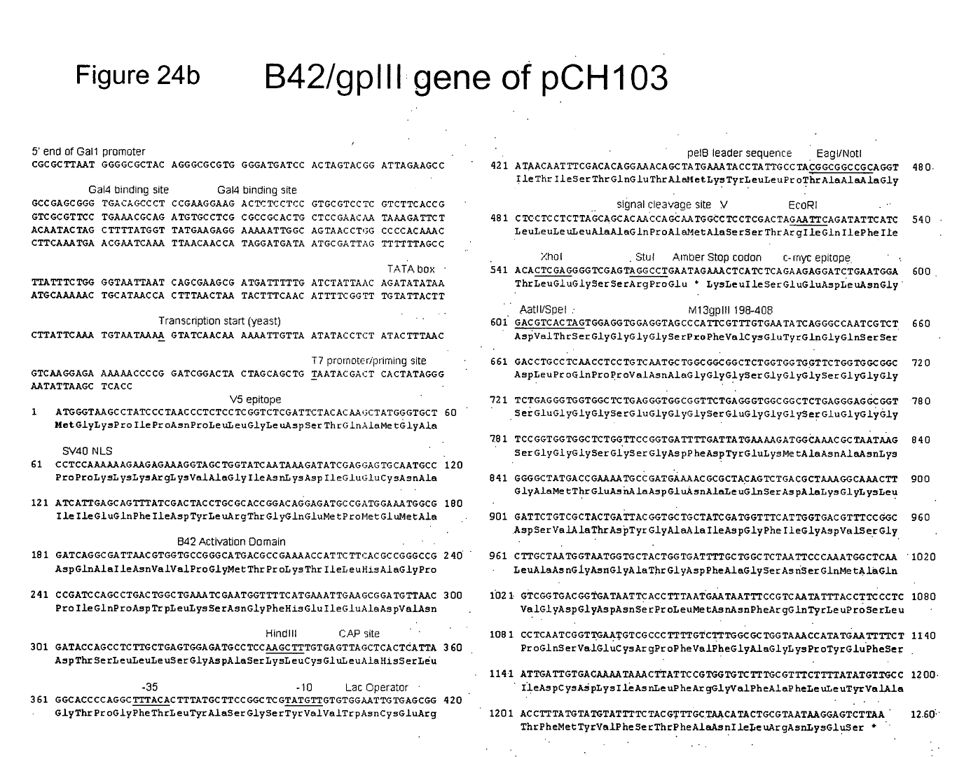

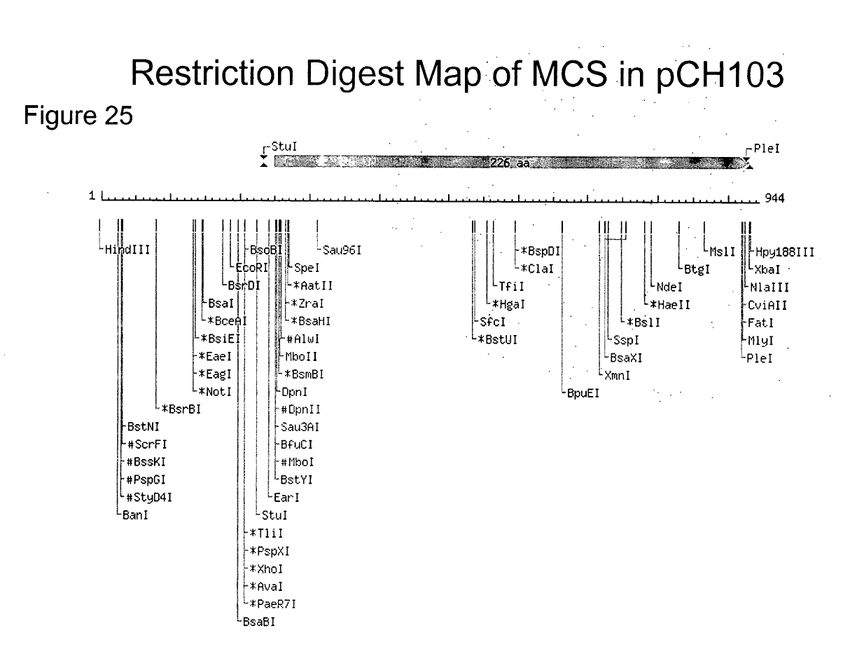

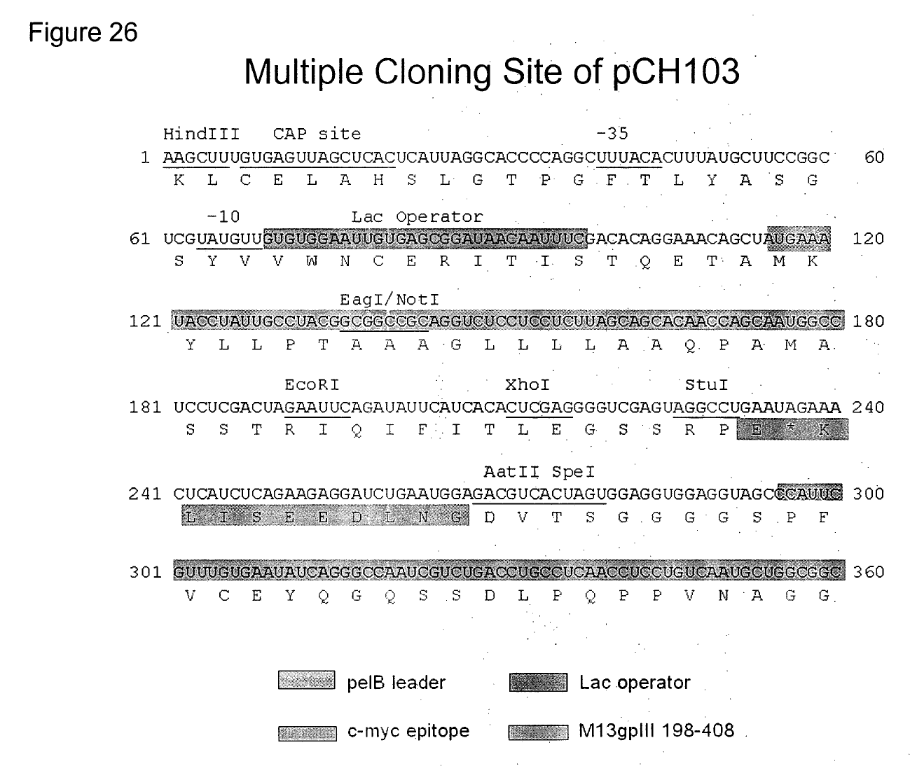

M13-based phage display is a powerful technology for expressing, selecting and/or engineering peptides and proteins expressed on the surface of a filamentous bacteriophage, such as M13 bacteriophage. Prefered fusions in M13 are to either the gpvm protein or to the gpIII protein (see FIG. 18). Large proteins are fused to the gpIII protein at the amino terminus. Alternatively, proteins are fused to the gpIII protein at the carboxy terminus, or proteins are fused to the gpVIII protein as the amino terminus or carboxy terminus. The gpVIII protein generally provides several thousand fusions per phage particle, while the gpIII protein provides about five fusions per phage particle. (See, Willats, Phage display: practicalities and prospects. Plant Mol Biol. 2002. 50:837-85.)

Phage display is also useful to create in vitro libraries from which antibodies to many different antigens are isolated. (See, Hallborn, 2002; Hoogenboom, 1998; Kay, 2001). Generally, peptides or proteins displayed on the surface of M13 need to be capable of (or modified for) secretion through the cell membrane, a necessary step in filamentous phage assembly. Methods used to create antibody diversity in phage display libraries include; i) cloning cDNA of the immune regions (native approach), of either immunized or non-immunized cells (to create a so-called naïve library), ii) total synthesis of antibody gene fragments with mixed-nucleotide synthesis, and iii) a semi-synthetic approach whereby a framework gene is synthesized, and the diversity is generated by cloning in a multitude of complementarity determining regions (CDRs, semi-synthetic approach). (See, Jirholt P, 1998; Hoogenboom 1998). Alternatively, diversity is created by replacing the V_{H} and V_{L} CDR3 regions of a set of 49 master genes by CDR3 library cassettes, generated from mixed trinucleotides and biased towards natural human antibody CDR3 sequences. (See, Knappik A, 2000).

T7 Phage Display

The T7 phage display takes advantage of the properties of bacteriophage T7. This system has the capacity to display peptides up to about 50 amino acids in size in high copy number (415 per phage), and peptides or proteins up to about 1200 amino acids in low copy number (0.1-1 per phage) or mid-copy number (5-15 per phage). Phage assembly takes place in the E. coli cytoplasm and mature phage are released by cell lysis. Peptides or proteins displayed on the surface of T7 do not need to be capable of secretion through the cell membrane, a necessary step in filamentous phage assembly. T7 is an excellent general cloning vector. Purified DNA is easy to obtain in large amounts, a high-efficiency in vitro packaging system has been demonstrated, and the DNA is completely sequenced (39,937 bp).

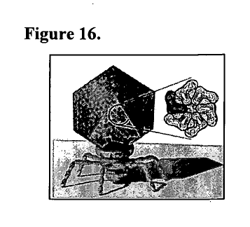

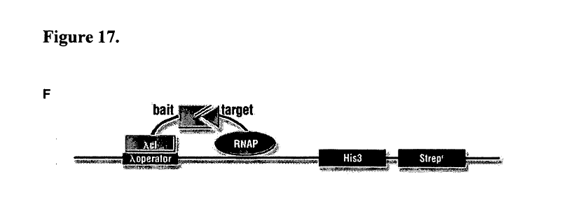

Functional proteins up to slightly more than 1000 amino acids have been displayed from T7Select1-1 vectors. Peptides or proteins are displayed in low copy number (about 0.1-1 per phage) from these vectors, which makes them suitable for the selection of proteins that bind strongly to their targets. To obtain low copy display, the promoter of the capsid gene and the translation initiation site were removed. The capsid mRNA is still produced from phage promoters located further upstream of the gene, but production of capsid protein is greatly reduced. T7Select1 phage is grown on a complementing host that provides large amounts of the 10A capsid protein from a plasmid clone. The 10A gene in the complementing plasmid and the capsid gene in the vectors have been engineered to minimize any recombination between the genes. Unlike other display systems that utilize N-terminal fusions, inserts are fused at the C-terminus of T7 gene 10, enabling the expression and display of inserts that contain internal stop codons. This is important when using oligo(dT) primed cDNA because poly(A) is usually downstream from a translation stop codon in mRNA. FIG. 16 shows the structure of the T7 phage particle. The capsid shell, head-tail connector, tail, and tail fibers are shown schematically. The diffraction pattern from polyheads (4) showing a hexamer capsid unit has been fit onto the surface of the icosahedral particle (diameter approx. 55 nm). The monomer units are in gray.

Development and Use of Specialized Phase Display Libraries

The substrate specificity of human collagenase 3 (MMP-13), a member of the matrix metalloproteinase family, was investigated using a phage-displayed random hexapeptide library containing 2.3×10⁸ independent recombinants. Phage display technology was used to identify a number of new collagenase 3 substrate sequences. Many of the new substrate sequences were similar to known cleavage sites in type II collagen or aggrecan. However, others deviated significantly from previously known cleavage sequences. These phage display results refine the understanding of the overall specificity of collagenase 3. This knowledge of collagenase 3 specificity is also useful to identify other possible substrates. Database search results support studies demonstrating that gelatinase β can degrade TGF β and provides evidence that collagenase 3 is involved in processing of the latency-associated peptide to activate this growth factor.

Arrayed cDNA Library Screening

Using a previously developed a scale invariant bead-based genotyping technology for a high throughput SNP analysis assay utilizing an array of fluorescently-differentiated microspheres (available from Luminex Corp, Austin, Tex.) covalently coupled with complementary ‘ZipCode’ DNA sequences [czipcodes; Taylor, 2001; lannone, 2000; Chen, 2000]. For the SNP assay, querying a polymorphic base involves enzymatic extension of a ‘probe’ oligonucleotide containing both the ZipCode and a SNP-specific sequence with a fluoresceinated reporter molecule (for example, a dye-labeled ddATP).

After the labeling reaction is completed, the ZipCode portion of the probe oligonucleotide hybridizes with the cZipCode on the microsphere. Each polystyrene microsphere has a different ratio of two different fluorescent dyes, allowing for discrimination of alleles from a multiplexed sample. Analysis by flow cytometry identifies both the microsphere and either the presence or absence of a flourescent signal.

This genotyping technology was adapeted to provide a means for combinatorial encoding as well as inexpensively and rapidly decoding of cDNAs in a Y2H interaction trap assay [Nelsen, 2001]. The liquid-format assay described, which was formatted in 96-well microtiter plates, was automated and scaled to accommodate large-scale, high-throughput protein interaction mapping of C. elegans (Peppers et al., manuscript submitted for publication).

In an example, (Peppers et al., submitted for publication), we used the completed genome sequence for C. elegans to create an array of proteins that were tested for interactions in a pair-wise matrix. By extensive sequence comparisons, we first selected several thousand worm genes on the basis of likely orthology to human proteins. Each of 3,293 genes were then amplified by PCR and subcloned into yeast two-hybrid bait and prey vectors. Whereas a single bait vector was used for all bait constructs, we used 88 differently “ZipCode” vectors to express the prey genes. The first 88 prey constructs, each bearing a different ZipCode, were then pooled into the first well of a 96-well microtiter plate. This was repeated for the next set of 88 genes, and so on, such that all 3,520 preys were contained in 40 wells. Thus, the combination of ZipCode and well location provided a unique identification for each prey. A duplicate set of preys were placed in another set of 40 wells for duplicate testing of protein interactions. This strategy permitted us to screen 8,448 preys in a single 96-well plate (without duplication). Higher densities could be used without loss of data (see Buckholz 1999).

Gene Assembly and Amplification, Cloning and Sequencing

The assembly of the synthetic gene from component oligonucleotides was performed according to a previously described protocol [Stemmer, 1995]. The synthetic gene, purified by gel extraction, was cloned into the vector pCR-Script [Costa, 1994] and the ligation products were transformed into DH5α E. coli cells and selected for on LB plates with 50 μg per ml ampicillin. The plasmids isolated are screened by either restriction digest analysis or PCR using primers complementary to vector sequences flanking the synthetic gene, and plasmids containing the gene of interest are sequenced in both the forward and reverse directions.

Subcloning and Testing for Antibody Activity in vitro.

The synD2E7 gene is designed to be cloned into the phage M13Sam3 [Kay, 2001; O'Connell, 2003] using SalI and AatII digestion. The SalI site is compatible with the XhoI site on the phage. The synthetic gene is cloned into the phage and resequenced to ensure that the DNA sequence is correct [Weiner, 1994]. The recombinant phage is tested for antibody activity and binding to TNFα protein. Standars methods for ELISA assays will be used Antibodies are also available to the M13 coat protein and the c-myc tags encoded on the phage particles [Terpe, 2003]. Standard bait and prey controls will be used; including i) DNA sequencing of the inserts, ii) tests for autonomous transactivation, iii) a repression assay (to test for translocation into the yeast nucleus), and iv) a Western analysis to ensure protein expression.

Epitope Cloning and Activity in Yeast

Both the full-length TNFα gene and its D2E7-associated epitope into the appropriate yeast prey vector.

Phage Display Biopanning/ELISA

A variety of solid phases have been used successfully for biopanning with phage display libraries, including plastic ELISA plates or uncoated cell culture dishes, magnetic particles, glass beads, and beaded agarose. The most convenient and commonly used solid phase is plastic and the most commonly used method for coating is non-covalent adsorption. However, because the adsorption of proteins onto plastic surfaces is thought to be a hydrophobic interaction, some ligands, particularly highly hydrophilic proteins or low molecular weight compounds, may bind inefficiently to plastic unless a covalent attachment method is used. The methods used for the preparation of ELISA plates are directly applicable to biopanning, and detailed ligand immobilization protocols can be found in enzyme immunoassay laboratory manuals. To enhance binding, proteins that adsorb poorly to plastic can be partially denatured with a chaotropic agent such as guanidine, urea, or thiocyanate, or with acid or heat. In addition, target lipids or lipoproteins can be adsorbed to plastic in the presence of deoxycholate. The solid phase used for immobilization of the target ligand usually depends on the volume of phage lysate screened. For most applications, a plastic 96-well ELISA plate (e.g., Corning, No. 25801) allows up to 10¹⁰ phage to be screened in a single well. However, when larger volumes (>0.2 ml) must be screened, uncoated 6 to 24-well plastic cell culture plates can be used. When screening very large lysate volumes (>2 ml), plastic Petri dishes can be used. Larger volumes may be required in the initial rounds of biopanning to ensure that a sufficiently representative sample has been exposed to the target ligand.

Each panning step starts with a mixture of phage, and seeks to select from that mixture phage whose displayed protein binds the target receptor. These phage are specifically “captured” by immobilizing the receptor (in our case, whole cells) on a solid surface; unbound phage are washed away, and the captured phage are eluted (still in infective form), yielding a selected subset of the original phage mixture that is called an “eluate.” Usually the eluate from the first round of selection is amplified by infecting the phage into fresh cells, and the amplified eluate then used as input to another round of selection. Altogether, two or three rounds of selection usually suffice to select for good binders-assuming, of course, the initial library contains such binders.

Target Preparation

For both biopanning and subsequent ELISA assay, aliquot 1 μg of target protein, in 300 μL phosphate buffered saline (PBS), per well in a high-binding polystyrene 96-well plate (NUNC-immuno plate with Maxisorp surface). Incubate at 4° C., overnight.

Biopanning (3-5 Rounds)
1. Aspirate target protein and wash wells 3× with 300 μL PBS with 0.05% (v/v) tween 20 (PB S-T).
2. Block wells by incubating with 300 μL of bovine serum albumin (BSA) in PBS (5 mg/mL) at RT for 1 hr.
3. Aspirate and wash wells 3× with 300 μL PBS-T.
4. For round 1: Warm PEG precipitated phage library to 37° C. Add 100 μL of diluted library precipitate (1:1 v/v in PBS-T with 5 mg/mL BSA) per well. Incubate at RT for 2 hr. For rounds 2-5: Pellet the eluate amplification in 1 mL aliquots at 13,000 rpm for 10 min. Add 100 μL of diluted supernatant (1: 1 v/v in PBS with 5 mg/mL BSA) per well. Incubate at RT for 2 hr.
5. Aspirate unbound phage, wash 3× with PBS-T.
6. Elute bound phage with 100 μL of 50 mM Glycine (pH 2.0) per well. Incubate at RT for 5 min.
7. Neutralize phage eluate by transferring it into 100 μL of 1 M TRIS (pH 8.0) in a polypropylene microfuge tube.

Eluate Amplification
1. Inoculate 1 mL 2XYT broth containing tetracycline (12.5 μg/mL), with 10 μL of overnight culture of an E. coli strain carrying the F′ episome (e.g. XL-1 Blue) and 100 μL of phage eluate.
2. Incubate at 37° C., shaking, overnight.

Eluate/Amplification Drop Titer
1. Perform serial dilutions representing 1×10⁻² mL to 1×10⁻⁹ mL aliquots of eluate in 100 μL PBS.
2. Inoculate 4 mL of LB top agar (at 55° C.) with 40 μL of overnight culture of an E. coli strain carrying the F′ episome (e.g. XL-1 Blue). Pour over LB agar plates containing tetracycline (12.5 μg/mL).
3. Using an 8-channel mulitchannel pipetter, transfer 1 μL aliquots from each serial dilution onto cooled top agar.
4. Incubate at 37° C., overnight.

Eluate Plating for Single Plaques
1. Perform serial dilutions as for drop titer.
2. Transfer aliquots representing 1×10⁻⁵ mL, 1×10⁻⁷ mL and 1×10⁻⁹ to 14 mL polypropylene tubes.
3. Inoculate 12 mL of LB top agar (at 55° C.) with 120 μL of overnight culture of an E. coli strain carrying the F′ episome (e.g. XL-1 Blue). Transfer 4 mL aliquots to each tube containing phage dilutions.
4. Mix and pour over LB agar plates containing tetracycline (12.5 μg/mL).

ELISA
1. Prepare 3 wells for each plaque to be analyzed: one with target protein, and two blank (PBS alone) as per target preparation.
2. Aspirate and wash wells 3× with 300 μL PBS-T.
3. Block wells containing target protein and one of the two blank wells by incubating with 300 μL of bovine serum albumin (BSA) in PBS (5 mg/mL) at RT for 1 hr.
4. Aspirate and wash wells 3× with 300 μL PBS-T.
5. Pellet the eluate amplification by centrifugation. Add 100 μL of supernatant to each of the three wells (target, blocked and blank). Prepare a standard curve (in duplicate) of previously titered phage with dilutions representing 1×10¹¹ to 1×10⁸ PFU/mL. This curve should be plated on an unblocked blank plate and processed in parallel with the phage amplifications to be analyzed. Incubate at RT for 2 hr.
6. Aspirate unbound phage, wash 3× with PBS-T.
7. Aliquot 100 μL per well of 1:5000 dilution of anti-M13 monoclonal antibody conjugated to horse radish peroxidase (Amersham Pharmacia, # 27-9421-01) in PBS. Incubate at RT for 1 hr.
8. Aspirate and wash wells 3× with 300 μL PBS-T.
9. Aliquot 90 μL per well of developing solution (2 mM 2,2′-azinobis (3-ethylbenthiazoline-6-sulfonic acid (ABTS), 24.3 mM citric acid, 51.4 mM dibasic sodium phosphate, 0.03% hydrogen peroxide). Incubate at RT for up to 1 hr. Reaction can be stopped by adding 30 μL of 1% sodium dodecyl sulphate (SDS)
10. Read absorbance at 405 nm.
11. Absorbance values for blank plate should be compared to the standard curve to verify that there is no more than a 2 fold difference in the amount of phage used in the assay.
12. Absorbance values for the blocked plate should be no more than 20% of the blank plate.
13. Absorbance values of 2-fold greater than background (wildtype or empty phage vs. target protein) are considered positive (provided that the previous two conditions are met.

PCR Amplification of scFv Sequences

PCR amplification of the ScFV nucleic acids were performed as follows:

| | 1 reaction | | 100 reactions | |
|---|---|---|---|---|
| ddH₂O | 7.7 | | 770 | |
| 50X master mix buffer | 0.2 | | 20 | |
| 10X optiprime # 5 | 1 | | 100 | |
| dNTPs (10 mM) | 0.2 | | 20 | |
| SAM33forw (12.5 μM) | 0.4 | | 40 | |
| SAM33rev (12.5 μM) | 0.4 | | 40 | |
| Taq (5 U/μL) | 0.1 | | 10 | |
| | 10 | μL | 1000 | μL |

Aliquot 10 μL per reaction, inoculate with plaque or 0.5 μL of phage amplification culture (prior to pelleting).
1—Denature at 95° C. for 3 min (1×)
2—Denature at 95° C. for 30 sec
3—Anneal at 55° C. for 30 sec
4—Extend at 72° C. for 1 min
5×return to step 2 (24×)
6×Extend at 72° C. for 10 min (1×)

Optimization of (Gly₄Ser)₆ Linker Sequence

If the gene sequence is correct and it is shown that the gene is being properly expressed and displayed on the surface of the phage, but is inactive or weakly binds to the antigen, then the linker length and composition is varied to derive a phage with desired binding properties. [Volkel, 2001]. Tang et al have shown that phage display can be used to optimize the linker length [Tang, 1996].

A random linker library that varies both length and amino acid composition can be used to optimize the activity of a phage displayed antibody. There are apparently methods of linking individual V_{H} and V_{L} domains, but subtle differences in sequence dramatically influence the production, stability, and recognition properties of the scFv [Tang, 1996].

Biacore (SPR)

Hits derived from a Y2H screen are decoded by hybridization to a microscope slide containing oligonucleotides complementary to one strand of each of the 258 encoding CDRs. By judicious use of CDR DNA sequences that do not cross-hybridize, a single DNA slide is used to decode any CDR combination in a particular antibody. Using chip technology in place of DNA sequencing decreases the cost of antibody analysis and increases the rate of output.

Clones derived from the yeast screen are analyzed for any enriched CDR sequences. The multifunctional vectors are grow in E. coli, to either express or purify the antibody protein for kinetic analysis using Surface Plasmon Resonance (SPR). Refolding of E. coli-expressed antibodies is performed, as necessary, to reduce antibody congregation in inclusion bodies. This refolding is performed by cleaving denatured inclusion bodies with signal peptidase to remove the M13 leader sequence and refold the resulting protein using methods known in the art.

### EXAMPLE 2

### Design of a Random Combinatorial Antibody Library

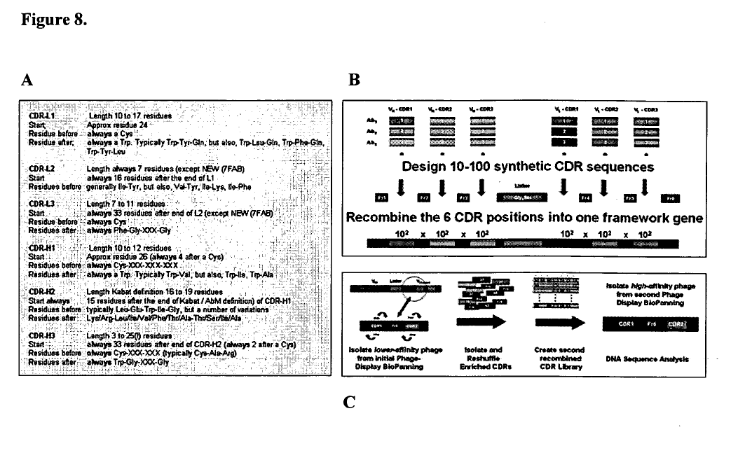

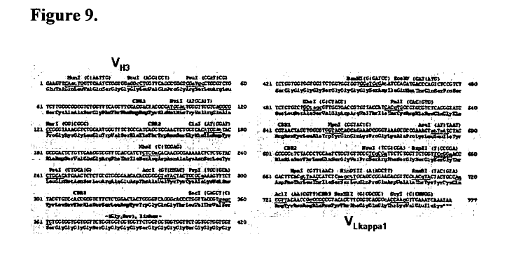

An antibody library is created using 43 different CDR sequences in each of the 3 CDRs in V_{H} and V_{L} regions of an scFv (i.e., a total of 258 different CDRs). This library contains a possible 6.3×10⁹ (i.e., 436) different antibody molecules. Several of the CDR sets are specifically chosen against antigens that will be used as controls in future tests. The CDRs are being shuffled using the CDR-shuffling scheme outlined in FIG. 8. It is estimated that this size library should have sufficient diversity to obtain at least low affinity antibodies in Y2H (with >10⁷ transformants) with K_{d}'s<10⁻⁵. As shown in FIG. 8, CDR shuffling can be used to generate Ab diversity. FIG. 8A shows the dentification of CDRs in antibody genes. FIG. 9B demonstrates that the binding of CDRs occur somewhat independent of each other, so the use of this schema creates a library of maximum diversity. The 10-100 CDRs for each of the six CDRs can be shuffled using non-palindrome restriction endonuclease enzymes to create a 10⁶-10¹² CDR library. As shown in FIG. 8C, some restriction enzymes cleave downstream from their recognition site. CDRs are chosen with at least one internal amino-acid sequence which can recode a non-palindrome [in the figure, FokI (GGATG 9/13)] site. Unique non-palindrome overhangs have been encoded at each framework-CDR junction. By using CDR sequences that occur naturally in the human repertoire we will have an increased chance of deriving non-immunogenic antibodies.

Design of Synthetic scFv Gene

The well-characterized antibodies D2E7 and B10 was used to define the framwork regions of the synthetic library. The antibody D2E7 (adalimumab, trade name Humira) that is reactive against TNFα and used to treat rheumatoid arthritis in humans was chosen for the framework of a synthetic antibody scFv gene. The D2E7 antibody is filly human.

B10, recognizes the antigen Bcr and as encoded, is able to function in a yeast-two-hybrid screen. The B10 Ab was designed using the same principles as the D2E7 and the overlap of codons and sequence between the two genes was maximized as much as possible. In the framework regions, 243 (of 250) amino acids were identical between the proteins.

The pharmacokinetic properties of D2E7 appear to be linear, with a mean apparent terminal half-life after a single intravenous dose of D2E7 ranging from 15 to 19 days [Weisman, 2003]. In addition, the D2E7 antibody contains a V_{L} from the V_{Lkappa1}, and a V_{H} from the V_{H3} subfamilies of antibodies. These subfamilies represent the most common subfamilies in humans

The D2E7 and B10 protein sequence was analyzed using the programs DNAWorks [Hoover, 2002] and GeneDesign [Weiner, 1989] DNAWorks was used to determine the best set of oligonucleotides for the synthetic gene, and GeneDesign was used to optimize the placement of the appropriate restriction enzyme sites. The synthetic gene has been designed such that appropriate restriction sites bracket each of the relevant gene features, including the V_{H}, (Gly₄Ser)₆ linker, CDRs, peptide tags and V_{L} regions. The primers were further optimized for equivalent Tₘ's, and placement at positions surrounding the CDR's for the later synthesis of germline CDR libraries. Furthermore, 8 DpnI sites were placed into the gene for potential use in selecting against parental DNA in any future mutagenesis/replacement experiments [Weiner, 1994a; Weiner, 1994b; Costa, 1994c]. In a similar way, the gene has been further designed not to include any HaeIII (5′-GGCC-3′) sites in the framework regions. The choice of codons for the synthetic gene was optimized for both yeast and E. coli.

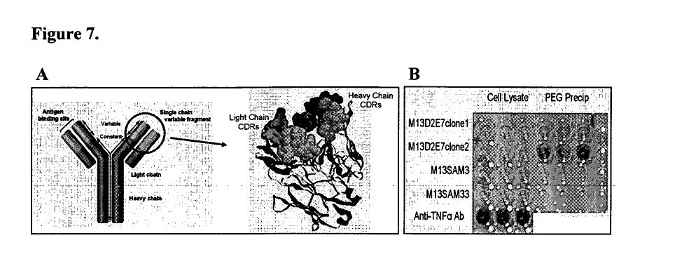

The synthetic D2E7 was genetically fused to the gpIII protein and expressed in E. coli. Phages were tested in an ELISA format (see FIG. 7, right). As shown in Figure xx, an antibody gene is fused to the M13 gpIII protein. FIG. 7B shows phage ELISA of scFv D2E7 fused to M13 SAM3. CDR shuffling (see FIG. 9) is used to generate antibody diversity of proteins genetically fused and displayed on the gpIII protein of M13. In Figure x6, the blue-highlighted sequences represent alternating sets of CDR oligonucleotide primer overlaps in the D2E7 gene. The gene encodes a 3′ c-myc and his6 encoding sequence for use in histology and purification (not shown).

Choice of CDRs

Several thousand DNA sequences exist for the V_{Lkappa1} and V_{H3} subfamilies. 10 to 100 CDR sequences were chosen from this ‘super-set’ for each of the 6 CDR sites based on several criteria, including; i) a lack of cross-hybridization of the nucleic acid sequences of the reverse-translated CDR amino acid sequence set, ii) their encoding a set of oligonucleotide set of matched Tₘs, and iii) the optimal placement of nonpalindromic (typeII) restriction endonuclease sites within the CDR encoding sequences for future subcloning purposes.

The synthetic genes were designed to encode unique, non-palindrome restriction sites at each CDR-framework junction. These sites can be restriction-digested with one or several non-palindrome recognition sequence restriction enzyme sites that are contained within either the CDR's or the framework. Several restriction enzymes have a known recognition site, but cleave the DNA several bases downstream. Examples of these enzymes include FokI, BsaI, FauI, and BfuAI. We can exploit these enzymes to create unique ends at each framework-CDR junction. Using these unique junctions, diversity in the library will be created by i) cloning synthetic fragments into a particular CDR using a pair of unique, bracketing palindrome-recognizing restriction enzymes (see figure), and/or ii) shuffling the entire gene using the non-palindrome restriction enzymes. The non-palindrome sequences are needed for this strategy because they can be designed a priori to not self-catenate when ligated. Use of several non-palindrome enzymes avoids a bias of all CDRs having the same amino acid at a particular position.

In a gene shuffling experiment, the pool of six CDRs, the framework and linker fragments will be ligated together with T4 DNA ligase, and a band of the predicted correct size will be gel isolated after 5-10 rounds of PCR amplification with two outside primers.

Design of Synthetic Genes

Publically available programs are available for the design of synthetic genes for expression in heterologous organisms programs. The programs determine nucleic acid sequences based on codon usage as specified by the user, and determine the possible placement of base changes that can be used to create restriction endonuclease sites without altering the amino acid sequence in the target protein (i.e., translational-silent mutations to the original nucleic acid sequence). The program, GeneDesign, accomplished this by reverse-translating a restriction enzyme sequence (for any given restriction enzyme) in all three reading frames to create a database of all possible peptides that could encode that restriction enzyme sequence within it. This process was reiterated for all known restriction enzymes. The program then searched the synthetic target sequence for the occurrence of any of these peptides, where a translational-silent change to the DNA sequence could then be made to encode a restriction enzyme site. This suite of programs was used to synthesize and express RNaseA and thrombin in E. coli and insect cells.

### EXAMPLE 3

### Affinity Maturation

In order to increase the binding efficiency, i.e., “affinity mature” the CDR utilized for a particular antigen were identified by using the sequences encoding a CDR as ‘ZipCodes’. CDRs that were used in the first round of antbody synthesis were then reshuffled to produce a second round of antibosies with increased affinity for an antigen.

Identification of Each of the Six CDRs

It has been previously demonstrated the method for high-throughput yeast two-hybrid analysis where the proteins of interest are encoded by known or predicted cDNA sequences [Nelson, 2002]. The method is easily automated and can be scaled to accommodate projects of varying complexity. In the example analysis of 96 clones per well, 20 ZipCodes in two “dimensions”, the dimensions containing 12 and 8 elements, respectively, were used.

A set of 100 microspheres (beads) from Luminex is used for decoding. Of these, 60 or so are robust enough for high-throughput genotyping methods (many of the other 40 beads rapidly photo-bleach under ambient light). The complement of the CDR encoding sequences is used as cZipCodes and attached to beads as previously described. After the Y2H analysis is completed, wells with β galactosidase activity greater than a defined value (generally chosen as 3× background) will be subjected to PCR using dye-labeled primers common to all clones and external to CDRs 1 and 6. By using one set of primers external to CDRs 1 and 6, only one PCR product will need to be generated with which to decode the 6 CDRs.

Decoding Assay

PCR products are hybridized against a pool of microspheres containing 250 beads per μl in 1.5 M NaCl. The pool is populated with different types of microspheres; where each type is coupled to the complement of one of the ZipCode sequences used in the CDR diversity mix. During the hybridization, samples is denatured at 96° C. for 2 minutes and incubated at 45° C. for greater than one hour. Following hybridization, samples are washed in 1×SSC containing 0.2% Tween-20, resuspended in a 1.5M NaCl solution containing streptavidin-phycoerythrin and incubated for 20 minutes at room temperature. The reactions are diluted with 60 μl of 1×SSC containing 0.2% Tween-20 prior to analysis on the LX100 (Luminex, Austin, Tex.).

Reshuffling the Positive Hits.

The pool of positive hits will be shuffled as as described above and the amplified product will be cloned by homologous recombination into a bait-containing yeast strain. The LexA DNA binding sites for the β galactosidase reporter gene will be titered so that we can use β galactosidase activity on filter lifts as an initial scoring means for antibody-antigen strength of interaction [Watson, 1999].

Data analysis. Data analysis is performed as previously described using the algorithms available from Luminex.

### EXAMPLE 4

### Megaprimer-Mediated Gene Synthesis (MPS)

Production of Megaprimers

Standard gene synthesis of a 800 bp gene requires approximately 36 oligonucleotides. To produce a library of scFvs with only 10 possible CDRs at each CDR position would require >90 oligonucleotides. Production of more useful, i.e. diverse, libraries would require use of>600 oligos. Mixing so many oligos together for reliable generation of correct, diverse synthetic genes has proven unreliable. To overcome the high rate of mutagenesis, fragments of the synthetic gene(s) were designed as megaprimers, with ≧25bp overlaps between fragments. The megaprimers are approximately 130 bp in length and serve as a series of overlapping, alternating strands of DNA encompassing the full length synthetic gene. These megaprimers were synthesized as dsDNA, with a bias to the megaprimer strand. The primers were gel purified and mixed for 25 cycles of PCR amplification to produce the full length synthetic gene. Synthesis of single genes by this method demonstrated increased fidelity over standard gene synthesis (DNAWorks). Synthesis of a library of 1×10⁶ possible sequences was successful in that diverse full length sequences were generated at a rate of 50%. Sequences occasionally did not match exactly what was put into the megaprimer mix. However, the appearance of frameshift mutations with MPS was much reduced relative to what is seen with other known methods such as DNAWorks.

Synthesis of an scFv by MPS

Megaprimers are highlighted in italic (bottom strand) and underline italic (top strand). Megaprimer primers are in bold and Complement strand primers are underlined.

F=forward primer

FH=heavy chain framework

FL=light chain framework

CDR=Complementarity Determining Region

R=reverse primer

| M1) | FFH1long mFFH2 FCDR1 4FFH3 |
|---|---|
| | 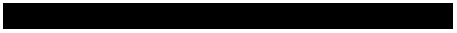 |
| M2) | 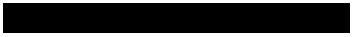 |
| | RFH3 RCDR2 4RFH4 |
| M3) | 4FFH5 FCDR3 4FL4A |
| | 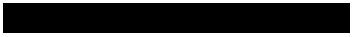 |
| LINK) | 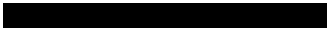 |
| 4RL4A 4RL4B 4RL4C | |
| M4) | 4FL4C FCDR4 FFL1long |
| 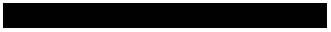 | |
| M5) | 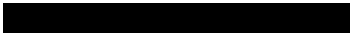 |
| RFL1 RCDR5 RFL2 | |
| M6) | 4FFL3 FCDR6 FFL4long |

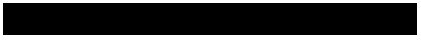

Mix oligos for each megaprimer as highlighted above.
1 uL of each stock oligo (1 ug/uL) in 69 uL sterile dH₂O

Anneal oligos (96° C. for 20 minutes, followed by slow cool down at RT)

Use each mix for Megaprimer Synthesis:
5 uL optiprime #7
1.08 uL Megaprimer primer (1 uM)

1.08 uL Complement strand primer (0.1 uM)
1.08 uL dNTPs (2 mM)
1.08 uL Taq/Pfu (16:1)
5 uL Megaprimer Annealed mix
36 uL dH₂O

Run JCWorks2: 95° C.—5 min; 95° C.—30 sec; 54° C.—30 sec; 72° C.—30 sec; repeat 9 times; 72° C.—10 min; 4° C.

Run on 2.5% Agarose gel to purify each megaprimer.

Use Qiagen gel extraction kit to purify DNA.

Megaprimer-Mediated Gene Synthesis:

ScFv genes are synthesized as follows:

Mix 7 megaprimers (6 with CDRs, 1 for linker) in a tube—5 uL each (40 ng).

Perform PCR-amplification:
5 uL optiprime #5
1.08 uL dNTPs (2 mM)
1.08 uL Taq/Pfu (16:1)

5 uL Megaprimer mix

1.08 uL FFH1 long

1.08 uL RFL4 long

36 uL dH₂O

Run CDR25PCR: 95° C.—5 min; 95° C.—30 sec; 56° C.−1 min; 72° C.—1 min; repeat 24 times; 72° C.—10 min; 4° C.

Run product on 1% agarose gel.

Use Qiagen gel extraction kit to purify DNA

Digest with restriction enzymes (EcoRI and XhoI) and clone into vector of choice (M13JW150).

Exemplary olignucleotide for designing the framework region of the sythetic antibody are shown in Table I. Exemplary olignucleotide for designing the CDRs of the sythetic antibody are shown in Table II.

TABLE 1
Framework Oligos

| | | AMINO ACIDS |
|---|---|---|
| OLIGO ID | SEQUENCE | (FRAMEWORK) |
| FFH1long | ACTGAACATGCACGTACGTCGACAGAATTCTGGAAGTTCAATTGGTTGAATCTGGTGGAG | *TCTYVDRILEVQLVESGG |
| | (SEQ ID NO:1) | (SEQ ID NO:2) |
| mFFH2 | GTCTGGTTCAGCCGGGTCGAAGCCTGCGTCTGTCTTGTGCGGCTAGC (SEQ ID NO:3) | LVQPGRSLRLSCAAS (SEQ |
| | | ID NO:4) |
| 4FFH3 | CCGGGTAAAGGTCTGGAATGGGTTTCT (SEQ ID NO:5) | PGKGLEWVS (SEQ ID |
| | | NO:6) |
| FFH3Long | TGGGTTCGTCAGGCGCCGGGTAAAGGTCTGGAATGGGTTTCT (SEQ ID NO:7) | WVRQAPGKGLEWVS (SEQ |
| | | ID NO:8) |
| 4FFH4 | CGTGACAACGCGAAAAACTCTCTGTACCTGCAGATGAACTCTCTG (SEQ ID NO:9) | RDNAKNSLYLQMNSL (SEQ |
| | | ID NO:10) |
| 4FFH5 | CGTGCGGAAGACACCGCGGTATACTACTGCGCAAAA (SEQ ID NO:11) | RAEDTAVYYCAK (SEQ ID |
| | | NO:12) |
| mRFH1 | TGACTTGTACGTGCATGCAGCTGTCTTAAGACCTT (SEQ ID NO:13) | |
| RFH2 | CAAGTTAACCAACTTAGACCACCTCCAGACCAAGTCGGCCCAGCTTCGGACGCAGAC | |
| | (SEQ ID NO:14) | |
| RFH3 | ACCCAAGCAGTCCGCGGCCCATTTCCA (SEQ ID NO:15) | |
| RFH4 | GCAAAGTGATAGAGGGCACTGTTGCGCTTTTTGAGAGACATGGAC (SEQ ID NO:16) | |
| RFH5long | AGACATGGACGTCTACTTGAGAGACGCACGCCTTCTGTGGCGCCAT (SEQ ID NO:17) | |
| 4FL4A | CTGGTTACCGTGAGCTCTGGCGGTGGTGGCTCCGGCGGTGGCGGATCTGGT (SEQ ID | LVTVSSGGGGSGGGGSG (SEQ |
| | NO:18) | ID NO:19) |
| 4FL4B | GGCGGCGGTAGCGGTGGCGGCGGATCCGATATCCAGATGACCCAGTCTCCG (SEQ ID | GGGSGGGGSDIQMTQSP |
| | NO:20) | (SEQ ID NO:21) |
| 4FL4C | TCTAGCTTAAGCGCAAGCGTTGGAGACCGAGTTACGATCACGTGC (SEQ ID NO:22) | SSLSASVGDRVTITC (SEQ |
| | | ID NO:23) |
| 4RL4A | GCCGGAGCCACCACCGCCAGAGCTCACGGTAACCAGGGTTCCTTGGCCCCA (SEQ ID | |
| | NO:24) | |
| 4RL4B | CTGGATATCGGATCCGCCGCCACCGCTACCGCCGCCACCAGATCCGCCACC (SEQ ID | |
| | NO:25) | |
| 4RL4C | TCGGTCTCCAACGCTTGCGCTTAAGCTAGACGGAGACTGGGTCAT (SEQ ID NO:26) | |
| FFL1Long | TGGTATCAGCAGAAACCCGGGAAAGCGCCGAAACTATTAATCTACC (SEQ ID NO:27) | WYQQKPGKAPKLLIY (SEQ |
| | | ID NO:28) |
| 4FFL2 | TTCTCTGGTTCTGGTTCCGGAACCGACTTCACGTTAACGAT (SEQ ID NO:29) | FSGSGSGTDFTLT (SEQ ID |
| | | NO:30) |
| 4FFL3 | CTCAAGCCTGCAACCGGAAGACGTTGCTACGTACTACTGC (SEQ ID NO:31) | LKPATGRRCYVLL (SEQ ID |
| | | NO:32) |
| FFL4long | AAGGTTGAACTCGAGGACGTCACGGCCGATGCAGGAAC (SEQ ID NO:33) | KVELEDVTADAG (SEQ ID |
| | | NO:34) |
| RFL1 | CGGCGCTTTCCCGGGTTTCTGCTGATACCA (SEQ ID NO:35) | |
| RFL2 | CCACAAGGCAGATCTAAGAGACCAAGACCAAGGCCTTGGCT (SEQ ID NO:36) | |
| RFL3long | AACGTCTTCCGGTTGCAGGCTTGAGATCGTTAACGTGAAGTCGGTTCCGG (SEQ ID | |
| | NO:37) | |
| RFL4long | GTTCCTGCATCGGCCGTGACGTCCTCGAGTTCAACCTTAGTACCCTGACCAAA (SEQ ID | |
| | NO:38) | |

TABLE II
CDR Oligos:

| | | |
|---|---|---|
| OLIGO ID | SEQUENCE | AMINO ACIDS (CDR) |

| | | |
|---|---|---|
| FCDR1AA | GGTTTCACCTTCGACGACTACGCGATGCATTGGGTTCGTCAGGCG (SEQ | GDSVSSNSAA (SEQ ID |
| | ID NO:39) | NO:40) |
| RCDR1AA | ATGCATCGCGTAGTCGTCGAAGGTGAAACCGCTAGCCGCACAAGA (SEQ | |
| | ID NO:41) | |
| FCDR1AB | GGTGATTCTGTTTCTTCTAACTCTGCTGCTTGGGTTCGTCAGGCG (SEQ | GSISSYYWS (SEQ ID |
| | ID NO:42) | NO:43) |
| RCDR1AB | AGCAGCAGAGTTAGAAGAAACAGAATCACCGCTAGCCGCACAAGA (SEQ | |
| | ID NO:44) | |
| FCDR1AC | GGTTCTATTTCTTCTTACTACTGGTCTTGGGTTCGTCAGGCG (SEQ ID | GGSVSSGSYYWS (SEQ ID |
| | NO:45) | NO:46) |
| RCDR1AC | AGACCAGTAGTAAGAAGAAATAGAACCGCTAGCCGCACAAGA (SEQ ID | |
| | NO:47) | |
| FCDR1AD | GGTGGTTCTGTTTCTTCTGGTTCTTACTACTGGTCTTGGGTTCGTCAGGCG | FTFSNYNMN (SEQ ID |
| | (SEQ ID NO:48) | NO:49) |
| RCDR1AD | AGACCAGTAGTAAGAACCAGAAGAAACAGAACCACCGCTAGCCGCACAAGA | |
| | (SEQ ID NO:50) | |
| FCDR1BC | GATGGTGGTGATGATTTCACTACTCTGTGGATTGGTTGGGTTCGTCAGGCG | GSTFSGYGVN (SEQ ID |
| | (SEQ ID NO:51) | NO:52) |
| RCDR1BC | ACCAATCCACAGAGTAGTGAAATCATCACCACCATCGCTAGCCGCACAAGA | |
| | (SEQ ID NO:53) | |
| FCDR1BD | GGTTCTACTTTCTCTGGTTACGGTGTTAACTGGGTTCGTCAGGCG (SEQ | GFTVSNYGMA (SEQ ID |
| | ID NO:54) | NO:55) |
| RCDR1BD | GTTAACACCGTAACCAGAGAAAGTAGAACCGCTAGCCGCACAAGA (SEQ | |
| | ID NO:56) | |
| FCDR2AA | CGTACTTACTACCGTTCTAAATGGTACAACGATTACGCTGTTTCTGTTCGT | RTYYRSKWYNDYAVSV |
| | TTCACTATCTCC (SEQ ID NO:57) | (SEQ ID NO:58) |
| RCDR2AA | ACCCTGGAATTTTTCAGAGTAACGAGTACCAGCGTTTTTTGGGTTAATCCA | |
| | AGAAACCCATTCCAG (SEQ ID NO:59) | |
| FCDR2AB | TACATTTACTACTCTGGTTCTACTAACTACAACCCATCTCTGCGTTTCACT | YIYYSGSTNYNPSL (SEQ |
| | ATCTCC (SEQ ID NO:60) | ID NO:61) |
| RCDR2AB | CTGAGCGTAGTTAGTACCACCAGAGTTTGGGTTAATCCAAGAAACCCATTC | |
| | CAG (SEQ ID NO:62) | |
| FCDR2AC | TACATTTACTACTCTGGTTCTACTAACTACAACCCATCTCTGCGTTTCACT | YIYYSGSTNYNPSL (SEQ |
| | ATCTCC (SEQ ID NO:63) | ID NO:64) |
| RCDR2AC | GTACAGTGGAGAGTTCCAAGCCAGGTATTTAGAGTTAATCCAAGAAACCCA | |
| | TTCCAG (SEQ ID NO:65) | |
| FCDR2AD | TGGGTTTCTACTATTACTTCTCCTAACTCTACTCACTACGCTGATTCTGTT | WVSTITSRNSTHYADSVNG |
| | AACGGTCGTTTCACTATCTCC (SEQ ID NO:66) | (SEQ ID NO:67) |
| RCDR2AD | ACCCTGCAGGTTCTGAGCGTAGTTAGTATCACCGTTGTAAACAGAAATCCA | |
| | AGAAACCCATTCCAG (SEQ ID NO:68) | |
| FCDR2BC | ATTTGGGGTGATGGTAACACTGATTACAACTCTGCTCTGCGTTTCACTATC | IWGDGNTDYNSAL (SEQ |
| | TCC (SEQ ID NO:69) | ID NO:70) |
| RCDR2BC | AACAGAATCAGCGTAGTATTTTTCAGAACCATCCTGTTTAATGTTAGAAAC | |
| | CCATTCCAG (SEQ ID NO:71) | |
| FCDR2BD | ATTTCTTACGATGGTTCTTCTACTTACTACCGTGATTCTGTTCGTTTCACT | ISYDGSSTYYRDSV (SEQ |
| | ATCTCC (SEQ ID NO:72) | ID NO:73) |
| RCDR2BD | AACAGAATCAACGTAGTATTTTTCAGAACCATCCTGTTTAATGTTAGAAAC | |
| | CCATTCCAG (SEQ ID NO:74) | |
| FCDR3AA | GATGATGATTCTAAAGAACGTAACGCTTTCGATATTTGGGGCCAAGGAACC | DDDSKERNAFDI (SEQ ID |
| | (SEQ ID NO:75) | NO:76) |
| RCDR3AA | AATATCGAAAGCGTTACGTTCTTTAGAATCATCATCTTTTGCGCAGTAGTA | |
| | (SEQ ID NO:77) | |
| FCDR3AB | GATTGCTACGGTCTGCTGGATTACTGGGGCCAAGGAACC (SEQ ID | DCYGLLDY (SEQ ID |
| | NO:78) | NO:79) |
| RCDR3AB | GTAATCCAGCAGACCGTAGCAATCTTTTGCGCAGTAGTA (SEQ ID | |
| | NO:80) | |
| FCDR3AC | GATCTGCGTTCTGGTTGGTATCCAAACTACTACTACTACGGTATGGATGTT | DLRSGWYPNYYYYGMDV |
| | TGGGGCCAAGGAACC (SEQ ID NO:81) | (SEQ ID NO:82) |
| RCDR3AC | AACATCCATACCGTAGTAGTAGTAGTTTGGATACCAACCAGAACGCAGATC | |
| | TTTTGCGCAGTAGTA (SEQ ID NO:83) | |
| FCDR3AD | ATTCCAGTTCAGCGTATGCCAACTGAAGCTTTCGATATTTGGGGCCAAGGA | IPVQRMPTEAFDI (SEQ |
| | ACC (SEQ ID NO:84) | ID NO:85) |
| RCDR3AD | AATATCGAAAGCTTCAGTTGGCATACGCTGAACTGGAATTTTTGCGCAGTA | |
| | GTA (SEQ ID NO:86) | |
| FCDR3BC | GAACGTGATTACCGTCTGGATTACTGGGGCCAAGGAACC (SEQ ID | ERDYRLDY (SEQ ID |
| | NO:87) | NO:88) |
| RCDR3BC | GTAATCCAGACGGTAATCACGTTCTTTTGCGCAGTAGTA (SEQ ID | |
| | NO:89) | |
| FCDR3BD | CCAAAAGTTCCAGGTTACAACCTGAACTGGTTCGCTTACTGGGGCCAAGGA | PKVPGYNLNWFAY (SEQ ID |
| | ACC (SEQ ID NO:90) | NO:91) |
| RCDR3BD | GTAAGCGAACCAGTTCAGGTTGTAACCTGGAACTTTTGGTTTTGCGCAGTA | |
| | GTA (SEQ ID NO:92) | |
| FCDR4AA | TCTGGTGATGCTCTGCCAAAAAAATACGCTTACTGGTATCAGCAGAAA | SGDALPKKYAY (SEQ ID |
| | (SEQ ID NO:93) | NO:94) |
| RCDR4AA | GTAAGCGTATTTTTTTGGCAGAGCATCACCAGAGCACGTGATCGTAAC | |
| | (SEQ ID NO:95) | |
| FCDR4AB | TCTGGTTCTTCTTCTAACATTGGTTCTAACACTGTAAACTGGTATCAGCAG | SGSSSNIGSNTVN (SEQ |
| | AAA (SEQ ID NO:96) | ID NO:97) |
| RCDR4AB | GTTAACAGTGTTAGAACCAATGTTAGAAGAAGAACCAGAGCACGTGATCGT | |
| | AAC (SEQ ID NO:98) | |
| FCDR4AC | GGTCTGACTTCTGGTTCTGTTTCTACTTCTTACTACCCATCTTGGTATCAG | GLTSGSVSTSYYPS (SEQ |
| | CAGAAA (SEQ ID NO:99) | ID NO:100) |
| RCDR4AC | AGATGGGTAGTAAGAAGTAGAAACAGAACCAGAAGTCAGACCGCACGTGAT | |
| | CGTAAC(SEQ ID NO:101) | |
| FCDR4AD | GGTCTGTCTTCTGGTTCTGTTTCTACTTCTTACTCTCCATCTTGGTATCAG | GLSSGSVSTSYSPS (SEQ |
| | CAGAAA (SEQ ID NO:102) | ID NO:103) |
| RCDR4AD | AGATGGAGAGTAAGAAGTAGAAACAGAACCAGAAGACAGACCGCACGTGAT | |
| | CGTAAC (SEQ ID NO:104) | |
| FCDR4AE | ACTGGTACTTCTTCTGATGTTGGTACTTACAACTACGTTTCTTGGTATCAG | TGTSSDVGTYNYVS (SEQ |
| | CAGAAA (SEQ ID NO:105) | ID NO:106) |
| RCDR4AE | AGAAACGTAGTTGTAAGTACCAACATCAGAAGAAGTACCAGTGCACGTGAT | |
| | CGTAAC (SEQ ID NO:107) | |
| FCDR4AF | ACTGGTTCTTCTTCTAACATTGGTGCTGGTTACGATGTTCACTGGTATCAG | TGSSSNIGAGYDVH (SEQ |
| | CAGAAA (SEQ ID NO:108) | ID NO:109) |
| RCDR4AF | GTGAACATCGTAACCAGCACCAATGTTAGAAGAAGAACCAGTGCACGTGAT | |
| | CGTAAC (SEQ ID NO:110) | |
| FCDR5AA | TCTACTAACACTCGTTCTTCTGGTGTTCCGTCTAGA (SEQ ID | STNTRSS (SEQ ID |
| | NO:111) | NO:112) |
| RCDR5AA | AGAAGAACGAGTGTTAGTAGAGTAGATTAATAGTTT (SEQ ID | |
| | NO:113) | |
| FCDR5AD | CGTGATCGTCCATCTGGTATTGGTGTTCCGTCTAGA (SEQ ID | RDRPSGI (SEQ ID |
| | NO:114) | NO:115) |
| RCDR5AD | AATACCAGATGGACGATCACGGTAGATTAATAGTTT (SEQ ID | |
| NO:116) | | |
| FCDR5AF | GGTTCTAAACGTCCATCTGGTGGTGTTCCGTCTAGA (SEQ ID | GSKRPSG (SEQ ID |
| | NO:117) | NO:118) |
| RCDR5AF | ACCAGATGGACGTTTAGAACCGTAGATTAATAGTTT (SEQ ID | |
| | NO:119) | |
| FCDR5AH | AACGCTTCTTCTCTGCAATCTGGTGTTCCGTCTAGA (SEQ ID | NASSLQS (SEQ ID |
| | NO:120) | NO:121) |
| RCDR5AH | AGATTGCAGAGAAGAAGCGTTGTAGATTAATAGTTT (SEQ ID | |
| | NO:122) | |
| FCDR5AJ | AAAGATTCTGAACGTGCTTCTGGTGTTCCGTCTAGA (SEQ ID | KDSERAS (SEQ ID |
| | NO:123) | NO:124) |
| RCDR5AJ | AGAAGCACGTTCAGAATCTTTGTAGATTAATAGTTT (SEQ ID | |
| | NO:125) | |
| FCDR5AM | GAAGATTCTAAACGTCCATCTGGTGTTCCGTCTAGA (SEQ ID | EDSKRPS (SEQ ID |
| | NO:126) | NO:127) |
| RCDR5AM | AGATGGACGTTTAGAATCTTCGTAGATTAATAGTTT (SEQ ID | |
| | NO:128) | |
| FCDR6AA | TACTCTACTGATTCTTCTGGTAACCCACTGTTTGGTCAGGGTACT (SEQ | YSTDSSGNPL (SEQ ID |
| | ID NO:129) | NO:130) |
| RCDR6AA | CAGTGGGTTACCAGAAGAATCAGTAGAGTAGCAGTAGTACGTAGC (SEQ | |
| | ID NO:131) | |
| FCDR6AB | TGGGATGATTCTCTGAACGGTCTGTTTGGTCAGGGTACT (SEQ ID | WDDSLNGL (SEQ ID |
| | NO:132) | NO:133) |
| RCDR6AB | CAGACCGTTCAGAGAATCATCCCAGCAGTAGTACGTAGC (SEQ ID | |
| | NO:134) | |
| FCDR6AC | GTTCTGTACATGGGTTCTGGTATTTGGGTTTTTGGTCAGGGTACT (SEQ | VLYMGSGIWV (SEQ ID |
| | ID NO:135) | NO:136) |
| RCDR6AC | AACCCAAATACCAGAACCCATGTACAGAACGCAGTAGTACGTAGC (SEQ | |
| | ID NO:137) | |
| FCDR6AE | TCTTCTTACACTTCTTCTTCTACTGGTGTTTTTGGTCAGGGTACT (SEQ | SSYTSSSTGV (SEQ ID |
| | ID NO:138) | NO:139) |
| RCDR6AE | AACACCAGTAGAAGAAGAAGTGTAAGAAGAGCAGTAGTACGTAGC (SEQ | |
| | ID NO:140) | |
| FCDR6AF | TCTTCTTACGCTGGTTCTAACAACTGGCTGTTTGGTCAGGGTACT (SEQ | SSYAGSNNWL (SEQ ID |
| | ID NO:141) | NO:142) |
| RCDR6AF | CAGCCAGTTGTTAGAACCAGCGTAAGAAGAGCAGTAGTACGTAGC (SEQ | |
| | ID NO:143) | |
| FCDR6AI | TCTTCTTACGCTGGTCGTAACTCTTTCTACGTTTTTGGTCAGGGTACT | SSYAGRNSFYV (SEQ ID |
| | (SEQ ID NO:144) | NO:145) |
| RCDR6AI | AACGTAGAAAGAGTTACGACCAGCGTAAGAAGAGCAGTAGTACGTAGC | |
| | (SEQ ID NO:146) | |

Examples of 10 CDRs not including D2E7 that were generated by MPS are shown below: The CDRs were sequenced with M13pucFor primer.

JW:10m(-)A
(SEQ ID NO:153)
TTACGATTAATTCTTAACTACTCGCCAAGGAGACAGTCATAATGAAATACCTATTGCCTACGGCGGCCGCAGGTCTCCTC
CTCTTAGCAGCACAACCAGCAATGGCCTCCTCGACTAGAATTCTGGAAGTTCAATTGGTTGAATCTGGTGGAGGTCTGGT
TCAGCCGGGTCGAAGCCTGCGTCTGTCTTGTGCGGCTAGCGATGGTGGTGATGATTTCACTACTCTGTGGATTGGTTGGG
TTCGTCAGGCGCCGGGTAAAGGTCTGGAATGGGTTTCTAACATTAAACGGGATGGTTCTGAAAAATACTACACTGATTAC
AACTCTGCTCTGCGTTTCATTATCTCCCGTGACAACGCGAAAAACTCTCTGTACCTGCAGATGAACTCTCCGCGTGCGGA
AGACACCGCGGTATACTACTGCGCAAAAGAACGTGATTACCGTCTGGATTACTGGGGCCAAGGAACCCTGGTTACCGTGA
GCTCTGGCGGTGGTGGCTCCGGCGGTGGCGGATCTGGTGGCGGCGGTAGCGGTGGCGGCGGATCCGATATCCAGATGACC
CAGTCTCCGTCTAGCTTAAGCGCAAGCCTTGGAGACCGAGTTACGATCACGTGCTCTGATTCTTCTTCTAACATTGGTTC
TAACACTGTAAACTGGTATCAGCAGAAACCCGGGAAAGCGCCGAAACTATTAATCTACCGTGATCGTCCATCTGGTATTG
GTGTTCCGTCTAGATTCTCTGGTTCTGGTTCCGGAACCGACTTCACGTTAACGATCTCAAGCCTGCAACCGGAAGACGTT
GCTATGTACTACTGCTGGGATGATTCTCTGAACGGTCTGTTTGGTCAGGGTACTAAGGTTGAACTCGAGAGGGACGTCAC
TAGTGGAGGTGGAGGTAGCCCATTCGTTTGTGAATATCAGGGCCAATCGTCTGACCTGCCTCAACCTCCTGTCAATGCTG
GCGGCGGCTCTGGTGGTGGTTCTGGTTGCCGGCTCTGAGGGTGGTGGCTCTGA
(SEQ ID NO:154)
ILEVQLVESGGGLVQPGRSLRLSCAASDGGDDFTTLWIGWVRQAPGKGLE 50
WVSNIKRDGSEKYYTDYNSALRFIISRDNAKNSLYLQMNSPRAEDTAVYY 100
CAKERDYRLDYWGQGTLVTVSSGGGGSGGGGSGGGGSGGGGSDIQMTQSP 150
SSLSASVGDRVTITCSDSSSNIGSNTVNWYOOKPGKAPKLLIYRDRPSGI 200
GVPSRFSGSGSGTDFTLTISSLQPEDVANYYCWDDSLNGLFGQGTKVELE 250
RDVTSGGGGSPFVCEYQGQSSDLPQPPVNA
1BB/2BD/3BC/4AB/5AD/6AB
JW:10m(-)B
(SEQ ID NO:155)
TTAATTCTTAACTACTCGCCAAGGAGACAGTCATAATGAAATACCTATTGCCTACGGCGGCCGCAGGTCTCCTCCTCTTA
GCAGCACAACCAGCAATGGCCTCCTCGACTAGAATTCTGGAAGTTCAATTGGTTGAATCTGGTGGAGGTCTGGTTCAGCC
GGGTCGAAGCCTGCGTCTGTCTTGTGCGGCTAGCGGTTTCACATTCTCTTCATCAGCTTCTCATTGGGTTCGTCAGGCGC
CGGGTAAAGGTCTGGAATGGGTTTCTGCAATTTCTGGTTCTGGATCTAACACTTACTATGCTGACTCAGTCAAGGGACGT
TTCACTATCTCCCGTGACAACGCGAAAAACTCTCTGTACCTGCAGATGAACTCTCTGCGTGCGGAAGACACCGCGGTATA
CTACTGCGCAAAAGGAGACGGTTACTCTTACGGATCACCTGATTGGGGCCAAGGAACCCTGGTTACCGTGAGCTCTGGCG
GTGGTAGCTCCGGCGGTGGCGGATCTGGTGGCGGCGGTAGCGGTGGCGGCGGATCCGATATCCAGATGACCCAGTCTCCG
TCTAGCTTAAGCGCAAGCGTTGGAGACCGAGTTACGATCACGTGCAGAGCATCACAGGGAATAAGGAACGATTTGGGTCG
GTATCAGCAGAAACCCGGGAAAGCGCCGAAACTATTAATCTACGCTGCTTCAACATTACAGTCGGGTGTTCCGTCTAGAT
TCTCTGGTTCTGGTTCCGGAACCGAATTTACGTTAACGATCTCAAGCCTGCAACCGGAAGACG
(SEQ ID NO:156)
ILEVQLVESGGGLVQPGRSLRLSCAASGFTFSSSASHWVRQAPGKGLEWV 50
SAISGSGSNTYYADSVKGRFTISRDNAKNSLYLQMNSLRAEDTAVYYCAK 100
GDGYSYGSPDWGQGTLVTVSSGGGSSGGGGSGGGGSGGGGSDIQMTQSPS 150
SLSASVGDRVTITCRASQGIRNDLGRYQQKPGKAPKLLIYAASTLQSGVP 200
SRFSGSGSGTEFTLTISSLQPED
1B10/2B10/3B10/4B10/5D2E7/6?? Short sequence
JW:10m(-) C
(SEQ ID NO:157)
TTAATTCTTAACTACTCGCCAAGGAGACAGTCATAATGAAATACCTATTGCCTACGGCGGCCGCAGGTCTCCTCCTCTTA
GCAGCACAACCAGCAATGGCCTCCTCGACTAGAATTCTGGAAGTTCAATTGGTTGAATCTGGTGGAGGTCTGGTTCAGCC
GGGTCGAAGCCTGCATCTGTCTTGTGCGGCTAGCGGTTTCACTTTCTCTGGTTACGGTGTTAACTGGGTTCGTCAGGCGC
CGGGTAAAGGTCTGGAATGGGTTTCTAACATTAAACAGGATGGTACTGAAAAATACTACGTTGATTCTGTTCGTTTCACT
GTCTCCCGTGACAACGCGAAAAACTCTCTGTACCTGCAGATGAACTCTCTGCGTGCGGAAGACACCGCGGTATACTACTG
CGCAAAAGGAGACGGTTACTCTTACGGATCACCTGATTGGGGCCAAGGAACCCTGGTTACCGTGAGCTCTGGCGGTGGTG
GCTCCGGCGGTGGCGGATCTGGTGGCGGCGGTAGCGGTGGCGGCGGATCCGATATCCAGATGACCCAGTCTCCGTCTAGC
TTAAGCGCAAGCGTTGGAGACCGAGTTACGATCACGTGCAGAGCATCACAGGGAATAAGGAACGATTTGGGCTGGTATCA
GCAGAAACCCGGGAAAGCGCCGAAACTATTAATCTACAAAGATTCTGAACGTGCTTCTGGTGTTCCGTCTAGATTCTCTG
GTTCTGGTTCCGGAACCGACTTCACGTTAACGATCTCAAGCCTGCAACCGGAAGACGTTGCTACGTACTACTGCTCTTCT
TACGCTGGTTCTAACAACTGGCTGTTTGGTCAGGGTACTAAGGGTGAACTCGAGAGGGACGTCACTAGTGGAGGTGGAGG
TAGCCCATTCGTTTGTGAATATCAGGGCCAATCGTCTGACCTGCCTCAACCTCCTTGTCAATGCTGGCGGCGGCTCTGGT
GGTGGTTCTGGTGGCGGC
(SEQ ID NO:158)
ILEVQLVESGGGLVQPGRSLHLSCAASGFTFSGYGVNWVRQAPGKGLEWV 50
SNIKQDGTEKYYVDSVRFTVSRDNAKNSLYLQMNSLRAEDTAVYYCAKGD 100
GYSYGSPDWGQGTLVTVSSGGGGSGGGGSGGGGSGGGGSDIQMTQSPSSL 150
SASVGDRVTITCRASQGIRNDLGWYQQKPGKAPKLLIYKDSERASGVPSR 200
FSGSGSGTDFTLTISSLQPEDVATYYCSSYAGSNNWLFGQGTKGELERDV 250
TSGGGGSPFVCEYQGQSSDLPQPPCQCWRRLWWWFWWR
1??/2BD/3??/4thrompospondin/5AJ/6AF
JW:10m(-)D
(SEQ ID NO:159)
GAATTAATTCTTAACTACTCGCCAAGGAGACAGTCATAATGAAATACCTATTGCCTACGGCGGCCGCAGGTCTCCTCCTC
TTAGCAGCACAACCAGCAATGGCCTCCTCGACTAGAATTCTGGAAGTTCAATTGGTTGAATCTGGTGGAGGTCTGGTTCA
GCCGGGTCGAAGCCTGCGTCTGTCTTGTGCGGCTAGCGGTTATACTTTTACTTCTTATGCTATGCATTGGGTTCGTCAGG
CGCCGGGTAAAGGTCTGGAATGGGTTTCTTGGATTAATGCTGGTAATGGTAATACTAAATATTCTCAAAAATTTCAAGGT
CGTTTCACTATCTCCCGTGACAACGCGAAAAACTCTCTGTACCTGCAGATGAACTCTCTGCGTGCGGAAGACACCGCGGT
ATACTACTGCGCAAAATTGACTAGAAATAAATTTAAATCTAGAGGTCATTGGGGCCAAGGAACCCTGGTTACCGTGAGCT
CTGGCGGTGGTGGATCTGGTGGCGGCGGTAGCGGTGGCGGCGGATCCGATATCCAGATGACCCAGTCTCCGTCTAGCTTA
AGCGCAAGCGTTGGAGACCGAGTTACGATCACGTGCCAAGGTGATTCTTTGAGATCTTATTATGCTTCTTGGTATCAGCA
GAAACCCGGGAAAGCGCCGAAACTATTAATCTACGGTAAAAATAATAGACCATCTGGTGTTCCGTCTAGATTCTCTGGTT
CTGGTTCCGGAACCGACTTCACGTTAACGATCTCAAGCCTGCAACCGGAAGACGTTGCTACGTACTACTGCAATTCTAGA
GATTCTTCTGGTAATCATGTTGTTTTTGGTCAGGGTACTAAGGTTGAACTCGAGAGGGACGTCACTAGTGGAGGTGGAGG
TAGCCCATTCGTTTTGTGAATATCAGGGTCCATCGTCTGACCTGCCTCAACCTCCTGTCATGCTGGCGGCGGCTCTGGTG
GTGGTTTCTGGTGGCCGGCTCTGA
(SEQ ID NO:160)
ILEVQLVESGGGLVQPGRSLRLSCAASGYTFTSYAMHWVRQAPGKGLEWV 50
SWINAGNGNTKYSQKFQGRFTISRDNAKNSLYLQMNSLRAEDTAVYYCAK 100
LTRNKFKSRGHWGQGTLVTVSSGGGGSGGGGSGGGGSDIQMTQSPSSLSA 150
SVGDRVTITCQGDSLRSYYASWYQQKPGKAPKLLIYGKNNRPSGVPSRFS 200
GSGSGTDFTLTISSLQPEDVATYYCNSRDSSGNHVVFGQGTKVELERDVT 250
SGGGGSPFVL
1-C/2-C/3AF/4AI/5AI/6AU
JW:10m(-)E
(SEQ ID NO:161)
AATTAATTCTTAACTACTCGCCAAGGAGACAGTCATAATGAAATACCTATTGCCTACGGCGGCCGCAGGTCTCCTCCTCT
TAGCAGCACAACCAGCAATGGCCTCCTCGACTAGAATTCTGGAAGTTCAATTGGTTGAATCTGGTGGAGGTCTGGTTCAG
CCGGGTCGAAGCCTGCGTCTCTCTTGTGCGGCTAGCGGTTTCACATTCTCTTCATCAGCTTCTCATTGGGTTCGTCAGGC
GCCGGGTAAAGGTCTGGAATGGGTTTCTGCAATTTCTGGTTCTGGATCTAACACTTACTATGCTGACTCAGTCAAGGGAC
GTTTCACTATCTCCCGTGACAACGTGAAAAACTCTCTGTACCTGCAGATGAACTCTCTGCGTGCGGAAGACACCGCGGTA
TACTACTGCGCAAAAGGAGACGGTTACTCTTACGGATCACCTGATCGGGGCCAAGGAACCCTGGTTACCGTGAGCTCTGG
CGGTGGTGGCTCCGGCGGTGGCGGATCTGGTGGCGGCGGTAGCGGTGGCGGCGGATCTGATATCCAGATGACCCAGTCTC
CGTCTAGCTTAAGCGCAAGCGTTGGAGACCGAGTTACGATCACGTGCTCTGGTGATGCTCTGCCAAAAAAATACGCTTAC
TGGTATCAGCAGAAACCCGGGAAACCGCCGAAACTATTAATCTACTCTACTAACACTCGTTCTTCTGGTGTTCCGTCTAG
ATTCTCTGGTTCTGGTTCCGGAACCGACTTCACGTTAACGATCTCAAGCCTGCAACCGGAAGACGTTGCTACGTACTACT
GCTACTCTACTGATTCTTCTGGTAACCCACTGTTTGGTCAGGGTACTAAGGTTGAACTCGAGAGGGACGTCACTAGTGGA
GGTGGAGGTAGCCCATTCGTTTGTGATATCAGGGCCAATCGTCTGACCTGCCTCAACCTCCTGTCAATGCTGGCGGCGGC
TCTGGTGGTGGGTC
(SEQ ID NO:162)
ILEVQLVESGGGLVQPGRSLRLSCAASGFTFSSSASHWVRQAPGKGLEWV 50
SAISGSGSNTYYADSVKGRFTISRDNVKNSLYLQMNSLRAEDTAVYYCAK 100
GDGYSYGSPDRGQGTLVTVSSGGGGSGGGGSGGGGSGGGGSDIQMTQSPS 150
SLSASVGDRVTITCSGDALPKKYAYWYQQKPGKAPKLLIYSTNTRSSGVP 200
SRFSGSGSGTDFTLTISSLQPEDVATYYCYSTDSSGNPLFGQGTKVELER 250
DVTSGGGGSPFVCDIRANRLTCLNLLSMLAAALVVG
1B10/2B10/3B10/4AA/5AA/6AA
JW:10m(-)F
(SEQ ID NO:163)
AATTAATTCTTAACTACTCGCCAGGAGACAGTCATAATGAAATACCTATTGCCTACGGCGGCCGCAGGTCTCCTCCTCTT
AGCAGCACAACCAGCAATGGCCTCCTCGACTAGAATTCTGGAAGTTCAATTGGTTGAATCTGGTGGAGGTCTGGTTCAGC
CGGGTCGAAGCCTCCGTCTGTCTTGTGCGGCTAGCGGTTCTACTTTCTCTGGTTACGGTGTTAATTGGGTTCGTCAGGCG
CCGGGTAAAGGTCTGGAATGGGTTTCTAACATTAAACAGGATGGTTCTGAAAAGTACTACGTTGATTCTGTTCGTTTCAC
TATCTCACGTGACAACGCGAAAAACTCTCTGTACCTGCAGATGATCTCTGCGTGCGGAACACACCGCGGTATACTACTGC
GCAAAACCAAAAGTTCCAGGTTACAACCTGGACTGGTTCGCTTACTGGGGCCAAGGAACCCTGGTTACTGTGAGCTCTGA
CGGTGGTGGCTCCGGCGGTGGCGGCGGATCCGATATCCAGATGACCCAGTCTCCGTCTAGCTTAAGCGCAAGCGTTGGAG
ACCGAGTTACGATCACGTGCACTGGTTCTTCTTCTAACATTGGTGCTGGTTACGATGTTCATTGGTATCAGCAGAAACCC
GGGAAAGCGCCGAAACTATTAATCTACGAAGATTCTAAACGTCCATCTGGTGTTCCGTCTAGATTCTCTGGTTCTGGTTC
CGGAACCGACTTCACGTTAACGAACTCAAGCCTGCAACCGGAAGACGTTGCTACGTACTACTGTTCTTCTTACGCTGGTC
GTAACTCTTTCTACGTTTTTGGTCAGGGTACTAATGTTGAACTCGAGAGGGACGTCACTAGTGGGAGGTGGAAGGTAGCC
CATTCGTTTTGTGATATCAAGGGCCAATCGTCTGACCTGGCCTCAACCTCCTGTCAATGGTTGGCGCGGTCTTGTGTTGG
TTCTGGTGCCGG
(SEQ ID NO:164)
ILEVQLVESGGGLVQPGRSLRLSCAASGSTFSGYGVNWVRQAPGKGLEWV 50
SNIKQDGSEKYYVDSVRFTISRDNAKNSLYLQMISACGRHRGILLRKTKS 100
SRLQPGLVRLLGPRNPGYCEL*RWWLRRWRRIRYPDDPVSV*LKRKRWRP 150
SYDHVHWFFF*HWCWLRCSLVSAETRESAETINLRRF*TSIWCSV*ILWF 200
WFRNRLHVNELKPATGRRCYVLLFFLRWS*LFLRFWSGY*C*TREGRH*W 250
EVEGSPFVL*YQGPIV*PGLNLLSMVGAVLCW
JW:10m(-)G
(SEQ ID NO:165)
ATTACGATTATTCTTAACTACTCGCCAAGGAGACAGTCATAATGAAATACCTATTGCCTACGGCGGCCGCAGGTCTCCTC
CTCTTAGCAGCACAACCAGCAATGGCCTCCTCGACTAGAATTCTGGAAGTTCAATTGGTTGAATCTGGTGGAGGTCTGGT
TCAGCCGGGTCGAAGCCTGCGTCTGTCTTGTGCGGCTAGCGGTTTCACCTTCTCCTCATCAGCTTCTCATTGGGTTCGTC
AGGCGCCGGGTAAAGGTCTGGAATGGGTTTCTGCAATTTCTGGTTCTGGATCTAACACTTACTATGCTGACTCAGTCAAG
GGACGTTTCACTATCTCCCGTGACAACGCGAAAAACTCTCTGTACCTGCAGATGAACTCTCTCCGTGCGGAAGACACCGC
GGTATACTACTGCGCAAAAGGAGACCGGTTACTCTTCACCTGATTGGGGCCAAGGAACCCTGGTTACCGTGAGCTCTGGC
GGTGGTGGCTCCGGCGGTGGCGGATCTGGTGGCGGCGGTAGCAGTGGCGCCGGATCCGATATCCAGATGACCCAGTCTCC
GTCTAGCTTAAGCCCAAGCGTTGGAGACCGAGTTACGATCACGTGCAGAGCATCACAGGGAATAAGGAACGATTTGGGTT
GGTATCAGCAGAAACCCGGGAAAGCGCCGAAACTATTAATCTACGCTGCTTCAACATTACAGTCGAGTGTTCCGTCTAGA
TTCTCTGGTTCTGGTTCCGGAACCGACTTCACGTTAACGATCTCAGGCCTGCAACCGGAAGACGTTGCTACGTACTACTG
CCAGAAATTAAACTCTTACCCATAACTTTTGGTCAGGGTACTAAGGTTGAACTCGAGAGGGACGTCACTAGTGGAGGTGG
AGGTAGCCCATTCGTTTGTGAATATCAGGCCAATCGTCTGACCTGCCTCAACTCCTGTCAATGCTGGCGGCGGCTCTGGT
GGTGTTCTGGTGGCGGCTCTGAAGGGTGGTGGCTC
(SEQ ID NO:166)
ILEVQLVESGGGLVQPGRSLRLSCAASGFTFSSSASHWVRQAPGKGLEWV 50
SAISGSGSNTYYADSVKGRFTISRDNAKNSLYLQMNSLRAEDTAVYYCAK 100
GDRLLFT*LGPRNPGYRELWRWWLRRWRIWWRR*QWRRIRYPDDPVSV*L 150
KPKRWRPSYDHVQSITONKERFGLVSAETRESAETINLRCFNITVECSV* 200
ILWFWFRNRLHVNDLRPATGRRCYVLLPEIKLLPITFGQGTKVELERDVT 250
SGGGGSPFVCEYQANRLTCLNSCQCWRRLWWCSGGGSEGWWL
JW:10m(-)H
(SEQ ID NO:167)
ATTACGATTATTCTTAACTACTCGCCAAGGAGACAGTCATAATGAAATACCTATTGCCTACGGCGGCCGCAGGTCTCCTC
CTCTTAGCAGCACAACCAGCAATGGCCTCCTCGACTAGAATTCTGGAAGTTCAATTGGTTGAATCTGGTGGAGGTCTGGT
TCAGCCGGGTCGAAGCCTGCGTCTGTCTTGTGCGGCTAGCGGTTTCACCTTCTCCTCATCAGCTTCTCATTGGGTTCGTC
AGGCGCCGGGTAAAGGTCTGGAATGGGTTTCTGCAATTTCTGGTTCTGGATCTAACACTTACTATGCTGACTCAGTCAAG
GGACGTTTCACTATCTCCCGTGACAACGCGAAAAACTCTCTGTACCTGCAGATGAACTCTCTCCGTGCGGAAGACACCGC
GGTATACTACTGCGCAAAAGGAGACCGGTTACTCTTCACCTGATTGGGGCCAAGGAACCCTGGTTACCGTGAGCTCTGGC
GGTGGTGGCTCCGGCGGTGGCGGATCTGGTGGCGGCGGTAGCAGTGGCGGCGGATCCGATATCCAGATGACCCAGTCTCC
GTCTAGCTTAAGCCCAAGCGTTGGAGACCGAGTTACGATCACGTGCAGAGCATCACAGGGAATAAGGAACGATTTGGGTT
GGTATCAGCAGAAACCCGGGAAAGCGCCGAAACTATTAATCTACGCTGCTTCAACATTACAGTCGAGTGTTCCGTCTAGA
TTCTCTGGTTCTGGTTCCGGAACCGACTTCACGTTAACGATCTCAGGCCTGCAACCGGAAGACGTTGCTACGTACTACTG
CCACAAATTAAACTCTTACCCATAACTTTTGGTCAGGGTACTAAGGTTGAACTCGAGAGGGACGTCACTAGTGGAGGTGG
AGGTAGCCCATTCGTTTGTGAATATCAGGCCAATCGTCTGACCTGCCTCAACTCCTGTCAATGCTGGCGGCGGCTCTGGT
GGTGTTCTGGTGGCGGCTCTGAAGGGTGGTGGCTC
(SEQ ID NO:168)
ILEVQLVESGGGLVQPGRSLRLSCAASGFTFSSSASHWVRQAPGKGLEWV 50
SAISGSGSNTYYADSVKGRFTISRDNAKNSLYLQMNSLRAEDTAVYYCAK 100
GDRLLFT*LGPRNPGYRELWRWWLRRWRIWWRR*QWRRIRYPDDPVSV*L 150
KPKRWRPSYDIIVQSITGNKERFGLVSAETRESAETINLRCFNITVECSV 200
ILWFWFRNRLMVNDLRPATGRRCYVLLPEIKLLPITFGQGTKVELERDVT 250
SGGGGSPFVCEYQANRLTCLNSCQCWRRLWWCSGGGSEGWWL
JW10m(-)1 8.3
M13-puc-For
(SEQ ID NO:169)
TGATTACGAATTAATTCTTAACTACTCGCCAAGGAGACAGTCATAATGAAATACCTATTGCCTACGGC
GGCCGCAGGTCTCCTCCTCTTAGCAGCACAACCAGCAATGGCCTCCTCGACTAGAATTCTGGAAGTTC
AATTGGTTGAATCTGGTGGAGGTCTGGTTCAGCCGGGTCGAAGCCTGCGTCTGTCTTGTGCGGCTAGC
GGTTCTGCTTTCTCTGGTTACGGTGTTAACTGGGTTCGTCAGGCGCCGGGTAAAGGTCTGGAATGGGT
TTCTAACATTAAACAGGATGGTTCTGAAAAATACTACGTTGATTCTGTTCGTTTCACTATCTCCCGTG
ACAACGCGAAAAACTCTCTGTACCTGCAGATGAACTCTCTCCGTGCGGAAGACACCGCGGTATACTAC
TGCGCAAAACCAAAAGTTCCAGGTTACAACCTGAACTGGTTCGCTTACTGGGGCCAAGGAACCCTGGT
TACCGTGAGCTCTGGCGGTGGTGGCTCCGGCGGTGGCGGATCTGGTGGTGGCGGTAGCGGTGGCGGCG
GATCCGATATCCAGATGACCCAGTCTCCGTCTAGCTTAAGCGCAAGCGTTGGAGACCGAGTTACGATC
ACGTGCACTGGTTCTTCTTCTAACATTGGTGCTGGTTACGATGTTCACTGGTATCAGCAGAAACCCGG
GAAAGCGCCGAAACTATTAATCTACGAAGATTCTAAACGTCCATCTGGTGTTCCGTCTAGATTCTCTG
GTTCTGGTTCCGGAACCGACTTTCACGTTAACGATCTCAAGCCCTGCAACCGGAAGACGTTGCTACGT
ACTACTGCTCTTTCTTACGCTGGTCGTAACTCTTTCTACGTTTTTGGTCAGGGTACTAAAGGTTGAAC
TCGAGAGGGACGTCACTAGTGGAGGTGGCAGGTAGCCCATTCGTTTGTGAATATCAGGCCAATCGTCT
GACCTGCCTCACCTCTTGTCATGCTGGCGGCGGCTCTGGTGGGGTTCTGTTGCCGCTTCTGAGGTGGG
GGCCTCTTAAGGTG
aa
(SEQ ID NO:170)
EVQLVESGGGLVQPGRSLRLSCAASGSAFSGYGVNWVRQAPGKGLEWVSN
IKQDGSEKYYVDSVRFTISRDNAKNSLYLQMNSLRAEDTAVYYCAKPKVP
GYNLNWFAYWGQGTLVTVSSGGGGSGGGGSGGGGSGGGGSDIQMTQSPSS
LSASVGDRVTITCTGSSSNIGAGYDVHWYQQKPGKAPKLLIYEDSKRPSG
VPSRFSGSGSGTDFHVNDLKPCNRKTLLRTTALSYAGRNSFYVFGQGTKG
*TR
1BCmutated/2AX/3BD/4AF/5AM/6??
JW10m(-)1 8.3
M13-puc-For
(SEQ ID NO:171)
TGATTACGAATTAATTCTTAACTACTCGCCAAGGAGACAGTCATAATGAAATACCTATTGCCTACGGC
GGCCGCAGGTCTCCTCCTCTTAGCAGCACAACCAGCAATGGCCTCCTCGACTAGAATTCTGGAAGTTC
AATTGGTTGAATCTGGTGGAGGTCTGGTTCAGCCGGGTCGAAGCCTGCGTCTGTCTTGTGCGGCTAGC
GGTTTCACATTCTCTTCATCAGTTTCTCATTGGGTTCGTCAGGCGCCGGGTAAAGGTCTGGAATGGGT
TTCTGCAATTTCTGGTTCTGGATCTAACACTTGCTATGCTGACTCAGTCAAGGGACGTTTCACTATCT
CTCGTGACAACGCGAAAAACTCTCTGTACCTGCAGATGAACTCTCTGCGTGCGGAAGACACCGCGGTA
TACTACTGCGCAAAGGAGACGGTTACTCTTACGGATCACCTGATTGGGGCCAAGGAACCCTGGTTACC
GTGAGCTCTGGCGGTGGTAGCTCCGGCGGTGGCGGATCTGGTGGCGGCGGTAGCGGTGGCGGCGGATC
CGATATCCAGATGACCCAGTCTCCGTCTAGCTTAAGCGCAAGCGTTGGAGACCGAGTTACGATCACGT
GCAGAGCATCACAGGGAATAAGGAACGATTTGGGTTGGTATCAGCAGAAACCCGGGGAAGCGCCGAAA
CTATTAATCTACGCTGCTTCAACATTACAGTCGGGTGTTCCGTCTAGATTCTCTGGTTCTGGTTCCGG
AACCGACTTCACGTTAACGATCTCAAGCCTGCAACCGGAAGACGTTGCTACGTACTACTGCCAGTAAT
TAACTCTTACCCATTAACTTTTGGTCAGGGTACTAAGCTTGAACTCGAGAGGGACGTCACTAGTGGAG
GTGGAGGTAGCCCATTCGTTTGTGAATATCAGGGCCAATCGTCTGACCTGCCTCAACCTCCTGTCAAT
GCTGGCGGCGGCTCTGGTGGTGGTTCTGGTGGCGGCTCTGAGGGTTGTTGGCTCTGAGGTTGCGTTTC
TGAGGTGGCTGCTCTGACGGAGGCGGTCCGGAGGTGGTTCTGGTTCCGGGATTTTGATTATGAAAGAT
GGAAACGCTAATAGGGGCTATGACCGAAATTGCCATGAAACGCGCTAGCCTGACCCGTAACGCACTTG
ATCTGTCCCTCATCGATTACGGGCTGCCTATCCGATGGGTTTCAT
aa
(SEQ ID NO:172)
EVQLVESGGGLVQPGRSLRLSCAASGFTFSSSVSHWVRQAPGKGLENVSA
ISGSGSNTCYADSVKGRFTISRDNAKNSLYLQMNSLRAEDTAVYYCAKET
VTLTDHLIGAKEPWLP*ALAVVAPAVADLVAAVAVAADPISR*PSLRLA*
AQALETELRSRAEHHRE*GTIWVGISRNPGKRRNY*STLLQHYSRVFRLD
SLVLVPEPTSR*RSQACNRKTLLRTTASN*LLPINFWSGY*G*TR
Total length = 245
JW10m(-)3 8.3
M13-puc-For
(SEQ ID NO:173)
ATTACGATTAATTCTTAACTACTCGCCAAGGAGACAGTCATAATGAAATACCTATTGCCTACGGCGG
CCGCAGGTCTCCTCCTCTTAGCAGCACAACCAGCAATGGCCTCCTCGACTAGAATTCTGGAAGTTCA
ATTGGTTGAATCTGGTGGAGGTCTGGTTCAGCCGGGTCGAAGCCTGCGTCTGTCTTGTGCGGCTAGC
AGTTTCACATTCTCTTCATCAGCTTCTCATTGGGTTCGTCAGGCGCCGAGTAAAGGTCTGGAATGGG
TTTCTGCAATTTCTGGTTCTGGATCTAACACTTACTATGCTGACTCAGTCAAGGGACGTTTCACTAT
CTCCCGTGACAACGCGAAAAACTCTCTGTACCTGCAGACGAACTCTCTGCGTGCGGAAGACATCGCG
GTATACTACTGCGCAAAAGGAGACGGTTACTCTTACGGATCACCTGATTGGGGCCAAGGAACCCTGG
TTACCGTGAGCTCTGGCGGTGGTGGCTCCGGCGGTGGCGGATCTGGTGGCGGCGGTAGCGGTGGCGG
CGGATCCGATATCCAGATGACCCAGTCTCCGTCTAGCTTAAGCGCAAGCGTTGGAGACCGAGTTACG
ATCACGTGCAGAGCATCACAGGGAATAAGGAACGATTTGGGTTGGTATCAGCAGAAACCCGGGAAAG
CGCCGAAACTATTAATCTACGCTGCTTCAACATTACAGTCGGGTGTTCCGTCTAGATTCTCTGGTTC
TGGTTCCGGAACCCACTTCACGTTAACGATCTCAAGCCTGCAACCGGAAGACGTTGCTACGTACTAC
TGCTACTCTACTGATTCTTCTGGTCGTGGTGTTTTTGGTCAGGGTACTAAGGTTGAACTCGAGAGGG
ACGTCACTAGTGGAGGTGGACGThGCCCATTCGTTTTGTGAATATCAGGGCCAATCGTCTGACCTGC
CTCAACCTCCTGTCATGCTGGCGGCGGCTCTGGTGGTGATCTGGTGGCGGCTCTGAGGGTGGTGCTC
TGAAGGTGGCGGTCCTGATGCTGGCGGATCTGAGGAGGCGGTTCCGGGGTTGCTCTGGGTCCGTGGA
TTTGATTATGTAAAGAGTGCCAACGTTAATAAGGGGCTATGACCGAAATGCCAGTGAAACGGCATAC
TGCTG
aa
(SEQ ID NO:174)
EVQLVESGGGLVQPGRSLRLSCAASSFTFSSSASHWVRQAPSKGLEWVSA
ISGSGSNTYYADSVKGRFTISRDNAKNSLYLQTNSLRAEDIAVYYCAKGD
GYSYGSPDWGQGTLVTVSSGGGGSGGGGSGGGGSGGGGSDIQMTQSPSSL
SASVGDRVTITCRASQGIRNDLGWIQQKPGKAPKLLIYAASTLQSGVPSR
FSGSGSGTDFTLTISSLQPEDVATYYCYSTDSSGRGVFGQGTKVELE
1B10/2B10/3B10/4B10/5D2E7/6BA
JW10m(-)4 8.3
M13-puc-For
(SEQ ID NO:175)
ATTAATTCTTAACTACTCGCCAAGGAGACAGTCATAATGAAATACCTATTGCCTACGGCGGCCGCAGGTCTCCT
CCTCTTAGCAGCACAACCAGCAATGGCCTCCTCGACTAGAATTCTGGAAGTTCAATTGGTTGAATCTGGTGGAG
GTCTGGTTCAGCCGGGTCGAAGCCTGCGTCTGTCTTGTGCGTCTAGCGGTTTCACCTTCGACGACTACGCGATC
CATTGGGTTCGTCAGGCGCCGGGTAAAGGTCTGGAATGGGTTTCTGCGATCACCTGGAACTCTGGTCACATCGA
TTACGCGGACTCTGTTGAAGGTCGTTTCACTATCTCCCGTGACAACGCGAAAAACTCTCTGTACCTGCAGATGA
ACTCTCTGCGTGCGGAAGACACCGCGGTATACTACTGCGCAAAAGTTTCTTACCTGTCCACGGCCTCTTCTCTG
GACTACTGCGGCCAAAGAACCCTGGTTACCGTGAGCTCTGGCGGTGGTGGCTCCGGCGGTGGTGGATCTGGTGG
CGGCGGTAGCGGTGGCGGCGGATCCGATATCCAGATGACCCAGTCTCCGTCTAGCTTAAGCGCAAGCGTTGGAG
ACCGAGTTACGATCACGTGCTCTGCTGATGCTCTGCCAAAAAAATACGCTTACTGGTATCAGCAGAAACCCGGG
AAAGCGCCGAAACTGTTAATCTACTCTACTAACACTCGTTCTTCTGGTGTTCCGTCTAGATTCTCTGGTTCTGG
TTCCGGAACCGACTTCACGTTAACGATCTCAAGCCTGCAACCGGAAGACGTTGCTACGTACTACTGCTACTCTA
CTGATTCTTCTGGTCGTGGTGTTTTTGGTCAGGGTACTAAGGTTGAAACTCGAGAAGGGACGTCCCTAGTGGAG
GTGGAGGTAGCCATTCGTTTTGTGAATATTCAGGGCCAATCGTCTGACCTGCTCAACCCTCTTGTCATGCTGGC
GGGCGGCTCTGGTGGTGGTTCTGGAT
aa
(SEQ ID NO:176)
EVQLVESGGGLVQPGRSLRLSCASSGFTFDDYANHWVRQAPGKGLEWVSA
ITWNSGHIDYADSVEGRFTISRDNAKNSLYLQMNSLRAEDTAVYYCAKVS
YLSTASSLDYWGQRTLVTVSSGGGGSGGGGSGGGGSGGGGSDIQMTQSPS
SLSASVGDRVTITCSGDALPKKYAYWYQQKPGKAPKLLIYSTNTRSSGVP
SRFSGSGSGTDFTLTISSLQPEDVATYYCYSTDSSGRGVFGQGTKVETR
1BF/2D2E7/3D2E7/4AA/5AA/6BA
JW10m(-)5 8.3
M13-puc-For
(SEQ ID NO:177)
ATTCTTAACTACTCGCCAAGGAGACAGTCATAATGAAATACCTATTGCCTACGGCGGCCGCAGGTCTCCTCCTCTT
AGCAGCACAACCAGCAATGGCCTCCTCGACTAGAATTCTGGAAGTTCAATTGCTTGAATCTGGTGGAGGTCTGGTT
CAGCCGGGTCGAAGCCTGCGTCTGTCTTGTGCGGCTAGCGGTTTTAGTATTAATGATTATTATATGCATTGGGTTC
GTCAGGCGCCGGGTAAAGGTCTGGAATGGGTTTCTAGAATTGATCCAGAAAATGGTGATGCTGATATGACTAGATC
TTCTGGTGTTCGTTTCACTATCTCTCGTGACAACGCGAAAAACTCTCTGTACCTGCAGATGAACTCTCTGCGTGCG
GAAGACACCGCGGTATACTACTGCGCAAAAGGTATGGATTATTGGGGCCAAGGAACCCTGGTTACCGTGAGCTCTG
GCGGTGGTGGCTCCGGCGGTGGCGGATCTGGTGGCGGCGGTAGCGGTGGCGGCGGATCCGATATCCAGATGACCCA
GTCTCCGTCTAGCTTAAGCGCAGCGTTGGAGACCGAGTTACGATCACGTGCAGAGCTTCTAAATCTGTTTCTACTT
CTGGTTATTCTTATATGCACTGGTATCAGCAGAAACCCGGGAAAGCGCCGAAACTATTAATCTACTTGGTTTCTAA
TTTGGAATCTGGTGTTCCGTCTAGATTCTCTGGTTCTGGTTCCGGAACCGACTTCACGTTAACGATCTCAAGCCTG
CAACCGGAAGACGTTGCTACGTACTACTGCGTTCTGTACATGGGTTCTGGTATTTGGGGTTTTTGGTCAGGGTACT
AAGGTTGAACTCGAGACGCACGTCACTAGTGGAGGTGGGAGGTAGCCCATTCGTTTGTGAAATTCAGGGCAATCGT
CTGACCTGCCTCAACCTTCTTGTCAATGCTGGCGGCGGCTCTGGTGGTGGTTCTGGTGCGGCTCTGAGGGTGGTCG
CTCTGAAGGTGTGGGTTCTAAGGTGGCGGCTCTGTGGGAGGCGGATCCGGTGGTGGCTCTGGTTCCCGGGGAATTG
ATTTATTAAAAATGGCAAACCCTTATACGGGGCTTTTTACTGAAAATGGCTAATTATATCGCCTTATCGTTCTGA
aa
(SEQ ID NO:178)
EVQLVESGGGLVQPGRSLRLSCAASGFSINDYYMEWVRQAPGKGLEWVSR
IDPENGDADMTRSSGVRFTISRDNAKNSLYLQMNSLRAEDTAVYYCAKGM
DYWGQGTLVTVSSGGGGSGGGGSGGGGSGGGGSDIQMTQSPSSLSAALET
ELRSRAELLNLFLLLVILICTGISRNPGKRRNY*STWFLIWNLVFRLDSL
VLVPEPTSRRSQACNRKTLLRTTAFCTWVLVFGVFGQGTKVELE
Total length = 245
JW10m(-)6 8.3
M13-puc-For
(SEQ ID NO:179)
AATTAATTCTTAACTACTCGCCAAGGAGACAGTCATAATGAAATACCTATTGCCTACGGCGGCCGCAGGTCTCCTC
CTCTTAGCAGCACAACCAGCAATGGCCTCCTCGACTAGAATTCTGGAAGTTCAATTGGTTGAATCTGGTGGAGGTC
TGGTTCAGCCGGGTCGAAGCCTGCGTCTGTCTTGTGCGGCTAGCGGTTTCACCTTCGACGACTACGCGATGCATTG
GGTTCGTCAGGCGCCGGGTAAAGGTCTGGAATGGGTTTCTGCGATCACCTGGAACTCTGGTCACATCGATTACGCG
GACTCTGTTGAAGGTCGTTTCACTATCTCCCGTGACAAGGCGAAAAACTCTCTGTACCTGCAGATGAACTCTCTGC
GTGCGGAAGACACCGTGGTATACTACTGCGCAAAAGTTTCTTACCTGTCCACGCCCTCTTCTCTGGACTACTGGGG
CCAAGGAACCCTGGTTACCGTGAGCTCTCGCAGTGGTGGCTCCGGCGGTGGCGGATCTGGTGGCGGCGGTAGCGGT
GGCGGCGGATCCGATATCCAGATGACCTAGTCTCCGTCTAGCTTAAACGCAAGCGTTGGAGACCGAGTTACGATCA
CGTGCAGAGCTTTAAATCTGTTTCTACTTCTGGTTATTCTTATATGCTTGGTATCAGCAGAAACCCGGGAAAGCGC
CGAAACTATIAATCTACGCGGCCTCTACCCTACAATCTGGTGTTCCGTCTAGATTCTCTGGTTCTGGTTCCGGAAC
CGACTTCACGTTAACGATCTCAAGCCTGCAACCGGAAGACGTTGCTACGTACTACTGCTGGGATGATTCTCTGAAT
GGTCTGTTTGGTCAGGCTACTAAGGTTGAACTCGAGAGGGACGTCACTAGTGGAGGTGGAGGTAGCCCATTCGTTT
GTGATTATCGGGGCCAATCGTCTGACCTGCCTCAACCTTCCTGTCAATGCTGGCGGCGGTTCTTGTGGGTGGTTTC
TGGTGGGCCGGCTCTGAAGGTAGGGGCTTCTGAAGGGTGCCGGT
aa
(SEQ ID NO:180)
EVQLVESGGGLVQPGRSLRLSCAASGFTFDDYANHWVRQAPGKGLEWVSA
ITWNSGHIDYADSVEGRFTISRDKAKNSLYLQMNSLRAEDTVVYYCAKVS
YLSTASSLDYWGQGTLVTVSSGSGGSGGGGSGGGGSGGGGSDIQMT*SPS
SLNASVGDRVTITCRALNLFLLLVILICLVSAETRESAETINLRGLYPTI
WCSV*ILWFWFRNRLHVNDLKPATGRRCYVLLLG*FSEWSVWSGY*G*TR
Total length = 250

### EXAMPLE 5

### Site-Directed Mutagenesis Using MPS

A M6 (see below) megaprimer was synthesized with Framework oligos for the D2E7 scFv (blue) and a B10-specific CDR (red). The overlap of the CDR oligos with the framework oligos had 5 nucleotide differences (highlighted in green). This fragment was generated by MPS and cloned into PCR-script-AMP and sequenced (see below). The framework oligo sequences dictated the sequence of the final product, therefore allowing use of CDRs in the context of different framework sequences.

[image]

Expect: 61 attaacttttggtggtggtactaaggttgaactcgaggacgtcacggccgatgcaggaac 120 (SEQ ID NO: 152)

### EXAMPLE 6

### Generation of Novel Y2H Vectors

The pJG4-5 activation-domain and pEG202 DNA binding-domain plasmids used in the Brent interaction trap/two-hybrid system were altered by modifying the antibiotic resistance of the reporter plasmids. Prior to the present invention, these plasmids were both ampicillin resistant, resulting in an inefficient purification of any one plasmid from a yeast strain containing multiple plasmids that constitute the complete interaction trap. Modification of these plasmids so that each plasmid expresses either kanamycin or chloramphenicol resistance, along with the parent plasmids, the interaction trap is capable of being used in yeast with three vectors that are capable of differential selection in E. coli. This allows isolation of any one plasmid by purifying all of the interaction trap plasmids from yeast simultaneously and plating E. coli transformed with the plasmids onto the appropriate antibiotic plate to select the particular plasmid of interest.

### EXAMPLE 7

### Generation of Novel Baculovirus Vectors for Phage Display

A multi-functional vector provides the ability to direct expression of polypeptides in multiple species. This type of vector is also termed a “homing” vector, in that it is capable of protein expression in three host systems commonly used for heterologous gene expression: Escherichia coli, baculovirus, and mammalian tissue culture. This vector system generally contains: (i) a phenotypic color selection for positive cloning; (ii) a multiple cloning site for both restriction enzyme and PCR cloning; (iii) an affinity tag and C-terminal fusion; (iv) antibiotic selection for both eukaryotic and prokaryotic organisms; and (v) proper splice sites and enhancers for mammalian culture expression.

A preferred method of generating recombinant baculovirus in E. coli is by a Tn7-mediated transposition event, which is a preferred method in that it allows the generation of baculovirus in a much shorter time period than that required by other techniques, such as homologous recombination transfection and plaque purification methods. Mammalian expression components (e.g., the CMV promoter and 5′ intron and enhancer regions) are located outside of the Tn7 transposition cassette (Tn7R), which results in expression through the Tn7R region. In some situations, it is preferable to mutate one or more ATG codons to decrease translation initiating upstream from the desired fMet codon, which may inhibit production of the desired gene product. The first Met codon contains a Kozak consensus sequence. A synthetic tac promoter is placed 5′ to the T7 promoter. At the ATG (fMet) start site, cloning of a nucleic acid encoding a polypeptide of interest is provided using blunt-ended PCR cloning or restriction enzyme cloning via SfiI sites.

### EXAMPLE 8

### Generation of Multi-Fuctional Expression Systems

The present invention provides a multi-functional vector useful in a yeast two-hybrid (Y2H) system and in a phage display system, and is, optionally, useful for polypeptide expression in E. coli. The present invention removes the limiations of traditional methods for expression of heterologous proteins involving the subcloning a gene of interest into DNA vectors specific for the desired expression host, which generally require either restriction digestion or PCR amplification of the gene, subcloning into an appropriate expression vector, and DNA sequence verification.

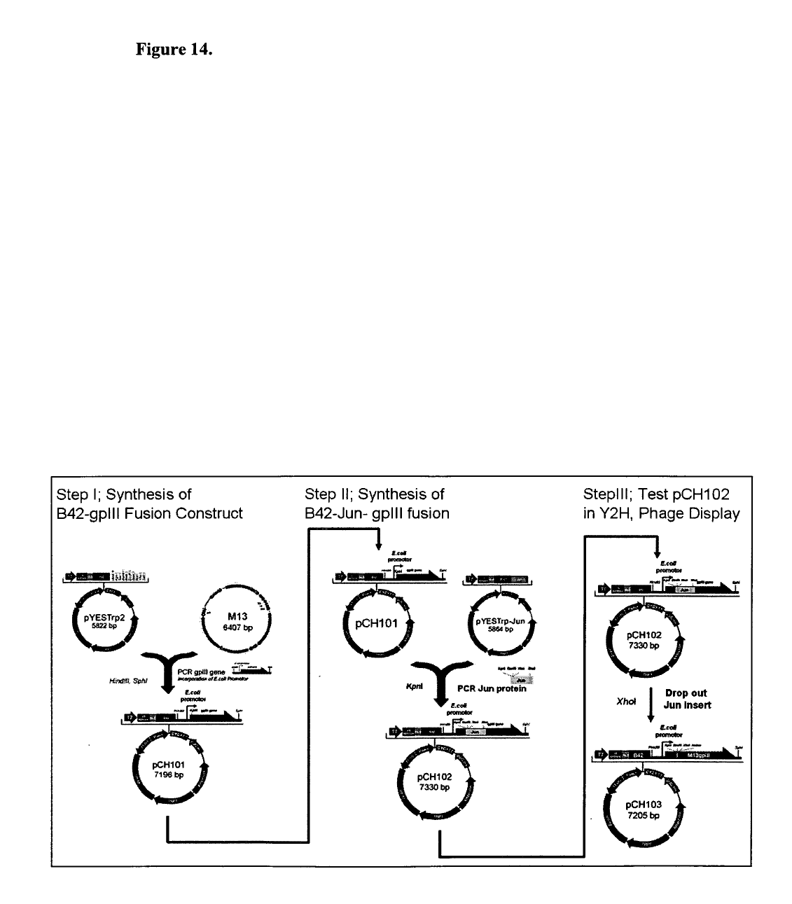

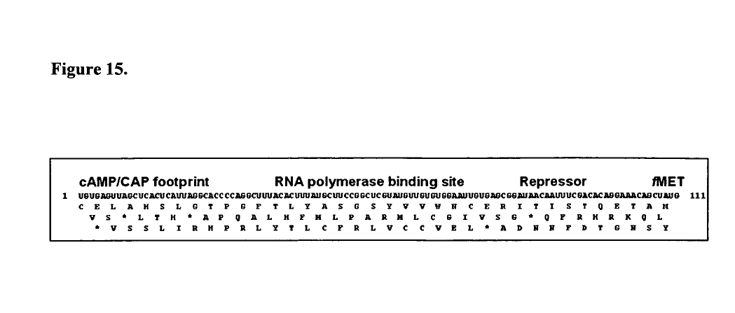

A bifunctional vector is provided herein as follows, described in three steps. In step I (FIG. 14), the gpIII gene from M13 is cloned in-frame into the pYESTrp2 vector (Invitrogen, Carlsbad, Calif.) at the HindIII/SphI site and incorporating the upstream mega-primer; 5′-aatgtgagttagctcactcattaggcaccccaggctacactatgcttccggctcgtatgttgtgtggaattgtgagcggataacaatcgacacaggaaacagctatg-3′ (SEQ ID NO: 181) and 3′ primer; 5′-acatgcgggcatgcgaactggcatgattaagactcctt-3′ (SEQ ID NO: 182). The 5′ primer is designed to allow the in-frame fusion of the gpIII protein to the B42 activation domain. In addition, it incorporates an E. coli lac promoter that encodes, in Saccharamyces cerevisiae, the amino acid sequence; ELAHSLGTPGFTLYASGSYVVWNCERITISTQETAM (SEQ ID NO: 183) (see FIGS. 14, 15). The methionine at the end of this sequence is the initiating formyl-methionine of the M13 leader peptide. The lac promoter/operator allows for regulated expression in lacI strains of E. coli. To overcome any interference of the translated lac promoter sequence peptide in the yeast two-hybrid system, the invention provides for the modification of the sequence to replace problematic amino acids.

In step II, the Jun gene that functions as a proof a principle control, from pYESTrp-Jun will be cloned into the pCH101 vector to create the pCH102 vector. The downstream primer will encode an amber (5′-UAG-3′) termination codon at the end of the Jun protein-coding sequence. This termination codon will be suppressed in strains of E. coli strains containing an appropriate amber suppressor, either supD or supE. Additional suppression can be achieved in E. coli harboring a plasmid encoding an additional suppressing gene. Such plasmids are available from Promega (Madison, Wis.). In yeast, termination will occur at this stop codon. The B42-Jun-ATS-gpIII construct in pCH102 will be tested against the Fos protein in both a yeast two-hybrid and phage display assay using standard conditions

The final cloning vector, pCH103, is created by a simple restriction enzyme cleavage and intra-molecular ligation. FIG. 15 shows the region between the yeast B42 and M13gpIII genes encodes an open reading frame of the lac promoter. In yeast this sequence will be recognized as the first reading frame peptide sequence shown above. In E. coli, this region will be recognized as a lac promoter, with the start of translation indicated as fMET.

### EXAMPEL 9

### Testing Antibody Framework in Fusion Vector System

To test the ability of an antibody to function in this system, the D2E7 and B10 antibodies are cloned into the EcoRI/XhoI sites of pCH103. The two antibodies are members of the same family, with only 7 changes in the 260 amino acids of their respective framework regions. The two antibody constructs are tested in both Y2H and phage display systems for their ability to interact with their respective antigens. Reciprocal changes are made to the two antibodies to optimize one or both as a bifunctional framework that is used in both Y2H and phage display methods.

### REFERENCES

Bach H, Mazor Y, Shaky S, Shoham-Lev A, Berdichevsky Y, Gutnick D L, Benhar I. Escherichia coli maltose-binding protein as a molecular chaperone for recombinant intracellular cytoplasmic single-chain antibodies. J Mol Biol. 2001.312:79-93.

Bird, R. E., K. D. Hardman, J. W. Jacobson, Syd Johnson, B. M. Kaufman, S. Lee, T. Lee, S. H. Pope, G. S. Riordan, M. Whitlow. Single-Chain Antigen-Binding Proteins. Science. 1988.242:423-426.

Blank K, Lindner P, Diefenbach B, Pluckthun A. Self-immobilizing recombinant antibody fragments for immunoaffinity chromatography: generic, parallel, and scalable protein purification. Protein Expr Purif. 2002. 24:313-22.

Bobrow, M., G. J. Litt, K. J. Shaughnessy, P. C. Mayer and J. Conlon. The use of catalyzed reporter deposition as a means of signal amplification in a variety of formats. Journal of Immunological Methods. 1992. 150:145-149.

Borrebaeck C A, Ohlin M. Antibody evolution beyond Nature. Nat Biotechnol. 2002. 20:1189-90

Brekke O H, Sandlie I. Therapeutic antibodies for human diseases at the dawn of the twenty-first century. Nat Rev Drug Discov. 2003.2:52-62.

Buckholz, R., C. Judelson, J. Stuart and M. Weiner. Automated format for the Yeast Two-Hybrid analysis. Journal of Molecular Microbiology and Biotechnology. 1999.1:135-140.

Casey, J. L., Coley, A. M., Tilley, L. M. and Foley, M. 2000. Green fluorescent antibodies: novel in vitro tools. Protein Engin. 13(6): 445-452.

Castillo, J., Goodson, B. and Winter, J. 2001. T7 displayed peptides as targets for selecting peptide specific scFvs from M13 scFv display libraries. J. Immunol. Meth. 257: 117-122.

Chen, J, M. A. Iannone, M. Li, J. Taylor, P. Rivers, A. J. Nelsen, K. A. Slentz-Kesler, A. Roses, and M. P. Weiner. A Microsphere-Based Assay for Single Nucleotide Polymorphism Analysis Using Single Base Chain Extension. Genome Research. 2000.10:549-557.

Choo, Y., Sánchez-Garcia, 1. and Klug, A. 1994. In vivo repression by a site-specific DNA-binding protein designed against an oncogenic sequence. Nature 372: 642-645.

Clackson, T., Hoogenboom, H. R., Griffiths, A. D. and Winter, G. 1991. Making antibody fragments using phage display libraries. Nature 352: 624-628.

Costa G L, Grafsky A, Weiner M P. Cloning and analysis of PCR-generated DNA fragments. PCR Methods Appl. 1994. 3:338-345.

Crowther D. J. Applications of microarrays in the pharmaceutical industry. Curr Opin Pharmacol. 2002. 2:551-4.

Danielsen, S., Eklund, M., Deussen, H-J., Graslund, T., Nygren, P-Å. and Borchert, T. V. 2001. In vitro selection of enzymatically active lipase variants from phage libraries using a mechanismbased inhibitor. Gene 272: 267-274.

Danner, S. and Belasco, J. G. 2001. T7 phage display: a novel genetic selection system for cloning RNA-binding proteins from cDNA libraries. Proc. Nat. Acad. Sci. USA 98(23): 12954-12959.853

De Jaeger, C., De Wilde, C., Eeckhout, D., Fiers, E. and Depicker, A. 2000. The plantibody approach: expression of antibody genes in plants to modulate plant metabolism or to obtain pathogen resistance. Plant Mol. Biol. 43: 419-428.

Debouck, C. and Goodfellow, P. N. 1999. DNA microarrays in drug discovery and development. Nature Genetics (supplement) 21: 48-50.

Dennis, M. S. and Lazarus, R. A. 1994. Kunitz domain inhibitors of tissue-factor VIIa. I. Potent inhibitors selected from libraries by phage display. J. Biol. Chem. 269: 22129-22136.

der Maur A A, Zahnd C, Fischer F, Spinelli S, Honegger A, Cambillau C, Escher D, Pluckthun A, Barberis A. Direct in vivo screening of intrabody libraries constructed on a highly stable single-chain framework. J Biol Chem. 2002. 277:45075-45085.

Donini M, Morea V, Desiderio A, Pashkoulov D, Villani M E, Tramontano A, Benvenuto E. Engineering stable cytoplasmic intrabodies with designed specificity. J Mol Biol. 2003. 330:323-332.

Dove, S L. Studying protein-protein interactions using a bacterial two-hybrid system. Methods Mol Biol. 2003. 205:251-65.

Drees, B. L. 1999. Progress and variations in two-hybrid and three hybrid technologies. Curr. Opin. Chem. Biol. 3: 64-70.

Dressman D, Yan H, Traverso G, Kinzler K W, Vogelstein B. Transforming single DNA molecules into fluorescent magnetic particles for detection and enumeration of genetic variations. Proc Natl Acad Sci USA. 2003. 100:8817-8822.

Ferrer-Miralles N, Villaverde A. Engineering of Escherichia coli beta-galactosidase for solvent display of a functional scFv antibody fragment. FEBS Lett. 2003. 533:115-118.

Gadella, T. W. J., van der Krogt, G. N. M. and Bisseling, T. 1999. GFP-based FRET microscopy in living plant cells. Trends Plant Sci. 4(7): 287-291.

Griffiths, A. D., Malmqvist, M., Marks, J. D., Bye, J. M., Embleton, M. J., McCafferty, J., Baier, M., Holliger, K. P., Gorick, B. D., Hughes-Jones, N. C., Hoogenboom, H. R. and Winter, G. 1993. Human anti-self antibodies with high specificity from phage display libraries. EMBO J. 12(2): 725-734.

Haab, B. B., Dunham, M. J. and Brown, P. O. 2001. Protein microarrays for highly parallel detection and quantification of specific proteins and antibodies in complex solutions. Genome Biol. 2(2): 1004.1-1004.13.

Hallborn J, Carlsson R. Automated screening procedure for high-throughput generation of antibody fragments. Biotechniques. 2002. Suppl:30-37.

Hamers-Casterman, C., Atarhouch, T., Muyldermans, S., Robinson, G., Memaers, C., Bajyana Songa, E., Bendahman, N. and Hamers, R. 1993. Naturally occurring antibodies devoid of light chains. Nature 363: 446-448.

Hegmans J P, Radosevic K, Voerman J S, Burgers J A, Hoogsteden H C, Prins J B. A model system for optimising the selection of membrane antigen-specific human antibodies on intact cells using phage antibody display technology. J Immunol Methods. 2002. 262:191-204.

Hoogenboom H R, de Bruine A P, Hufton S E, Hoet R M, Arends J W, Roovers R C. Antibody phage display technology and its applications. Immunotechnology. 1998. 4:1-20.

Hoogenboom, H. R. 1997. Designing and optimising library selection strategies for generating high-affinity antibodies. TIBTECH 15: 62-70.

Hoogenboom, H. R. and Winter, G. 1992. By-passing immunisation. Human antibodies from synthetic repertoires of germline VH segments rearranged in vitro. J. Mol. Biol. 227: 381-388.

Hoogenboom, H. R., de Bruïne A. P., Hufton, S. E., Hoet, R. M., Arends, J-W. and Roovers, R. C. 1998. Antibody phage display and its applications. Immunotechnology 4: 1-20.

Hoover, D. and J. Lubkowski. DNAWorks: an automated method for designing oligonucleotides for PCR-based gene synthesis. Nucleic Acids Research 2002 30:e43.

Huston J S, George A J. Engineered antibodies take center stage. Hum Antibodies. 2001. 10:127-142.

Huston, J. S.; D. Levinson; M. Mudgett-Hunter; M. Tai; J. Novotny; M. N. Margolies; R. J. Ridge; R. E. Bruccoleri; E. Haber; R. Crea; H. Oppermann. Protein Engineering of Antibody Binding Sites: Recovery of Specific Activity in an Anti-Digoxin Single-Chain Fv Analogue Produced in Escherichia coli. Proceedings of the National Academy of Sciences of the United States of America. 1988. 85:5879-5883.

Iannone, M., D. Taylor, M. Li, P. Rivers, K. Slentz-Kesler, J. Chen and M. Weiner. Mutiplexed single nucleotide polymorphism genotyping by flow cytometry. Cytometry. 2000. 39:131-140.

Irving R A, Coia G, Roberts A, Nuttall S D, Hudson P J. Ribosome display and affinity maturation: from antibodies to single V-domains and steps towards cancer therapeutics. J Immunol Methods. 2001.248:31-45.

Jirholt P, Ohlin M, Borrebaeck C A, Soderlind E. Exploiting sequence space: shuffling in vivo formed complementarity determining regions into a master framework. Gene. 1998. 215:471-476.

Johns, M., George, A. J. T. and Ritter, M. A. 2000. In vivo selection of scFv from phage display libraries. J. Immunol. Meth. 239: 137-151.

Jurado P, Ritz D, Beckwith J, de Lorenzo V, Fernandez L A. Production of functional single-chain Fv antibodies in the cytoplasm of Escherichia coli. J Mol Biol. 2002.320:1-10.

Kay, B. K. and Hoess, R. H. 1996. Principles and applications of phage display. In: B. K. Kay, J. Winter and J. McCafferty (eds.) Phage display of peptides and proteins, Academic Press, pp. 21-34.

Kay, B. K., Kasanov, J., Knight, S. and Kurakin, A. 2000. Convergent evolution with combinatorial peptides. FEBS Lett. 480: 55-62.

Kay, B K, Kasanov J, Yamabhai M. Screening phage-displayed combinatorial peptide libraries. Methods. 2001. 24:240-6.

Kirkham, P. M., Neri, D. and Winter, G. 1999. Towards the design of an antibody that recognises a given protein epitope. J. Mol. Biol. 285: 909-915.

Knappik A, Ge L, Honegger A, Pack P, Fischer M, Wellnhofer G, Hoess A, Wolle J, Pluckthun A, Virnekas B. Fully synthetic human combinatorial antibody libraries (HuCAL) based on modular consensus frameworks and CDRs randomized with trinucleotides. J Mol Biol. 2000. 296:57-86.

Kodadek, T. 2001. Protein microarrays: Prospects and problems. Chem. Biol. 8: 105-115. Lander, E. S. 1999. Array of hope. Nature Genetics (supplement) 21: 3-4.

Koide, A., S. Abbatiello, L. Rothgery, and S. Koide. Probing protein conformational changes in living cells by using designer binding proteins: Apllication to the estrogen receptor. Proceedings of the National Academy of Sciences of the United States of America. 2002. 99:1253-1258.

Labrijn A F, Koppelman M H, Verhagen J, Brouwer M C, Schuitemaker H, Hack C E, Huisman H G. Novel strategy for the selection of human recombinant Fab fragments to membrane proteins from a phage-display library. J Immunol Methods. 2002. 261:37-48.

Lowman, H. B. and Wells, J. A. 1993. Affinity maturation of human growth hormone by monovalent phage display. J. Mol. Biol. 234: 564-578.

Marks, J. D., H. R. Hoogenboom, T. P. Bonnert, J. McCafferty, A. D. Griffiths and G. Winter. By-passing immunization: human antibodies from V-gene libraries displayed on phage. J. Mol. Biol. 1991. 222:581-597.

Matthews, D. J. 1996. Substrate phage. In: B. K. Kay, J. Winter and J. McCafferty (eds.), Phage display of peptides and proteins, Academic Press, pp. 255-259.

McCafferty, J. 1996. Phage display: factors affecting panning effi-ciency. In: B. K. Kay, J. Winter and J. McCafferty (eds.), Phage display of peptides and proteins, Academic Press, pp. 261-276.

McCafferty, J. and Johnson, K. S. 1996. Construction and screening of antibody display libraries. In: B. K. Kay, J. Winter and J. McCafferty (eds.), Phage display of peptides and proteins, Academic Press, pp. 79-111.

McPherson, M. J. and Harrison, D. J. 2001. Protease inhibitors and directed evolution: enhancing plant resistance to nematodes. In: A. Berry and S. E. Radford (eds.), From protein folding to new enzymes, Cambridge University Press, Cambridge, U.K.

Mendelsohn, A. R. and Brent, R. 1999. Protein interaction methods—towards an endgame. Science 18(284): 1948-1950.

Morino, K., Katsumi, H., Akahori, Y., Iba, Y., Shinohara, M., Ukai, Y., Kohara, Y. and Kurosawa, Y. 2001. Antibody fusions with fluorescent proteins: a versatile reagent for profiling protein expression. J. Immunol. Meth. 257: 175-184.

Nelsen, A., L. Peppers, M. Rubino, D. Cyr, J. D. Kelly, J. Levy, K. Long, A. Searcy and M. Weiner. 2002. Technologies for encoding and decoding complex mixtures of arrayed clones. In Gene Cloning and Expression Technologies. Michael P. Weiner and Quinn Lu (eds). Eaton Press, Natick, Mass.

Nelsen, A., M. Weiner, L. Peppers, D. Cyr, J. D. Kelly, N. Janvier, T. Vander Ploeg, A. Ubben, A. Searcy and J. Chen. (2002). High-throughput strategies for cloning in E. coli and Yeast. In Gene Cloning and Expression Technologies. Michael P. Weiner and Quinn Lu (eds).

Nissim, A., Hoogenboom, H. R., Tomlinson, I. A., Flynn, G., Midgley, C., Lane, D. and Winter, G. 1994. Antibody fragments from a ‘single-pot’ phage display library as immunological reagents. EMBO J. 13: 692-697.

O'Connell, D., B. Becerril, A. R.-Burman, M. Daws and J. D. Marks. Phage versus Phagemid Libraries for Generation of Human Monoclonal Antibodies. Journal of Molecular Biology. 2003. 321:49-56.

Oelschlaeger P, Lange S, Schmitt J, Siemann M, Reuss M, Schmid R D. Identification of factors impeding the production of a single-chain antibody fragment in E. coli by comparing in vivo and in vitro expression. Appl Microbiol Biotechnol. 2003. 61:123-132.

Owen, M., Gandecha, A., Cockburn, B. and Whitelam, G. 1992. Synthesis of a functional anti-phytochrome single-chainFv protein in transgenic tobacco. Bio/technology 10: 790-794.

Petrenko, V. A. and Smith, G. P. 2000. Phages from landscape libraries as substitutes for antibodies. Protein Engin. 13(8): 589-592.

Plested J S, Coull P A, Gidney M A. ELISA. Methods Mol Med. 2003. 71:243-261.

Pörtner-Taliana A, Russell M, Froning K J, Budworth P R, Comiskey J D, Hoeffler J P. In vivo selection of single-chain antibodies using a yeast two-hybrid system. J Immunol Methods. 2000. 238:161-72.

Rodi, D. J. and Makowski, L. 1999. Phage-display technology—finding a needle in a vast molecular haystack. Curr. Opin. Biotechnol. 10: 87-93.

Rodi, D. J., Makowski, L. and Kay, B. K. 2001. One from column A and two from column B: the benefits of phage display in molecular-recognition studies. Curr. Opin. Chem. Biol. 6: 92-96.

Rondot, S., Koch, J., Breitling, F. and Dübel, S. 2001. A helper phage to improve single-chain antibody presentation in phage display. Nature Biotechnol. 19: 75-78.

Rottgen P, Collins J. A human pancreatic secretory trypsin inhibitor presenting a hypervariable highly constrained epitope via monovalent phagemid display. Gene. 1995. 164:243-250.

Rungpragayphan S, Kawarasaki Y, Imaeda T, Kohda K, Nakano H, Yamane T. High-throughput, cloning-independent protein library construction by combining single-molecule DNA amplification with in vitro expression. J Mol Biol. 2002. 318:395-405.

Sepp A, Tawfik D S, Griffiths A D. Microbead display by in vitro compartmentalization: selection for binding using flow cytometry. FEBS Lett. 2002. 532:455-458.

Shadidi M, Sioud M. An anti-leukemic single chain Fv antibody selected from a synthetic human phage antibody library. Biochem Biophys Res Commun. 2001. 280:548-52.

Shimada, N., Suzuki, Y., Nakajima, M., Conrad, U., Murofushi, N. and Yamaguchi, I. 1999. Expression of a functional singlechain antibody against GA24/19 in transgenic tobacco. Biosci. Biotechnol. Biochem. 63: 779-783.

Shinohara, N., Demura, T. and Fukuda, H. 2000. Isolation of a vascular cell wall-specific monoclonal antibody recognising a cell polarity by using a phage display subtraction method. Proc. Nat. Acad. Sci. USA 97(5): 2585-2590.

Sidhu, S. S. 2001. Engineering M13 for phage display. Biomol. Engin. 18: 57-63.

Slentz-Kesler, K., J. T. Moore, M. Lombard, J. Zhang, R. Hollingsworth, and M. P. Weiner. Identification of the Human Mnk2 Gene through Interaction with Estrogen Receptor Beta. Genomics 2000. 69:63-71.

Smith, G. P. 1985. Filamentous phage fusion: novel expression vectors that display cloned antigens on the surface of the viron. Science 228: 1315-1317.

Smith, M. D. and Glick, B. R. 2000. The production of antibodies in plants: an idea whose time has come? Biotechnol. Adv. 18: 85-89.

Söderlind, E., L. Strandberg, P. Jirholt, N. Kobayashi, V. Alexeiva, A. Aberg, A. Nilsson, B. Jansson, M. Ohlin, C. Wingren, L. Danielsson, R. Carlsson and C. A. K. Borrebaeck. Recombining germline-derived CDR sequences for creating diverse single-framework antibody libraries. Nature Biotechnology. 2000. 18:852-856.

Sparks, A. B., Adey, N. B., Cwirla, S. and Kay, B. K. 1996. Screening phage-displayed random peptide libraries. In: B. K. Kay, J. Winter and J. McCafferty (eds.), Phage display of peptides and proteins, Academic Press, pp. 227-253.

Steinhauer C, Wingren C, Hager A C, Borrebaeck C A. Single framework recombinant antibody fragments designed for protein chip applications. Biotechniques. (2002) Suppl:38-45.

Stemmer, W. P. C., A. Crameri, K. D. Ha, T. M. Brennan and H. L. Heyneke. Single-step assembly of a gene and entire plasmid from large numbers of oligodeoxyribonucleotides. 1995. 164:49-53.

Stockwin L H, Holmes S. The role of therapeutic antibodies in drug discovery. Biochem Soc Trans. 2003. 31:433-436.

Strauβ M., Kauder, F., Peisker, M., Sonnewald, U., Conrad, U. and Heineke, D. 2001. Expression of an abscisic acid-binding single-chain antibody influences the subcellular distribution of abscisic acid and leads to developmental changes in transgenic potato plants. Planta 213: 361-369.

Tanaka T, Chung G T, Forster A, Lobato M N, Rabbitts T H. De novo production of diverse intracellular antibody libraries. Nucleic Acids Res. 2003. 31 :e23.

Tang Y, Jiang N, Parakh C, Hilvert D. Selection of linkers for a catalytic single-chain antibody using phage display technology. J Biol Chem. 1996. 271:15682-5686.

Tawfik D S, Griffiths A D. Man-made cell-like compartments for molecular evolution. Nat Biotechnol. 1998. 16:652-656.

Taylor, D. Briley, Q. Nguyan, K. Long, M. Iannone, M. Li, E. Lai, M. Wagner, F. Ye, J. Chen and M. Weiner. A flow cytometric platform for high-throughput single nucleotide polymorphism analysis BioTechniques. 2001.30:661-669.

Terpe, K., Overview of tag protein fusions: from molecular and biochemical fundamentals to commercial systems. Appl Microbiol Biotechnol. 2003. 60:523-33. Epub 2002.

Toby G G, Golem's E A. Using the yeast interaction trap and other two-hybrid-based approaches to study protein-protein interactions. Methods. (2001). 24:201-17.

Tomlinson, I. M. and Holt, L. J. 2001. Protein profiling cones of age. Genome Biol. 2(2): 1004.1-1004.3. 854

Truong, K. and Ikura, M. 2001. The use of FRET imaging microscopy to detect protein-protein interactions and protein conformational changes in vivo. Curr. Opin. Struct. Biol. 11: 573-578.

Uetz P, Giot L, Cagney G, Mansfield T A, Judson R S, Knight J R, Lockshon D, Narayan V, Srinivasan M, Pochart P, Qureshi-Emili A, Li Y, Godwin B, Conover D, Kalbfleisch T, Vijayadamodar G, Yang M, Johnston M, Fields S, Rothberg J M. A comprehensive analysis of protein-protein interactions in Saccharomyces cerevisiae. Nature. 2000 403:623-7.

Visintin M, Tse E, Axelson H, Rabbitts T H, Cattaneo A. Selection of antibodies for intracellular function using a two-hybrid in vivo system. Proc Natl Acad Sci USA. 1999. 96:11723-11728.

Volkel T, Korn T, Bach M, Muller R, Kontermann R E. Optimized linker sequences for the expression of monomeric and dimeric bispecific single-chain diabodies. Protein Eng. 2001. 14:815-823.

Walhout A J, Vidal M. High-throughput yeast two-hybrid assays for large-scale protein interaction mapping. Methods. 2001. 24:297-306.

Watson, M A., R. Buckholz and M. P. Weiner. (1996) Vectors encoding alternative antibiotic resistance for use in the yeast two-hybrid system. BioTechniques 21:255-259.

Watters, J. M., Telleman, P. and Junghans, R. P. 1997. An optimised method for cell based phage display panning. Immunotechnology 3: 21-29.

Weiner M P, Costa G L, Schoettlin W, Cline J, Mathur E, Bauer J C. Site-directed mutagenesis of double-stranded DNA by the polymerase chain reaction. Gene. 1994. 151:119-123.

Weiner, M and H. Scheraga. A set of Macintosh computer programs for the design and analysis of synthetic genes. CABIOS (now renamed BioInformatics) 1989. 5:191-198.

Weisman M H, Moreland L W, Furst D E, Weinblatt M E, Keystone E C, Paulus H E, Teoh L S, Velagapudi R B, Noertersheuser P A, Granneman G R, Fischkoff S A, Chartash E K. Efficacy, pharmacokinetic, and safety assessment of adalimumab, a fully human anti-tumor necrosis factor-alpha monoclonal antibody, in adults with rheumatoid arthritis receiving concomitant methotrexate: a pilot study. Clin Ther. 2003. 25:1700-1721.

Whaley, S. R., English, D. S., Hu, E. L., Barbara, P. F. and Belcher, A. M. 2000. Selection of peptides with semiconductor binding specificity for directed nanocrystal assembly. Nature 405: 665-668.

Willats, W. G. T., Gilmartin, P. M., Mikkelsen, J. D. and Knox, J. P. 1999. Cell wall antibodies without immunisation: generation of and use of de-esterified homogalacturonan block-specific antibodies from a naïve phage display library. Plant J. 18: 57-65.

Willats, W. G. T., Rasmussen, S. E., Kristensen, T., Mikkelsen, J. D. and Knox, J. P. 2002a. Sugar-coated microarrays: a novel slide surface for the high throughput analysis of glycans. Proteomics 2(12): in press.

Willats, W. G. T., Steele-King, C. G. and Knox, J. P. 2002b. Antibody techniques. In: P. Gilmartin and C. Bowler (eds.), Molecular Plant Biology: a practical approach, Oxford University Press, Oxford, U. K. Willats, W. G. T., Steele-King, C. G., McCartney, L., Orfila, C., Marcus, S. E. and Knox, J. P. 2000. Making and using antibody probes to study plant cell walls. Plant Physiol. Biochem. 38(1-2): 27-36.

Williams B R, Sompuram S R, Sharon J. Generation of anti-colorectal cancer Fab phage display libraries with a high percentage of diverse antigen-reactive clones. Generation of anti-colorectal cancer fab phage display libraries with a high percentage of diverse antigen-reactive clones. Comb Chem High Throughput Screen. 2002. 5:489-499.

Williams, M. N., Freshour, G., Darvill, A. G., Albersheim, P. and Hahn, M. G. 1996. An antibody Fab selected from a recombinant phage display library detects deesterified pectic polysaccharide rhamnogalacturonan II in plant cells. Plant Cell 8: 673-685.

Wilson, D. S. and Nock, S. 2001. Functional protein microarrays. Curr. Opin. Chem. Biol. 6: 81-85.

Winter, G. 1998a. Synthetic human antibodies and a strategy for protein engineering. FEBS Lett. 430: 92-94.

Winter, G. 1998b. Making antibody and peptide ligands by repertoire selection technologies. J. Mol. Recogn. 11: 126-127.

Winter, G., Griffiths, A. D., Hawkins, R. E. and Hoogenboom, H. R. 1994. Making antibodies by phage display technology. Annu. Rev. Immunol. 12: 433-455. Uetz, P. 2001. Two-hybrid arrays. Curr. Opin. Chem. Biol. 6: 57-62.

Yanisch-Perron C, Vieira J, Messing J. Improved M13 phage cloning vectors and host strains: nucleotide sequences of the M13mp18 and pUC19 vectors. Gene. 1985. 33:103-119.

Zheng L, Baumann U, Reymond J L. Alcala. Production of a functional catalytic antibody ScFv-NusA fusion protein in bacterial cytoplasm. J Biochem (Tokyo). 2003. 133:577-581.

Zucconi, A., Dente, L., Santonico, E., Castagnoli, L. and Cesareni, G.2001. Selection of ligands by panning of domain libraries displayed on phage lambda reveals new potential partners of synaptojanin 1. J. Mol. Biol. 307: 1329-1339.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for making a double-stranded nucleic acid encoding a single-chain antibody, the method comprising:
a) providing a plurality of oligonucleotides, wherein said plurality of oligonucleotides includes:
at least one oligonucleotide encoding a 5′ Framework I sequence, a first germline-encoding CDR1 amino acid sequence, and a 3′ Framework 2 sequence;
at least one oligonucleotide encoding a 5′ Framework 2 sequence, a first germline-encoding CDR2 amino acid sequence, and a 3′ Framework 3 sequence, wherein a region of the 3′ Framework 2 sequence is complementary to a region of the 5′ Framework 2 sequence;
at least one oligonucleotide encoding a 5′ Framework 3 sequence, a first germline-encoding CDR3 amino acid sequence, and a 3′ Framework 4 sequence, wherein a region of the 3′ Framework 3 sequence is complementary to a region of the 5′ Framework 3 sequence;
at least one oligonucleotide encoding a 5′ Framework 4 sequence, a linker sequence, and a 3′ Framework 5 sequence, wherein a region of the 3′ Framework 4 sequence is complementary to a region of the 5′ Framework 4 sequence at least one oligonucleotide encoding a 5′ Framework 5 sequence, a first germline-encoding CDR4 amino acid sequence, and a 3′ Framework 6 sequence, wherein a region of the 3′ Framework 5 sequence is complementary to a region of the 5′ Framework 5 sequence;
at least one oligonucleotide encoding a 5′ Framework 6 sequence, a first germline-encoding CDR5 amino acid sequence, and a 3′ Framework 7 sequence, wherein a region of the 3′ Framework 6 sequence is complementary to a region of the 5′ Framework 6 sequence; and
at least one oligonucleotide encoding a 5′ Framework 7 sequence, a first germline-encoding CDR6 amino acid sequence, and a 3′ Framework 8 sequence, wherein a region of the 3′ Framework 8 sequence is complementary to a region of the 5′ Framework 7sequence;
b) annealing said oligonucleotides, thereby yielding a plurality of primed oligonucleotide complexes;
c) combining the primed oligonucleotide complexes with a polymerase, one or more nucleotides, and optionally a ligase,
thereby generating double-stranded nucleic acid sequence encoding a single-chain antibody.

2. The method of claim 1, where said a plurality of oligonucleotides further comprises an oligonucleotide encoding a 5′ Framework I sequence, a second germline-encoding CDR1 amino acid sequence, and a 3′ Framework 2 sequence, wherein said second germline-encoding CDR1 amino acid sequence is different than the first germline-encoding CDR1 amino acid sequence

3. The method of claim 1, where said a plurality of oligonucleotides further comprises further comprising an oligonucleotide encoding a 5′ Framework 1 sequence, a third germline-encoding CDR1 amino acid sequence, and a 3′ Framework 2 sequence, wherein said third germline-encoding CDR1 amino acid sequence is different than the first germline-encoding CDR1 amino acid sequence and the second germline-encoding CDR1 amino acid sequence.

4. The method of claim 1, where said a plurality of oligonucleotides further comprises further comprising an oligonucleotide encoding a 5′ Framework 2 sequence, a second germline-encoding CDR2 amino acid sequence, and a 3′ Framework 3 sequence, wherein a region of the 3′ Framework 2 sequence is complementary to a region of the 5′ Framework 2 sequence, and wherein said second germline-encoding CDR2 amino acid sequence is different than the first germline-encoding CDR2 amino acid sequence

5. The method of claim 1, wherein said at least one oligonucleotide encoding a 5′ Framework 1 sequence, a germline-encoding CDR1 amino acid sequence, and a 3′ Framework 2 sequence comprises 5′ to the 5′ Framework 1 sequence a first priming sequence.

6. The method of claim 1, wherein said at least one oligonucleotide encoding a 5′ Framework 8 sequence, a germline-encoding CDR6 amino acid sequence, and a 3′ Framework 8 sequence comprises 3′ to the 3′ Framework 8 sequence a second priming sequence.

7. The method of claim 1, futher comprising oligonucleotides complemenatry to the plurality of oligonucleotides.

8. The method of claim 1, further comprising the step of isolating said nucleic acid sequence encoding a single-chain antibody.

9. The method of claim 8, wherein said isolating step is by size exclusion.

10. A nucleic acid encoding a single-chain antibody obtained by the method according to claim 1.

11. A single-chain antibody obtained by the method according to claim 1.

12. A polynucleotide library comprising nucleic acid sequences obtained by the method according to claim 1.

13. The method of claim 1, further comprising the step of expressing the resulting protein encoded by the nucleic acid sequence and screening said protein for a desired property.

14. The method of claim 13, further comprising the step of partitioning the nucleic acids encoding said protein of desired property and amplifying the nucleic acids to yield an enriched mixture of nucleic acids encoding a protein with a desired property.

15. The method of claim 13, wherein said desired property is antigen binding.

16. The method of claim 14, wherein said enriched mixture of nucleic acids encode a protein which binds to an antigen at high affinity.

17. The method of claim 16, wherein said high affinity is a Kd of at least 10-6

18. The method of claim 13, wherein said screening is by a yeast two-hybrid system.

19. A vector comprising the nucleic acid of claim 1.

20. A host cell comprising the vector of claim 18.

21. A kit comprising the vector of claim 19.

22. The host cell of claim 20, wherein said cell is a bacterial cell, a yeast cell or a mammalian cell.

23. A method of producing a single chain antibody comprising culturing the host cell of claim 20 so that the polypeptide is produced.

24. A kit comprising nucleic acid of claim 1.

25. An expression vector comprising:
i) an activation domain polynucleotide sequence in functional combination with a yeast promoter sequence,
ii) a polynucleotide sequence encoding a bacteriophage coat protein or fragment thereof in functional combination with a bacterial promoter sequence,
iii) a protein fusion site, and
iv) a suppressible stop codon.

26. The expression vector of claim 25, wherein said vector is capable of expressing a polypeptide sequence in two or more species when contained in a cell thereof.

27. The expression vector of claim 25, wherein said species are selected from the group consisting of a yeast, a bacterium, and a mammal.

28. The expression vector of claim 25, wherein said activation domain is selected from the group consisting of a B42 activation domain, a VP16 activation domain, a GAL4 activation domain, and an NF-κB activation domain.

29. The expression vector of claim 25, further comprising a nuclear localization sequence.

30. The expression vector of claim 29, wherein said nuclear localization sequence is selected from the group consisting of an SV40 large T antigen nuclear localization sequence, a Matcc2 nuclear localization sequence, a nucleoplasmin nuclear localization sequence, and a c-myc nuclear localization sequence.

31. The expression vector of claim 25, further comprising an epitope tag sequence.

32. The expression vector of claim 31, wherein said epitope tag is selected from the group consisting of a V5 epitope tag, a 6-His epitope tag, a c-myc tag, a Flag tag, a GFP tag, a GST tag, a HA tag, a luciferase tag, a Protein C tag, an S-tag, a T7 tag, a thioredoxin tag, and a VSV-g tag.

33. The expression vector of claim 25, further comprising a yeast transcription termination signal.

34. The expression vector of claim 33, wherein said yeast transcription termination signal is a CYC1 transcription termination signal.

35. The expression vector of claim 25, further comprising an origin of replication.

36. The expression vector of claim 35, wherein said origin of replication is a 2μ origin.

37. The expression vector of claim 25, further comprising a mammalian promoter sequence.

38. The expression vector of claim 37, wherein said mammalian promoter sequence is selected from the group consisting of CMV immediate early, HSV thymidine kinase, early and late SV40, a retroviral LTR, elongation factor-1a (EF-1a), and mouse metallothionein-I.

39. The expression vector of claim 25, wherein said bacteriophage coat protein or fragment thereof is selected from the group consisting of gpIII, gpVII, and gpVIII.

40. The expression vector of claim 25, wherein said bacterial promoter sequence is selected from the group consisting of lacl, lacZ, T3, T7, gpt, lambda P_{R}, P_{L} and TRP.

41. The expression vector of claim 25, wherein said yeast activation domain is located downstream of said yeast promoter sequence, said bacterial promoter sequence is in-frame with and downstream of said activation domain, said protein fusion site is downstream of said bacterial promoter sequence, said suppressible stop codon is downstream of said protein fusion site, and said polynucleotide sequence encoding said bacteriophage coat protein is downstream of said suppressible stop codon.

42. The expression vector of claim 25, further comprising a yeast selection marker.

43. The expression vector of claim 42, wherein said yeast selection marker is selected from the group consisting of URA3, HIS3, LEU2, TRP1 and LYS2.

44. The expression vector of claim 25, further comprising a bacterial selection marker.

45. The expression vector of claim 44, wherein said bacterial selection marker is selected from the group consisting of ampicillin, streptomycin, gentamicin, ofloxacin, tetracycline, kanamycin, spectinomycin, and chloramphenicol.

46. The expression vector of claim 25, further comprising the nucleic acid of claim 1.

47. The expression vector of claim 25, wherein said vector is a plasmid vector, a BAC vector, a cosmid vector, or a YAC vector.

48. A method for making a polynucleotide library of double-stranded nucleic acida encoding single-chain antibodies, the method comprising:
a) providing a plurality of oligonucleotides, wherein said plurality of oligonucleotides includes:
at least one oligonucleotide encoding a 5′ Framework 1 sequence, a first germline-encoding CDR1 amino acid sequence, and a 3′ Framework 2 sequence;
at least one oligonucleotide encoding a 5′ Framework 2 sequence, a first germline-encoding CDR2 amino acid sequence, and a 3′ Framework 3 sequence, wherein a region of the 3′ Framework 2 sequence is complementary to a region of the 5′ Framework 2 sequence;
at least one oligonucleotide encoding a 5′ Framework 3 sequence, a first germline-encoding CDR3 amino acid sequence, and a 3′ Framework 4 sequence, wherein a region of the 3′ Framework 3 sequence is complementary to a region of the 5′ Framework 3 sequence;
at least one oligonucleotide encoding a 5′ Framework 4 sequence, a linker sequence, and a 3′ Framework 5 sequence, wherein a region of the 3′ Framework 4 sequence is complementary to a region of the 5′ Framework 4 sequence at least one oligonucleotide encoding a 5′ Framework 5 sequence, a first germline-encoding CDR4 amino acid sequence, and a 3′ Framework 6 sequence, wherein a region of the 3′ Framework 5 sequence is complementary to a region of the 5′ Framework 5 sequence;
at least one oligonucleotide encoding a 5′ Framework 6 sequence, a first germline-encoding CDR5 amino acid sequence, and a 3′ Framework 7 sequence, wherein a region of the 3′ Framework 6 sequence is complementary to a region of the 5′ Framework 6 sequence; and
at least one oligonucleotide encoding a 5′ Framework 7 sequence, a first germline-encoding CDR6 amino acid sequence, and a 3′ Framework 8 sequence, wherein a region of the 3′ Framework 8 sequence is complementary to a region of the 5′ Framework 7sequence;
b) annealing said oligonucleotides, thereby yielding a plurality of primed oligonucleotide complexes;
c) combining the primed oligonucleotide complexes with a polymerase, one or more nucleotides, and optionally a ligase;
d) generating double-stranded nucleic acid sequence encoding a single-chain antibody and
e) inserting said double-stranded nucleic acid sequence into a vector.

49. The method according to claim 48, futher comprising the step of screening the library for a protein with a desired property.
